(19) United States
(12) Patent Application Publication
(10) Pub. No.: US 2019/0030128 A1
(43) Pub. Date: Jan. 31, 2019

(54) Compositions and Methods for Treatment of Central Nervous System Diseases

(71) Applicant: Medgenics Medical Israel Ltd., Misgav (IL)

(72) Inventors: Garry NEIL, New Hope, PA (US); Nir SHAPIR, Misgav (IL); Reem MIARI, Sakhnin (IL)

(73) Assignee: Medgenics Medical Israel Ltd., Misgav (IL)

(21) Appl. No.: 16/069,355

(22) PCT Filed: Jan. 10, 2017
(86) PCT No.: PCT/IB2017/000076

§ 371 Date: Jul. 11, 2018

Related U.S. Application Data
(60) Provisional application No. 62/277,365, filed on Jan. 11, 2016.

(51) Int. Cl. A61K 38/18; A61K 9/00; A61M 31/00; C07K 16/28

(52) U.S. Cl. CPC A61K 38/1816; A61K 9/0024; A61P 35/00; C07K 16/2878; A61K 9/0085; A61M 31/002

(57) ABSTRACT

Micro-organ compositions and methods of implanting into the central nervous system (CNS) for the treatment of CNS-related diseases are encompassed. Specifically, the disclosure provides methods for treating disorders including cancer and lysosomal storage diseases, the methods comprising implanting a micro-organ into the CNS, wherein the micro-organ secretes a recombinant protein, and wherein the micro-organ is maintained in the CNS, and secretes protein, for at least seven days.

### FIELD

The invention relates to Transduced Autologous Restorative Gene Therapy (TARGT™) for sustained delivery of proteins to the central nervous system.

### BACKGROUND

Delivery of therapeutic proteins, including antibodies, over an extended period of time is advantageous for treating a number of diseases that affect the central nervous system (CNS), which includes the brain and the spinal cord. However, the blood brain barrier controls the passage of substances from the blood to the CNS and impedes the delivery of therapeutic macromolecules to the brain and spinal cord.

A number of strategies have been investigated to allow delivery of therapeutic proteins to the brain (see Calias P et al., Pharmacology & Therapeutics 144:114-122 (2014)). Delivery options that allow or facilitate delivery of proteins across the blood brain barrier have been investigated, such as liposomes, prodrugs, chimeric peptides, and proton-coupled oligopeptide transporters. However, these facilitated delivery techniques have met with limited success. Direct injection of therapeutic proteins by intrathecal (IT) or intracerebroventricular (ICV) delivery, thereby bypassing the blood brain barrier, has also been studied. While IT and ICV administration of agents has shown success in exerting local effects of therapeutics, such as for pain management, treatment of spasticity, and localized chemotherapy, direct central administration of protein therapeutics has yet to show a large degree of penetration into the CNS beyond the site of injection, thus limiting its utility. New delivery methods for obtaining widespread delivery of protein therapeutics throughout the CNS are needed.

A number of diseases or conditions could be treated by therapeutic proteins that are able to be delivered to the CNS. For example, therapeutic antibodies have shown efficacy for treatment of cancer, but their efficacy in treatment of primary and metastatic CNS cancer is limited by their low delivery across the blood brain barrier. In addition, a number of genetic disorders, including lysosomal storage diseases, involving the CNS are known to be due to genetic defects that cause a lack of production of specific proteins in the brain. However, treatment of CNS disorders with replacement protein therapies are similarly hampered by poor delivery of protein therapeutics to the CNS, and thus treatments that avoid blood brain barrier concerns are needed.

We have previously described that human dermal micro-organs can deliver therapeutic polypeptides (see US Application 20150118187). Herein, we describe the successful delivery of human therapeutic proteins within the CNS using TARGT. Therapeutic protein was detected beyond the site of implantation, and protein production was sustained for extended periods of time. In some instances, protein produced from TARGT was detected in serum. In vivo production of therapeutic proteins within the CNS is a means to overcome limitations seen with other attempts to deliver therapeutic proteins to the CNS. In addition, the TARGT system of dermal micro-organs have the distinct advantage of allowing reversible therapy, as the MOs can be removed. The present invention thus overcomes multiple disadvantages seen with other means of delivering therapeutic proteins to the CNS.

### SUMMARY

This invention involves the use of centrally implanted micro-organs for production of therapeutic proteins in the CNS. In one embodiment, the invention comprises a method for treating cancer comprising implanting a micro-organ into the central nervous system (CNS), wherein the micro-organ secretes a recombinant protein, and wherein the micro-organ is maintained in the CNS, and secretes protein, for at least seven days.

In some embodiments, the micro-organ is implanted at the same time as a procedure for biopsy, removal, or debulking of a CNS tumor.

In some embodiments, the cancer is a primary CNS tumor(s) or a tumor(s) secondary to a cancer with origins outside of the CNS. In some embodiments, the cancer in the CNS is secondary to colon, kidney, melanoma, lung, ovarian, breast, or testicular cancer. In some embodiments, the cancer is or has an astrocytoma, glioblastoma, glioma, lymphoma, including CNS lymphoma, or medulloblastoma.

In some embodiments, the protein secreted by the micro-organ is an antibody. In some embodiments, the antibody is trastuzumab, anti-PD1, cetuximab, an immune check-point antibody, or rituximab.

In some embodiments, the method for treating cancer further comprises administration of a biologic or non-biologic chemotherapeutic agent.

In another embodiment, the invention comprises a method for treating a lysosomal storage disease comprising implanting a micro-organ into the central nervous system (CNS), wherein the micro-organ secretes a recombinant protein, and wherein the micro-organ is maintained in the CNS, and secretes protein, for at least seven days. In some embodiments, the lysosomal storage disease is Hunter syndrome, Fabry disease, Infantile Batten disease (CNL1), Classic late infantile Batten disease (CNL2), Hurler syndrome, Krabbe disease, Niemann-Pick A, Niemann-Pick B, Pompe disease, Batten disease, Gaucher disease, or Tay Sachs disease. In some embodiments, the recombinant protein replaces a gene product that is not expressed or that is misexpressed due to a genetic mutation.

In some embodiments, secretion of the recombinant protein is measurable in the CNS for a sustained period of time of at least one week, at least one month, at least two months, at least three months, at least four months, at least five months, at least six months, at least seven months, at least eight months, at least nine months, at least ten months, at least eleven months, or at least twelve months. In some embodiments, secretion of the recombinant protein is measurable outside of the CNS for a sustained period of time of at least one week, at least one month, at least two months, at least three months, at least four months, at least five months, at least six months, at least seven months, at least eight months, at least nine months, at least ten months, at least eleven months, or at least twelve months.

In some embodiments, the secretion of the recombinant protein within the CNS is monitored by measurement of levels in the cerebrospinal fluid. In some embodiments, a catheter is implanted to allow periodic measurement of cerebrospinal fluid. In some embodiments, the level of recombinant protein is measured via imaging of the brain and/or spinal cord. In some embodiments, the level of the recombinant protein the CNS determines the timing of removal of the micro-organ(s) and the timing of subsequent implantations of additional micro-organ(s).

In some embodiments, the invention comprises a method of preparing a micro-organ for implantation into the CNS comprising i) removing a micro-organ of non-CNS tissue; ii) maintaining the micro-organ in vitro for 1 to 7 days; iii) transducing the micro-organ with a viral vector comprising a therapeutic protein; and iv) freezing the transduced micro-organ. In some embodiments, steps iv) and iii) are reversed such that the micro-organ is frozen and thawed prior to transduction.

In some embodiments, the invention comprises a method of implanting a micro-organ into the CNS, comprising making an incision in the dura and inserting a micro-organ, wherein the micro-organ secretes a recombinant protein into the sub-dural space and outside of the sub-dural space. In some embodiments, the micro-organ is inserted into the spine, cisterna magna, ventricular system space of the brain, brain convexity, or brain parenchyma.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIG. 1 provides an experimental plan for a study to assess a variety of different pre-implantation procedures. Autologous micro-organs (MOs) were implanted into the cisterna magna of Lewis rats, and samples were assessed four days after implantation.

FIG. 2 shows DAPI (left) and CD68 (right) staining in MO #2-4 at 4 days post implantation in implantation study #2. The MOs were frozen and then thawed in fetal bovine serum (FBS) with no rinsing prior to implantation. Large numbers of cells were observed around the periphery and within the MO. Many of these cells were confirmed to be CD68⁺ based on immunohistochemistry.

FIG. 3 shows CD68 staining in representative MOs following implantation into the cisterna magna of Lewis rats. MO #3-4 were frozen, thawed in rat serum, and washed six times with PBS prior to implantation in implantation study #3. Explantation was done at 4 days post implantation followed by staining. No CD68⁺ cells were observed at the periphery or within the MO. However, some artifactual staining was found on the edges where the MO lifted.

FIG. 4 shows CD68 staining in representative MOs following implantation into the cisterna magna of Lewis rats. MO #3-9 was frozen, thawed in fetal bovine serum (FBS), and washed six times with PBS prior to implantation in implantation study #3. Explantation was done at 4 days post implantation followed by staining. CD68⁺ cells were observed at the periphery and partially within the implanted MO.

FIG. 5 shows an experimental plan for a study, wherein MOs implanted in the cisterna magna of Lewis rats were assessed at 4, 7, or 14 days post-implantation.

FIGS. 6A-C show H&E staining of MO #4-1 at 4 days post-implantation in implantation study #4. This MO was significantly larger than MOs used in previous studies; thus, the surgically-created defect in the cisterna magna was enlarged prior to MO insertion. The additional trauma resulted in greater cellular infiltration on the MO periphery and few cells observed mid-MO. The MO section contracted and wrinkled during staining. Scale bars 4×=500 μm and 10×=200 μm.

FIGS. 7A-D show H&E staining of MO #4-2 at 7 days post-implantation (7A-7C) and DAPI staining to measure live cells (7D).

FIGS. 8A-C show CD68 staining of MO #4-2 at 7 days post-implantation in implantation study #4. CD68⁺ cells were observed on the MO periphery but not within the MO. Scale bars 4×=500 μm, 10×=200 μm, and 20×=100 μm.

FIGS. 9A-C show H&E staining of MO #4-3 at 14 days post-implantation in implantation study #4. Uniform numbers of cells were observed throughout the MO with few cells on the MO periphery. Scale bars 4×=500 μm and 10×=200 μm.

FIGS. 10A-C show CD68 staining of MO #4-3 at 14 days post-implantation in implantation study #4. CD68⁺ cells (macrophages and activated microglia) were observed on the MO periphery but not within the MO. Scale bars 4×=500 μm, 10×=200 μm, and 20×=100 μm.

FIGS. 11A-C show ionized calcium-binding adapter molecule 1 (IBA-1) staining of MO #4-3 at 14 days post-implantation in implantation study #4. IBA-1⁺ cells (microglia) were observed on the MO periphery but not within the MO. Scale bars 4×=500 μm, 10×=200 μm, and 20×=100 μm.

FIG. 12 shows an experimental plan for an implantation study, wherein TARGT_{EPOs} (see, e.g., U.S. Pat. No. 9,155,749) were implanted in the cisterna magna of Lewis rats and assessed at 4 days post-implantation.

FIG. 13 shows in vitro secretion of human erythropoietin (hEPO) by rat TARGT_{EPOs}.

FIGS. 14A-B show H&E staining of TARGT_{EPO} #5-4 at 4 days post-implantation in implantation study #5. The TARGT pulled out of the brain upon explantation. Although the cellular infiltrate surrounding the TARGT may have detached when the TARGT was removed from the brain, little cellular infiltration was observed into the TARGT. Scale bars A) 500 μm and B) 200 μm.

FIGS. 15A-D show H&E staining (A and C) and CD68 staining (B and D) of TARGT_{EPO} #5-5 at 4 days post-implantation in implantation study #5. The TARGT remained in the brain upon explantation. Based on H&E staining, uniform numbers of cells were observed throughout the TARGT without significant cellular infiltration from the periphery. CD68⁺ cells (macrophages and activated microglia) were observed on the TARGT periphery but not within the TARGT. Scale bars A) and B) 500 μm and C) and D) 200 μm.

FIGS. 16A-C show higher magnification H&E staining and CD68 staining of TARGT_{EPO} #5-5 at 4 days post-implantation in implantation study #5. Based on H&E staining, uniform numbers of cells were observed throughout the TARGT without significant cellular infiltration from the periphery. CD68⁺ cells (macrophages and activated microglia) were observed on the TARGT periphery; an occasional CD68⁺ cell may have been located within the TARGT (arrow in B). Scale bars A) and B) 100 μm and C) 50 μm.

FIGS. 17A-C show the in vitro secretion profile of adalimumab from pig TARGT-adalimumabs. FIG. 17A shows concentration of adalimumab per TARGT per day up to 42 days after harvesting. FIGS. 17B (reducing conditions) and 17C (non-reducing conditions) show western blot analysis of adalimumab secreted from 2 separate pig TARGT-adalimumabs (TARGT-1 and TARGT-2) in comparison to commercial adalimumab (Humira®, labeled as “Std.”).

FIG. 18 shows in vitro secretion profile of pig TARGT-adalimumabs maintained in 100% pig CSF compared to those maintained in DMEM-F12 media supplemented with 10% serum.

FIG. 19A-C show in-vivo results of pig TARGT-adalimumabs implanted in the cisterna magna. FIG. 19A shows adalimumab levels measured in CSF sampled from cisterna magna (CM), lumbar (LP), sub-dura (head) and pig serum 7 days post-implantation of TARGT-adalimumabs into pig cisterna magna. FIGS. 19B-19C shows H&E staining on pig TARGT-adalimumabs excised from pig cisterna magna one week post implantation. H&E stained images were obtained at 4× (19B) and 10× (19C) magnification

### DESCRIPTION OF THE EMBODIMENTS

### Definitions

“Treatment” as used herein, covers any administration or application of a therapeutic for disease in a mammal, including a human, and includes inhibiting the disease or progression of the disease, inhibiting or slowing the disease or its progression, arresting its development, partially or fully relieving the disease, preventing the onset of the disease, or preventing a recurrence of symptoms of the disease.

“Centrally” implanted or administered as used herein, means implanted or administered into the central nervous system (CNS). “Peripherally” implanted or administered means implanted outside of the CNS.

As used herein “micro-organ,” “microorgan,” and “MO,” are used interchangeably throughout to refer to an explant of mammalian tissue that is retrieved from a donor and then maintained ex vivo for future transplantation. The donor may be the same individual into whom the micro-organ is later implanted. The micro-organ may be generated from dermal tissue, in which case it is referred to as a “dermal micro-organ,” or “DMO”. In some cases, this dermal micro-organ is generated from a tummy tuck procedure.

As used herein, “TARGT” refers to micro-organs that have been transduced with a virus containing an expression construct using the TARGT (Transduced Autologous Restorative Gene Therapy) technology. In short, the TARGT procedure involves harvesting a micro-organ, culturing the micro-organ in vitro, and ex vivo transduction of the micro-organ with a viral vector comprising a nucleic acid encoding a protein. The secretion of protein from the micro-organ may be quantitated and verified, and the transduced micro-organ subsequently implanted into subject or patient. When a TARGT is used to generate a protein, it is termed “TARGT-protein,” where “protein” is replaced with the name of the relevant protein. In one embodiment of the present invention, a nucleic acid encoding a heavy chain and light chain of an antibody is provided within a viral vector cassette, wherein the heavy and light chain are separated by a site cleavable after translation, such that the TARGT-antibody fulfills all the expression, folding, and secretion requirements to generate active antibody both in vitro and in vivo.

As used herein, “TARGT_{CNS}” is synonymous with “TARGT-CNS”, and refers to any protein-producing micro-organ that is implanted in the central nervous system.

As used herein, “protein” refers to a molecule consisting of amino acids. The protein may be composed of natural or non-natural amino acids. The term protein may be used interchangeably with polypeptide. A protein may be a sequence of amino acids encoded by a genome of an organism or may be a sequence of amino acids that is entirely artificial and not represented in any genome. A protein may refer to a construct that corresponds to the full-length of a gene product that is encoded by a genome. Protein is also inclusive of a peptide that does not contain the full amino acid sequence of a full-length gene product. A protein may also correspond to a sequence that has been changed or optimized compared to the wild-type sequence encoded by a genome. Accordingly, all proteins, peptides, antibodies and antibody fragments are proteins according to the invention.

“Construct” and “cassette” are used interchangeably throughout this application.

As used herein, “antibody” refers to full length as well as functional fragments or variants thereof, so long as the functional fragment or variant is capable of binding antigen or epitope. For example, the term “antibody” refers to antibodies portions, fragments, regions, peptides, single chains, bispecific antibodies and derivatives thereof so long as they bind to antigen or epitope.

As used herein the term “combination” is used in its broadest sense and means that a subject is treated with at least two therapeutic regimens. Treatment can be at the same time (e.g. simultaneously or concomitantly), or at different times (e.g. consecutively or sequentially), or a combination thereof. For the purposes of the present disclosure, administering at the same time (e.g., simultaneously) refers to administering the TARGT-protein and other therapeutic, such as, for example, a chemotherapeutic agent, together via same TARGT-protein or in separate delivery devices. As used herein administering at different times (e.g., sequentially) refers to administering the TARGT-protein of the combination therapy a few hours to days, weeks and even months apart from the other therapeutic.

A. Micro-Organs Producing Proteins in the CNS

We herein show successful production of recombinant protein from dermal micro-organs implanted within the CNS. Utilizing rat and porcine models, we show that when implanted into the CNS, dermal micro-organs deliver therapeutically relevant levels of protein throughout the cerebrospinal fluid (CSF). The centrally implanted micro-organ does not sustain substantial damage by the host environment, and is capable of secreting protein for extended periods of time.

1. Micro-Organs

The generation and use of a dermal micro-organ for expression of proteins has been previously described (see US Application 20150118187). However, implantation of micro-organs into the CNS has not been previously shown. The CNS was believed to be an inappropriate implantation site for at least the reason that micro-organ rejection and ineffectiveness were predicted. For example, it was expected that the CNS would not support survival of a micro-organ long enough for the micro-organ to integrate, as the dermal tissue structure and content is different from brain tissue and may lead to rejection of the micro-organ. Additionally, one might expect that implantation of a micro-organ could exert pressure on the CNS tissue due to the space restrictions of the skull and vertebrae, leading to changes in the behavior of the micro-organ as well as the host response.

In one embodiment, the micro-organ is dermal micro-organ. In some embodiments, the micro-organ is a genetically modified dermal micro-organ. Dermal micro-organs may comprise a plurality of dermis components, wherein in one embodiment dermis is the portion of the skin located below the epidermis. These components may comprise fibroblast cells, epithelial cells, other cell types, bases of hair follicles, nerve endings, sweat and sebaceous glands, and blood and lymph vessels. In one embodiment, a dermal micro-organ may comprise some fat tissue, wherein in another embodiment, a dermal micro-organ may not comprise fat tissue. In some embodiments, the dermal micro-organ is generated from tissue collected from a tummy tuck procedure. In one embodiment the dermal micro-organ does not comprise epidermis. In some embodiments, the dermal micro-organ comprises epidermis.

In some embodiments, a therapeutic protein is produced by the micro-organ. In some embodiments, the micro-organ is used to generate a TARGT that expresses a therapeutic protein (i.e., TARGT-protein). In some embodiments, the TARGT-protein is a dermal micro-organ lacking epidermis.

In some embodiments, the protein produced by the micro-organ are antibodies. In some embodiments, the micro-organ is used to generate a TARGT that expresses antibody (i.e., TARGT-antibody). In some embodiments the TARGT-antibody is a dermal micro-organ lacking epidermis.

In some embodiments, the micro-organ is autologous, meaning it is derived from tissue harvested from the same subject in which it is implanted after transduction. In some embodiments, the donor may be a rodent, such as a mouse or rat, of an in-bred strain, wherein the recipient of the micro-organ after transduction using the TARGT system is a rodent of the same in-bred strain. In some embodiments, the donor may be human. In some embodiments, the micro-organ is not autologous, meaning the micro-organ is derived from tissue harvested from one or more subjects and implanted into one or more subjects, wherein the subjects are not the same as the subjects from which the tissue was harvested.

2. Viral Vectors Transduced

Any methodology known in the art can be used for genetically altering the micro-organ explant to allow expression of the therapeutic protein. Any one of a number of different vectors can be used in embodiments of this invention, such as viral vectors, plasmid vectors, linear DNA, etc., as known in the art, to introduce an exogenous nucleic acid fragment encoding a therapeutic agent into target cells and/or tissue. In some embodiments, viral vectors may be used to transduce the micro-organ, such as adenovirus vectors, helper-dependent adenovirus vectors (HDAd), adeno-associated virus vectors, and retroviral vectors (such as lentivirus vectors). In some embodiments, the viral vector is an HDAd that has been modified, such as being a gutless, gutted, mini, fully deleted, high-capacity, 4, or pseudo adenovirus. In some embodiments, the HDAd has been deleted of all viral coding sequences, expresses no viral proteins, or is a non-replicating vector.

3. Expression Constructs

In one embodiment, expression constructs containing full-length or partial-length therapeutic protein were cloned into the multiple cloning site of an HDAd viral vector MAR-EF1a construct containing regulatory elements (see US Application 20150118187). In some embodiments, the full-length or partial-length therapeutic proteins comprise a wild-type human sequence for the protein. In some embodiments, the sequence of the full-length or partial-length therapeutic protein comprises a modified or optimized sequence for the protein.

In some embodiments, the therapeutic protein is EPO (SEQ ID No:19). In some embodiments, the sequence of the therapeutic protein is an optimized sequence of EPO (SEQ ID No:20). In some embodiments, the virus used to transduce the micro-organ is HDΔ28E4-MAR-EF1a-optHumanEPO-1 (SEQ ID No:18).

In some embodiments, the therapeutic protein is an enzyme. In some embodiments, the therapeutic protein is an enzyme that is not expressed or misexpressed in a genetic disorder. In some embodiments, the therapeutic protein is idursulfase, agalsidase alfa, agalsidase beta, palmitoyl-protein thioesterase, tripeptidyl peptidase, alpha-L-iduronidase, galactocerebrosidase, acid sphingomyelinase, NPC-1, or acid alpha-glucosidase. In some embodiments, the therapeutic protein is not an enzyme.

In some embodiments, the therapeutic protein is an antibody. In some embodiments, the therapeutic protein is an antibody that has been engineered. In some embodiments, the therapeutic protein is adalimumab. In some embodiments, the therapeutic protein is trastuzumab, anti-PD1, cetuximab, an immune check-point antibody, or rituximab. In some embodiments, the antibody binds to or interacts with TNF-alpha, human epidermal growth factor receptor 2 (HER2), or CD20. The invention is not limited by any specific antibody expressed by the TARGT or by the site of action of this antibody expressed by the TARGT. In some embodiments, the therapeutic protein is not an antibody.

In some embodiments, the virus used to transduce the micro-organ contains a construct with the light chain and heavy chain of adalimumab. In some embodiments, the light chain and heavy chain of adalimumab are optimized. In some embodiments, the virus used to transduce the micro-organ is pAd-MAR-EF1a-opt hTNF1 (SEQ ID No:16). In some embodiments, the virus used to transduce the micro-organ is pAd-MAR-EF1a-opt hTNF3 (SEQ ID No:17). In some embodiments, the virus used to transduce the micro-organ contains a TNF1 construct comprising the nucleic acids of SEQ ID No:14, or nucleic acids having at least 95%, 90%, 85%, or 80% homology to SEQ ID No: 14. In some embodiments, the virus used to transduce the micro-organ comprises the nucleic acids of SEQ ID No:15, or nucleic acids having at least 95%, 90%, 85%, or 80% homology to SEQ ID No: 15. In some embodiments, the micro-organ is transduced with a virus comprising the nucleic acids of SEQ ID No: 1, or nucleic acids having at least 95%, 90%, 85%, or 80% homology to SEQ ID No: 1. In some embodiments, the micro-organ is transduced with a virus comprising the nucleic acids of SEQ ID No: 2, or nucleic acids having at least 95%, 90%, 85%, or 80% homology to SEQ ID No: 2 In some embodiments, the micro-organ is transduced with a virus comprising the nucleic acids of SEQ ID No: 3, or nucleic acids having at least 95%, 90%, 85%, or 80% homology to SEQ ID No: 3. In some embodiments, the micro-organ is transduced with a virus comprising the nucleic acids of SEQ ID No: 4, or nucleic acids having at least 95%, 90%, 85%, or 80% homology to SEQ ID No: 4. In some embodiments, the micro-organ is transduced with a virus comprising the nucleic acids of SEQ ID Nos: 1 or 2 (one of the light chains), or nucleic acids having at least 95%, 90%, 85%, or 80% homology to SEQ ID Nos: 1 or 2 in combination with SEQ ID No: 3 or 4 (one of the heavy chains), or nucleic acids having at least 95%, 90%, 85%, or 80% homology to SEQ ID No: 3 or 4.

In another embodiment, expression constructs containing partial length light and heavy chains of antibodies with signaling sequences and a separation site cleavable after translation are cloned into the multiple cloning site of an HDAd viral vector MAR-EF1a construct containing regulatory elements (see US Application 20150118187). The separation site allows stoichiometric expression of both the light chain and heavy chain of the antibody from a single cassette. In some embodiments, the components of the expression construct are regulatory elements, separation sites (to allow stoichiometric expression), antibody elements, signal sequences, and/or a polyadenylation site.

In some embodiments, the therapeutic protein expressed by the TARGT is selected based on the association of an enzyme with a lysosomal storage disease. In other embodiments, the therapeutic protein expressed by the TARGT is selected based on known efficacy of an antibody for therapeutic purposes. As such, the following is a non-inclusive list of therapeutic proteins that may be predicted to have efficacy in treating a disease of the CNS.

| Indication | Protein |
|---|---|
| Brain metastasis of breast cancer | Herceptin (Ab) |
| Glioblastoma (primary brain | Anti PD-1/Cetuximab/immune check |
| tumor) | point antibody (Abs) |
| CNS Lymphoma | Rituximab (Ab) |
| CNS metastasis of melanoma | Anti PD-1 (Ab) |
| Hunter syndrome | Idursulfase (enzyme) |
| Fabry disease | Agalsidase alfa (enzyme) |
| Infantile Batten disease (CNL1) | Palmitoyl-protein thioesterase (enzyme) |
| Classic late infantile Batten | Tripeptidyl Peptidase (enzyme) |
| disease (CNL2) | |
| Hurler syndrome | Alpha-L-iduronidase (enzyme) |
| Krabbe disease | Galactocerebrosidase (enzyme) |
| Niemann-Pick A | Acid sphingomyelinase (enzyme) |
| Niemann-Pick C | NPC-1 (enzyme) |
| Pompe | Acid alpha-glucosidase (enzyme) |

4. Regulatory Elements

In some embodiments, the vector comprises a nucleic acid sequence encoding an antibody operably linked to an upstream MAR regulatory sequence. In some embodiments, at least one additional regulatory sequence to the MAR regulatory sequence is also present.

In some embodiments, the additional regulatory sequences may comprise a MAR sequence (or two MAR sequences), a CAG promoter sequence, an EF1-alpha promoter sequence, and/or a woodchuck hepatitis virus post-transcriptional regulation element (WPRE sequence). In certain embodiments, the sequence of the EF1-alpha promoter corresponds to SEQ ID NO: 7. In certain embodiments, the CpG free MAR from human beta globin gene (SEQ ID NO: 8) may be one or more of the MAR sequences. In certain embodiments, the MAR 5′ region from human IFN-beta gene (SEQ ID NO: 9) may be one or more of the MAR sequences. In certain embodiments, the CMV enhancer (SEQ ID NO: 6) may be used as a regulatory sequence.

As regulatory sequences are well-known to those skilled in the art, the present invention is not limited by a specific regulatory sequences. Those skilled in the art would understand that regulatory sequences may be tested and selected based upon the optimal level of expression of the resulting therapeutic protein. Any regulatory sequence or set or regulatory sequences that allow expression of antibodies encoded by the sequences of the cassette would be appropriate, based upon the desired level of protein expression for a particular micro-organ.

5. Separation Sites

Those skilled in the art of generation of recombinant antibodies would understand that stoichiometric expression of the light chain and heavy chain of an antibody may improve expression of the resulting antibody, as improper ratios of the light chain and heavy chain can lead to potential aggregation and glycosylation of the monoclonal antibody Ho S C L et al., (May 2013), PLoS One. 21; 8(5):e63247. In some embodiments, the light chain and heavy chain of TARGT-antibody are produced in a stoichiometric fashion. There are a number of means of generating stoichiometric expression of proteins from a single cassette, and therefore the invention is not limited by the means by which the antibodies are expressed in a stoichiometric fashion.

In certain embodiments, the light chain and heavy chain sequences of an antibody are separated by an IRES sequence. Those skilled in the art would understand that there is a large range of IRES sequences, the list of which is diverse and constantly growing; therefore, the scope of the present invention is not limited by the particular IRES used within the construct. In some embodiments, the IRES is that contained within SEQ ID NO: 13. In other embodiments, the IRES is selected from known databases. The efficacy of any particular IRES element can be readily tested by detecting expression of the heavy and light chain using standard protocols. In certain embodiments, the antibody sequence upstream of the IRES contained a stop codon.

In some embodiments, the light chain and heavy chain sequences are separated by a 2A element or a 2A-like element. In certain embodiments, the 2A element is that of foot-and-mouth disease, as contained in SEQ ID NO: 12. In some embodiments, another 2A or 2A-like element is used. In certain embodiments, the 2A-like sequence is that from equine rhinitis A virus or thosea asigna virus. The efficacy of any particular 2A or 2A-like element can be readily tested by detecting expression of the heavy and light chain using standard protocols. In other embodiments, the construct does not contain a 2A element.

In certain embodiments, a furin cleavage sequence is upstream of the 2A element, to generate a furin 2A element (F2A) and eliminate the additional amino acids that would otherwise remain attached to the upstream protein after cleavage of the 2A element. In certain embodiments, the furin cleavage sequence is contained within SEQ ID: 11. In other embodiments, a pro-protein convertase other than furin is contained within the cassette. In some embodiments, the pro-protein convertase is one of PACE4, PC1/3, PC2, PC4, PC5/6, or PC7. In other embodiments, the construct does not contain a furin or other pro-protein cleavage site.

In certain embodiments, no method is employed to promote stoichiometric expression of the heavy and light chains by a TARGT.

6. Antibody Elements

Bispecific antibodies may be expressed in the micro-organs according to the recombinant techniques described herein. For example, the antibody elements of the cassettes may comprise a full length or partial length heavy and light chain of one antibody and a full length or partial length heavy and light chain of another antibody. The construct may be designed as follows: signal sequence, heavy chain, F2a, light chain, [(stop, IRES), or F2A] signal sequence, heavy chain, F2a, light chain, stop. Any length or variant of heavy and light chain sequences may be used as long as the bispecific antibody maintains binding to its two antigens.

Antibody fragments or variants thereof may lack the Fc region of intact antibody, clear more rapidly from the circulation, and may have less non-specific tissue binding than a control antibody containing an Fc region. Portions of antibodies may be made by expressing a portion of the recombinant molecule.

In one embodiment, the antibody may have an IgG, IgA, IgM, or IgE isotype. In one embodiment, the antibody is an IgG.

In some embodiments, the light chain and heavy chain sequences of an antibody are optimized. In certain embodiments, these optimized sequences are those of adalimumab and are contained within SEQ ID NO: 1-4. In other embodiments, the heavy and light chain sequences of a known antibody sequence are not optimized.

In some embodiments, the heavy chain sequence is downstream of the light chain sequence. In some embodiments, the light chain sequence is downstream of the heavy chain sequence. Those skilled in the art could test for differences in expression based on placements of different components within the expression cassette.

In one embodiment, the antibody or functional part thereof comprises a VH domain comprising a CDR1, a CDR2, and a CDR3, and a VL domain comprising a CDR1, a CDR2, and a CDR3.

In one embodiment, the micro-organ secretes an antibody or functional part thereof comprising a VH domain and a VL domain.

In certain embodiments, an antibody of the disclosure may immunospecifically bind to its target antigen and may have a dissociation constant (K_{d}) of less than about 3000 pM, less than about 2500 pM, less than about 2000 pM, less than about 1500 pM, less than about 1000 pM, less than about 750 pM, less than about 500 pM, less than about 250 pM, less than about 200 pM, less than about 150 pM, less than about 100 pM, less than about 75 pM as assessed using a method known to one of skill in the art (e.g., a BIAcore assay, ELISA) (Biacore International AB, Uppsala, Sweden).

7. Signal Sequences

In certain embodiments, the therapeutic protein sequence includes a signal sequence, which may be defined as a sequence of amino acids at the amino terminus. In certain embodiments, use of signal sequences (also known as signal peptides) may improve secretion of a therapeutic protein.

As there are a wide variety of signal sequences known to those skilled in the art, the invention is not limited by the specific signal sequence incorporated into the cassette. In certain embodiments, the signal sequences may be included from databases.

In certain embodiments, the light chain and heavy chain antibody sequences include a signal sequence. In certain embodiments, use of signal sequences (also known as signal peptides) may improve secretion of antibody. In other embodiments, the heavy chain signal sequence comprises an intron for stabilization, as noted in SEQ ID NO: 5. In some embodiments, the signal sequence is identical for the heavy chain and light chains, and in other embodiments the light and heavy chains contain different signal sequences. In one embodiment a heavy chain signal sequence is used in front of both the heavy chain and the light chain.

8. Polyadenylation Signals

In one embodiment, a polyadenylation site is used in the construct downstream of the therapeutic protein. In some embodiments, a polyadenylation site is used in the construct downstream of the heavy and light chain of an antibody. A number of polyadenylation signals would be known to those in the art to promote polyadenylation of an mRNA transcript, and any known sequence could be tested. In certain embodiments, the simian virus 40 (SV40) poly-adenylation signal is used, corresponding to SEQ ID NO: 10.

9. Tags

In one embodiment, the therapeutic protein produced by the micro-organ is flagged or tagged with a detectable moiety. The detectable moiety may be a fluorescent or enzymatic or other moiety that allows detection of the produced protein.

B. Freezing and Thawing of Micro-Organs

In some embodiments, micro-organs are harvested, transduced with a viral vector comprising a cassette encoding a therapeutic protein, and then frozen for later implantation in the CNS. In some embodiments, micro-organs are harvested, frozen, thawed, and then transduced with a viral vector comprising a cassette encoding a therapeutic protein. In some embodiments, multiple micro-organs may be harvested at the same time and then frozen for later use. In some embodiments, multiple micro-organs may be harvested and transduced at the same time and then frozen for later use.

In some embodiments, frozen micro-organs are thawed and cultured in vitro before being implanted in the CNS of the subject. In some embodiments, thawing of frozen micro-organs involves use of rinses with a pharmacologically inert buffer, such as saline. In some embodiments, thawing of frozen micro-organs involves use of serum previously collected from the subject, or commercially available serum compatible with the harvested micro-organ.

In some embodiments, micro-organs are not frozen before implantation. In some embodiments, micro-organs are harvested, transduced, cultured, and implanted into the CNS of the subject without being frozen.

1. Implantation Location of the Micro-Organ

Within this application, a “centrally implanted” or “CNS” micro-organ refers to a micro-organ which is implanted within the CNS. A location in the CNS could be any site within the brain or spinal cord. In some embodiments, the dermal micro-organ is implanted within the ventricular system of the brain. In some embodiments, the dermal micro-organ is implanted in the sub-dural space. In some embodiments, the dermal micro-organ is implanted using lumbar puncture (LP). In some embodiments, the dermal micro-organ is implanted in the spine, cisterna magna, ventricular system space of the brain, brain convexity, or brain parenchyma.

In some embodiments, the micro-organ is implanted at the same time as a procedure for biopsy, removal, or debulking of a CNS tumor. In some embodiments, the micro-organ is implanted at the same location where a CNS tumor is removed or debulked.

In some embodiments, the micro-organ secretes therapeutic protein directly into the cerebrospinal fluid (CSF). In some embodiments, levels of the therapeutic protein produced by the dermal micro-organ are measured in the CSF. In some embodiments, levels of the therapeutic protein produced by the micro-organ are measured following a spinal tap procedure to collect CSF. In some embodiments, levels of the therapeutic protein produced by the micro-organ are measured using a catheter that is implanted for the purpose of allowing periodic collection of CSF. In some embodiments, the catheter used to collect CSF is implanted at the same time or in the same procedure in which the dermal micro-organ is implanted. In some embodiments, the protein produced by the micro-organ contains a marker. In one embodiment, the marker is detectable. In some instances, the detectable marker comprises a radiolabel, a fluorescent marker, or an enzymatic label.

2. Secretion Levels

Surprisingly, the TARGT-CNS compositions of the invention secrete protein in the CNS for extended periods of time. For example, the TARGT-CNS compositions continue to secrete recombinant protein into the CNS for at least 2 years, 1 year, 11 months, 10 months, 9 months, 8 months, 7 months, 6 months, 5 months, 4 months, 3 months, 2 months, 1 month, 3 weeks, 2 weeks, 1 week, 6 days, 5 days, 4 days, 3 days, and 1 day.

In one embodiment, the TARGT-CNS compositions secrete recombinant protein into the serum, even when implanted in the CNS, thus implicating crossing of the blood brain barrier. The TARGT-CNS compositions are capable of secreting protein into the serum for at least 2 years, 1 year, 11 months, 10 months, 9 months, 8 months, 7 months, 6 months, 5 months, 4 months, 3 months, 2 months, 1 month, 3 weeks, 2 weeks, 1 week, 6 days, 5 days, 4 days, 3 days, and 1 day.

The secretion of the therapeutic protein is measurable in the CNS and also in the serum. In one embodiment, the therapeutic protein is measured at a site that is distant from the site of implantation.

C. Methods of Treatment and Prevention of Cancer in the CNS

Therapeutic proteins have efficacy in model systems for a variety of human diseases and conditions related to dysfunction or diseases of the CNS. Therefore, the therapeutic proteins produced by the TARGT-CNS compositions described herein are not limited by the nature of the disease/condition.

In certain embodiments, the therapeutic protein produced by the TARGT-CNS is for use in treatment of a cancer. In some embodiments, the cancer is primary to the CNS, meaning that the cancer originated in the CNS. In some embodiments, the cancer is secondary to the CNS, meaning that the cancer originated outside the CNS, but has spread to, or otherwise is having an effect on, the CNS. In some embodiments, the cancer manifests as a tumor in the CNS. In some embodiments, the cancer in the CNS is related to a tumor that is secondary to a primary tumor elsewhere in the body. In some embodiments, the cancer in the CNS is a metastasis. In some embodiments, the cancer in the CNS is a metastasis of colon, kidney, melanoma, lung, ovarian, breast, or testicular cancer.

In some embodiments, the cancer is an astrocytoma, glioblastoma, glioma, lymphoma, medulloblastoma, or CNS lymphoma. The TARGT-CNS is administered to the CNS of the patient to treat the cancer.

Likewise, methods of treating cancer are encompassed, comprising administering/implanting a TARGT-CNS composition to the CNS, wherein the TARGT-CNS secretes a therapeutically relevant amount of protein to effectively treat the cancer.

In some embodiments, it is unclear whether the cancer has a source in the CNS or periphery. Treatment of a tumor or malignancy in the CNS by this invention is not limited by the source of the tumor or malignancy. As such, any tumor or malignancy with a location within the CNS would fall within the definition of “cancer within the CNS” or “CNS cancer.”

1. Combination Therapy

In some embodiments, treatment with a TARGT-CNS is combined with another therapy. In some embodiments, combination treatment is for the purpose of promoting extended viability of the micro-organ. In some embodiments, treatment with a TARGT-CNS is combined with a steroid or other immunosuppressant. In some embodiments, this additional immunosuppressive therapy is administered to the CNS. In some embodiments, this additional immunosuppressive therapy is administered peripherally.

In some embodiments, treatment with a TARGT-CNS is combined with peripheral therapy. In some embodiments, treatment with a TARGT-CNS provides delivery of the therapeutic protein to the CNS, while peripheral therapy would provide peripheral (non-CNS) delivery of the same or similar therapeutic protein. In some embodiments, TARGT-mediated therapy may be mediated by a centrally implanted micro-organ(s), in addition to micro-organ(s) implanted at a peripheral location. In some embodiments, a TARGT-CNS may be used in combination with a peripheral that is not mediated by a TARGT.

In some embodiments, treatment with a TARGT-CNS is combined with another chemotherapeutic therapy. In some embodiments, this additional chemotherapeutic therapy is administered centrally. In some embodiments, this additional chemotherapeutic therapy is administered peripherally. In some embodiments, this additional chemotherapeutic agent is a biologic agent. In some embodiments, this biologic agent is an antibody. In some embodiments, this additional chemotherapeutic agent is a non-biologic agent. In some embodiments, this additional chemotherapeutic agent is an alkylating agent, antimetabolite, anti-tumor antibiotic, topomerase inhibitor, or mitotic inhibitor. A wide range of chemotherapeutic agents would be known to practicing clinicians, and an additional chemotherapeutic agent may be any approved or experimental agent with an indication for treatment or prevention of recurrence of any cancer.

D. Methods of Treatment and Prevention of Lysosomal Storage Diseases in the CNS

In certain embodiments, the therapeutic protein produced by the TARGT-CNS is for use in treatment of genetic disorders involving the CNS. In some embodiments, the genetic disorder is caused by the lack of expression of a gene product. In some embodiments, the genetic disorder caused by the improper expression of a gene product such as lower levels of gene product. In some embodiments, the genetic disorder is caused by misexpression of a gene product. Misexpression would include any mutation leading to misfolding, mistrafficking, degradation, or either defects in the gene product.

In some embodiments, the genetic disorder is one in which the CNS is a primary site of symptoms. In some embodiments, the genetic disorder is one in which defects in a gene product produce symptoms in a number of areas, including the CNS.

In some embodiments, expression of a therapeutic protein by TARGT-CNS replaces a missing gene product or improperly expressed gene product. In some embodiments, the missing gene product or improperly expressed gene product is caused by a genetic disorder characterized by a mutation in the subject's genome.

In some embodiments, the genetic disorder treated is a lysosomal storage disease. A lysosomal storage disease is any disease characterized by deficiency of an enzyme. As such, any disease related to deficiency of an enzyme would be defined as a lysosomal storage disease. As new mutations and rare diseases are being described, diseases not listed herein or presently described in the medical literature, but which are found to involve deficiency in an enzyme, would be included in the definition of a lysosomal storage disease. In some embodiments, the lysosomal storage disease treated is Hunter disease, Fabry disease, infantile Batten disease (CNL1), classic late infantile Batten disease (CNL2), Hurler syndrome, Krabbe disease, Niemann-Pick (including A and C forms of the disease), and Pompe disease.

In some embodiments, the therapeutic protein expressed by the micro-organ replaces a gene product that is not an enzyme. In some embodiments, the therapeutic protein expressed by the micro-organ replaces a gene product that does not catalyze a reaction in the CNS.

In certain embodiments, the micro-organ may express a therapeutic protein that is normally produced in the CNS. In some embodiments, the micro-organ may express a therapeutic protein that is not normally produced in the CNS, such as a therapeutic antibody.

1. Combination Therapy

In some embodiments, treatment with a TARGT-CNS is combined with another therapy. In some embodiments, treatment with a TARGT-CNS is combined with another agent for the purpose of promoting extended viability of the micro-organ. In some embodiments, treatment with a TARGT-CNS is combined with a steroid or other immunosuppressant. In some embodiments, this additional immunosuppressive therapy is administered centrally. In some embodiments, this additional immunosuppressive therapy is administered peripherally.

In some embodiments, treatment with a TARGT-CNS is combined with peripheral replacement therapy. In some embodiment, treatment with a TARGT-CNS provides delivery of the therapeutic protein to the CNS, while peripheral replacement therapy would provide peripheral delivery of the same or similar therapeutic protein. In some embodiments, TARGT-mediated therapy may be mediated by a centrally implanted micro-organ(s), in addition to micro-organ(s) implanted at a peripheral location. In some embodiments, a TARGT-CNS may be used in combination with a peripheral enzyme replacement that is not mediated by a TARGT.

In some embodiments, a TARGT-CNS is used in combination with substrate reduction therapy. In some embodiments, a TARGT-CNS is used in combination with a means to reduce the formation of a lysosomal substance.

In some embodiments, a TARGT-CNS is used as a maintenance therapy while a suitable donor is found for a subject to undergo a bone marrow transplantation.

In some embodiments, treatment with a TARGT-CNS for a lysosomal storage disease is not combined with any other therapy.

E. Dosing

The variables of the dosing schedule will be determined by one of skill in the art depending on the disorder being treated and choice of treatment. For example, for chronic conditions, such as genetic disorders, TARGT-CNS transplantation may occur with more regular frequency. In some embodiments, the level of therapeutic protein produced by the TARGT-CNS in the cerebrospinal fluid (CSF) determines the timing of subsequent implantations or removal of dermal micro-organs. In some embodiments, the levels of therapeutic protein produced by the micro-organ in vitro is used to determine the number that are implanted into a subject.

In some embodiments, the therapeutic protein produced by the TARGT-CNS is prophylactic or preventative. In certain embodiments, the TARGT-CNS may be implanted before symptoms of a disease are apparent, such as a patient diagnosed with a genetic disorder based on a family history or sequencing or similar genetic screen, but who does not yet have any symptoms.

In some embodiments, the therapeutic protein produced by the TARGT-CNS is intended for short-term treatment. In certain embodiments, a measure of disease activity is used to determine when treatment with the TARGT-CNS has been successful. In certain embodiments, the micro-organ is removed when measures of disease activity indicate that treatment with therapeutic protein from a micro-organ is no longer necessary, and the micro-organ can be removed. In certain embodiments, regression of a tumor may be the measure of disease activity that indicates that treatment with therapeutic protein from a micro-organ is no longer necessary, and the micro-organ can be removed.

In some embodiments, measures of the therapeutic protein in the CSF produced by the micro-organ are used to determine the optimal number of micro-organs to be implanted. In some embodiments, micro-organs secreting therapeutic protein may be removed or added based on measures of the therapeutic protein in the CSF produced by the micro-organ.

In some embodiments, measures of disease activity are used to determine the optimal number of micro-organs to be used. In some embodiments, micro-organs secreting therapeutic protein may be removed or added based on measures of disease activity. In certain embodiments, the measures of disease activity to determine the optimal number of micro-organs may be tumor size, levels of disease biomarkers, or any other diagnostic of disease activity that may come, for example, from imaging, blood work, or other diagnostics known to those skilled in the art.

In certain embodiments, a subject undergoing combination therapy can receive both TARGT-protein and additional agent at the same time (e.g., simultaneously) or at different times (e.g., sequentially, in either order, on the same day, or on different days), so long as the therapeutic effect of the combination of both substances is caused in the subject undergoing therapy. In some embodiments, the combination of TARGT-protein and additional agent will be given simultaneously. Sequential administration may be performed regardless of whether the subject responds to the first administration.

### DESCRIPTION OF THE SEQUENCES

This table provides a listing of certain sequences referenced herein.

| | | SEQ ID |
|---|---|---|
| Description | Sequences | NO |
| Optimized | GACATCCAAA TGACACAGAG TCCTTCCTCC | 1 |
| light chain | TTGTCAGCTA GTGTTGGAGA CCGCGTTACT | |
| sequence | ATCACATGCA GGGCGTCACA AGGCATCAGG | |
| from | AATTACTTGG CGTGGTACCA GCAGAAGCCT | |
| adalimumab | GGAAAAGCCC CAAAACTGCT GATATACGCA | |
| contained | GCCAGCACAC TTCAATCAGG CGTGCCCTCT | |
| in TNF1 | AGGTTCTCTG GCTCCGGTTC CGGAACCGAC | |
| construct | TTCACACTCA CCATATCCTC ACTGCAACCT | |
| | GAAGACGTGG CCACATACTA TTGTCAGCGC | |
| | TATAATAGGG CACCCTACAC TTTTGGCCAA | |
| | GGGACGAAAG TGGAAATAAA AAGGACAGTG | |
| | GCAGCTCCGT CCGTTTTTAT CTTCCCTCCA | |
| | TCCGATGAGC AGCTTAAGTC TGGGACTGCT | |
| | TCCGTAGTGT GTTTGCTGAA TAATTTTTAT | |
| | CCCCGAGAAG CAAAGGTTCA GTGGAAGGTC | |
| | GATAATGCCC TGCAGAGTGG CAATAGTCAG | |
| | GAGTCCGTAA CCGAGCAGGA CTCTAAGGAC | |
| | TCCACCTATT CCCTGAGTTC CACCTTGACC | |
| | CTTTCCAAGG CCGACTATGA GAAGCACAAA | |
| | GTATACGCCT GCGAGGTAAC TCACCAGGGA | |
| | TTGAGCTCCC CAGTGACAAA GTCATTTAAT | |
| | CGGGGCGAGT GCCTGTCCAA GGCCGACTAC | |
| | GAAAAGCACA AAGTGTACGC CTGTGAAGTC | |
| | ACCCATCAGG GCCTGTCATC TCCAGTCACG | |
| | AAGTCATTCA ATCGAGGGGA GTGC | |
| Optimized | GACATCCAGA TGACGCAGTC CCCAAGCTCA | 2 |
| light chain | CTGTCCGCCT CTGTAGGTGA CCGGGTAACT | |
| sequence | ATCACCTGCA GAGCATCCCA GGGCATCCGC | |
| from | AATTACCTGG CCTGGTATCA GCAGAAACCT | |
| adalimumab | GGCAAGGCCC CAAAACTCCT CATCTACGCA | |
| contained | GCATCCACCC TTCAGAGTGG CGTACCAAGC | |
| in TNF3 | CGATTCTCCG GAAGCGGTAG TGGAACCGAC | |
| construct | TTTACCCTCA CAATCTCAAG TCTGCAGCCT | |
| | GAAGATGTCG CTACATATTA TTGCCAGAGA | |
| | TACAATAGGG CCCCATACAC CTTTGGGCAG | |
| | GGCACGAAAG TGGAAATTAA GCGCACAGTT | |
| | GCGGCACCAA GTGTGTTTAT TTTCCCGCCC | |
| | AGCGATGAAC AGCTGAAATC CGGCACGGCC | |
| | AGCGTTGTAT GCTTGCTGAA TAACTTTTAC | |
| | CCTAGAGAGG CCAAGGTCCA ATGGAAGGTT | |
| | GACAACGCAC TGCAGTCCGG CAACAGTCAA | |
| | GAGAGCGTCA CTGAACAAGA TTCCAAGGAC | |
| | AGTACATACT CACTCAGCTC CACACTGACA | |
| | CTCTCCAAGG CCGACTACGA GAAGCATAAG | |
| | GTCTACGCTT GCGAGGTAAC GCATCAGGGC | |
| | CTTTCTAGCC CAGTTACCAA AAGTTTCAAT | |
| | CGAGGCGAAT GCCTGTCAAA AGCAGACTAC | |
| | GAGAAACACA AGGTTTACGC CTGTGAAGTG | |
| | ACACACCAGG GCTTGAGCTC CCCTGTGACA | |
| | AAATCTTTTA ATAGGGGAGA GTGT | |
| Optimized | GAAGTGCAGC TTGTGGAGTC TGGCGGTGGC | 3 |
| heavy chain | CTCGTGCAGC CAGGCCGGAG CCTGCGGCTG | |
| sequence | AGCTGTGCAG CCAGCGGGTT CACCTTCGAT | |
| from | GATTATGCTA TGCACTGGGT TCGCCAGGCC | |
| adalimumab | CCCGGAAAGG GCCTGGAGTG GGTCTCAGCT | |
| contained | ATCACATGGA ATTCCGGACA CATCGACTAC | |
| in TNF1 | GCCGACAGCG TGGAGGGGCG CTTTACCATT | |
| construct | TCAAGGGACA ACGCTAAAAA CAGCCTGTAC | |
| | CTTCAGATGA ACTCCCTGCG GGCGGAAGAC | |
| | ACAGCGGTGT ACTACTGTGC CAAGGTGAGC | |
| | TACCTGTCCA CAGCATCCTC ATTGGACTAT | |
| | TGGGGCCAAG GCACGCTGGT TACCGTTTCC | |
| | AGCGCAAGCA CAAAGGGACC TAGTGTGTTC | |
| | CCGTTGGCCC CTTCAAGCAA ATCCACGAGT | |
| | GGAGGCACCG CTGCACTGGG CTGCCTTGTA | |
| | AAGGACTACT TCCCGGAGCC AGTGACTGTG | |
| | TCATGGAACA GTGGCGCCCT GACAAGCGGA | |
| | GTCCACACTT TTCCTGCGGT CCTCCAGTCC | |
| | TCCGGGCTTT ACAGCCTGAG TAGTGTGGTT | |
| | ACCGTCCCCT CATCCTCCCT GGGTACCCAG | |
| | ACCTACATTT GTAATGTGAA CCATAAGCCA | |
| | AGCAATACAA AGGTGGATAA AAAGGTGGAG | |
| | CCAAAAAGCT GCGATAAAAC ACATACTTGC | |
| | CCTCCTTGCC CAGCGCCCGA GTTGCTCGGC | |
| | GGCCCTTCCG TATTTCTTTT TCCACCGAAA | |
| | CCGAAGGATA CACTGATGAT CTCTCGGACC | |
| | CCTGAGGTCA CTTGTGTGGT GGTTGACGTT | |
| | TCACACGAGG ACCCAGAAGT GAAGTTTAAT | |
| | TGGTACGTGG ATGGGGTTGA GGTGCACAAT | |
| | GCTAAAACCA AGCCGCGCGA GGAGCAATAT | |
| | AACTCTACCT ATCGAGTGGT GAGCGTGCTC | |
| | ACCGTACTCC ATCAGGACTG GCTGAACGGG | |
| | AAGGAGTACA AGTGCAAGGT TTCAAACAAG | |
| | GCTCTCCCTG CCCCAATAGA GAAGACCATA | |
| | AGTAAAGCCA AGGGACAGCC TCGCGAGCCA | |
| | CAGGTCTATA CTCTGCCTCC TAGTAGGGAC | |
| | GAGCTCACCA AGAACCAGGT AAGCCTCACC | |
| | TGCTTGGTCA AGGGCTTTTA TCCATCCGAC | |
| | ATCGCCGTGG AATGGGAGAG CAACGGACAG | |
| | CCTGAAAACA ACTACAAAAC TACCCCACCC | |
| | GTTCTTGATT CAGATGGGAG CTTTTTTCTG | |
| | TACAGCAAGT TGACCGTCGA TAAATCCCGA | |
| | TGGCAGCAGG GAAATGTTTT CTCTTGCTCA | |
| | GTGATGCATG AAGCGCTGCA CAACCACTAT | |
| | ACACAGAAGA GCCTTAGCTT GTCTCCAGGA AAA | |
| Optimized | GAAGTGCAGT TGGTCGAGTC CGGTGGAGGG | 4 |
| heavy chain | CTGGTCCAGC CTGGCAGAAG TCTCCGGCTG | |
| sequence | AGTTGCGCAG CCAGCGGATT CACCTTCGAC | |
| from | GATTACGCCA TGCACTGGGT GCGGCAGGCC | |
| adalimumab | CCGGGCAAGG GCCTTGAATG GGTGTCTGCG | |
| contained | ATCACATGGA ATTCCGGACA TATTGATTAC | |
| in TNF3 | GCCGACAGCG TGGAGGGCCG ATTCACCATC | |
| construct | AGTAGGGATA ATGCTAAGAA CTCCCTGTAC | |
| | CTGCAGATGA ATAGTCTGAG GGCTGAAGAC | |
| | ACAGCCGTGT ACTATTGCGC AAAAGTCAGC | |
| | TACCTCTCCA CTGCTTCTAG TCTGGACTAC | |
| | TGGGGTCAGG GGACGCTGGT GACGGTTTCT | |
| | TCCGCATCCA CTAAAGGTCC TAGCGTTTTC | |
| | CCCCTCGCCC CCTCTTCTAA GAGCACCTCC | |
| | GGAGGAACTG CAGCCCTTGG ATGCTTGGTT | |
| | AAAGATTACT TTCCCGAACC CGTAACCGTA | |
| | AGCTGGAACA GTGGCGCCCT GACTTCAGGG | |
| | GTACACACCT TTCCGGCCGT GCTGCAGAGC | |
| | AGCGGGCTCT ATAGCCTTAG CTCAGTCGTG | |
| | ACGGTCCCAT CCTCTAGTCT TGGTACTCAA | |
| | ACCTACATCT GCAATGTGAA TCACAAGCCT | |
| | TCTAACACAA AAGTTGATAA GAAAGTAGAA | |
| | CCCAAGAGCT GTGATAAGAC ACATACTTGT | |
| | CCTCCCTGTC CGGCCCCCGA ATTGCTTGGG | |
| | GGGCCGAGTG TCTTCCTCTT CCCTCCAAAA | |
| | CCCAAGGACA CTCTCATGAT TTCAAGGACC | |
| | CCTGAAGTGA CTTGTGTGGT AGTTGACGTG | |
| | AGCCACGAGG ACCCTGAAGT GAAGTTCAAT | |
| | TGGTATGTGG ATGGCGTTGA GGTGCATAAT | |
| | GCAAAGACAA AGCCACGCGA GGAGCAGTAC | |
| | AATTCCACCT ATAGGGTGGT ATCCGTGCTG | |
| | ACCGTGTTGC ATCAGGACTG GCTCAATGGG | |
| | AAAGAGTATA AATGTAAGGT GTCCAATAAG | |
| | GCCCTGCCCG CTCCCATTGA AAAAACAATT | |
| | TCAAAGGCTA AGGGCCAACC CCGCGAACCA | |
| | CAAGTCTACA CACTCCCCCC TAGTAGAGAT | |
| | GAGCTGACAA AAAATCAGGT GTCTCTCACA | |
| | TGTCTGGTAA AAGGCTTCTA TCCTTCAGAT | |
| | ATTGCTGTGG AATGGGAATC AAATGGGCAG | |
| | CCAGAGAATA ACTACAAAAC GACACCCCCA | |
| | GTCCTTGATA GTGACGGGTC CTTCTTCCTC | |
| | TACTCTAAAC TCACCGTGGA CAAGAGTAGA | |
| | TGGCAACAGG GCAATGTGTT CTCCTGTAGC | |
| | GTCATGCATG AAGCACTGCA CAATCATTAT | |
| | ACTCAGAAGA GCTTGTCCCT TAGTCCAGGA AAA | |
| Heavy chain | GGATGGAGCT GTATCATCCT CTTCTTGGTA | 5 |
| signal | GCAACAGCTA CAGGTAAGGG GTTAACAGTA | |
| sequence | GCAGGCTTGA GGTCTGGACA TATATATGGG | |
| containing | TGACAATGAC ATCCACTTTG CCTTTCTCTC | |
| intron | CACAGGCGCG CACTCC | |
| CMV | GAGTCAATGG GAAAAACCCA TTGGAGCCAA | 6 |
| enhancer | GTACACTGAC TCAATAGGGA CTTTCCATTG | |
| | GGTTTTGCCC AGTACATAAG GTCAATAGGG | |
| | GGTGAGTCAA CAGGAAAGTC CCATTGGAGC | |
| | CAAGTACATT GAGTCAATAG GGACTTTCCA | |
| | ATGGGTTTTG CCCAGTACAT AAGGTCAATG | |
| | GGAGGTAAGC CAATGGGTTT TTCCCATTAC | |
| | TGACATGTAT ACTGAGTCAT TAGGGACTTT | |
| | CCAATGGGTT TTGCCCAGTA CATAAGGTCA | |
| | ATAGGGGTGA ATCAACAGGA AAGTCCCATT | |
| | GGAGCCAAGT ACACTGAGTC AATAGGGACT | |
| | TTCCATTGGG TTTTGCCCAG TACAAAAGGT | |
| | CAATAGGGGG TGAGTCAATG GGTTTTTCCC | |
| | ATTATTGGCA CATACATAAG GTCAATAGGG GTG | |
| EF1α | ACTAGTGGAG AAGAGCATGC TTGAGGGCTG | 7 |
| promoter | AGTGCCCCTC AGTGGGCAGA GAGCACATGG | |
| | CCCACAGTCC CTGAGAAGTT GGGGGGAGGG | |
| | GTGGGCAATT GAACTGGTGC CTAGAGAAGG | |
| | TGGGGCTTGG GTAAACTGGG AAAGTGATGT | |
| | GGTGTACTGG CTCCACCTTT TTCCCCAGGG | |
| | TGGGGGAGAA CCATATATAA GTGCAGTAGT | |
| | CTCTGTGAAC ATTC | |
| CpG-free | TTAATTAAAA TTATCTCTAA GGCATGTGAA | 8 |
| MAR from | CTGGCTGTCT TGGTTTTCAT CTGTACTTCA | |
| human β- | TCTGCTACCT CTGTGACCTG AAACATATTT | |
| globin gene | ATAATTCCAT TAAGCTGTGC ATATGATAGA | |
| | TTTATCATAT GTATTTTCCT TAAAGGATTT | |
| | TTGTAAGAAC TAATTGAATT GATACCTGTA | |
| | AAGTCTTTAT CACACTACCC AATAAATAAT | |
| | AAATCTCTTT GTTCAGCTCT CTGTTTCTAT | |
| | AAATATGTAC CAGTTTTATT GTTTTTAGTG | |
| | GTAGTGATTT TATTCTCTTT CTATATATAT | |
| | ACACACACAT GTGTGCATTC ATAAATATAT | |
| | ACAATTTTTA TGAATAAAAA ATTATTAGCA | |
| | ATCAATATTG AAAACCACTG ATTTTTGTTT | |
| | ATGTGAGCAA ACAGCAGATT AAAAGGCTAG | |
| | CCTGCAG | |
| MAR 5′ | AGTCAATATG TTCACCCCAA AAAAGCTGTT | 9 |
| region from | TGTTAACTTG CCAACCTCAT TCTAAAATGT | |
| human IFN- | ATATAGAAGC CCAAAAGACA ATAACAAAAA | |
| beta gene | TATTCTTGTA GAACAAAATG GGAAAGAATG | |
| | TTCCACTAAA TATCAAGATT TAGAGCAAAG | |
| | CATGAGATGT GTGGGGATAG ACAGTGAGGC | |
| | TGATAAAATA GAGTAGAGCT CAGAAACAGA | |
| | CCCATTGATA TATGTAAGTG ACCTATGAAA | |
| | AAAATATGGC ATTTTACAAT GGGAAAATGA | |
| | TGGTCTTTTT CTTTTTTAGA AAAACAGGGA | |
| | AATATATTTA TATGTAAAAA ATAAAAGGGA | |
| | ACCCATATGT CATACCATAC ACACAAAAAA | |
| | ATTCCAGTGA ATTATAAGTC TAAATGGAGA | |
| | AGGCAAAACT TTAAATCTTT TAGAAAATAA | |
| | TATAGAAGCA TGCCATCAAG ACTTCAGTGT | |
| | AGAGAAAAAT TTCTTATGAC TCAAAGTCCT | |
| | AACCACAAAG AAAAGATTGT TAATTAGATT | |
| | GCATGAATAT TAAGACTTAT TTTTAAAATT | |
| | AAAAAACCAT TAAGAAAAGT CAGGCCATAG | |
| | AATGACAGAA AATATTTGCA ACACCCCAGT | |
| | AAAGAGAATT GTAATATGCA GATTATAAAA | |
| | AGAAGTCTTA CAAATCAGTA AAAAATAAAA | |
| | CTAGACAAAA ATTTGAACAG ATGAAAGAGA | |
| | AACTCTAAAT AATCATTACA CATGAGAAAC | |
| | TCAATCTCAG AAATCAGAGA ACTATCATTG | |
| | CATATACACT AAATTAGAGA AATATTAAAA | |
| | GGCTAAGTAA CATCTGTGGC TTAATTAA | |
| SV40 | CCAGACATGA TAAGATACAT TGATGAGTTT | 10 |
| polyaden- | GGACAAACCA CAACTAGAAT GCAGTGAAAA | |
| ylation | AAATGCTTTA TTTGTGAAAT TTGTGATGCT | |
| signal | ATTGCTTTAT TTGTAACCAT TATAAGCTGC | |
| | AATAAACAAG TTAACAACAA CAATTGCATT | |
| | CATTTTATGT TTCAGGTTCA GGGGGAGGTG | |
| | TGGGAGGTTT TTTAAAGCAA GTAAAACCTC | |
| | TACAAATGTG GTATGGAATT C | |
| Furin | CGGGCAAAAC GG | 11 |
| sequence | | |
| 2A sequence | GCTCCCGTTA AACAGACGCT GAATTTCGAT | 12 |
| | CTCCTGAAGT TGGCCGGAGA CGTCGAATCA | |
| | AACCCCGGCC CA | |
| IRES | ATGATAATAT GGCCACAACC ATG | 13 |
| sequence | | |
| Sequence of | GTTGGTGTAC AGTAGTAGCA AGCTTGCATG | 14 |
| the TNF1 | CCTGCAGGTC GACTCTAGAC TGCCatgGGA | |
| construct | TGGAGCTGTA TCATCCTCTT CTTGGTAGCA | |
| | 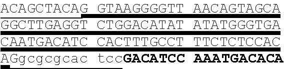 | |
| | GAGTCCTTCC TCCTTGTCAG CTAGTGTTGG | |
| | AGACCGCGTT ACTTCACATG CAGGGCGTCA | |
| | CAAGGCATCA GGAATTACTT GGCGTGGTAC | |
| | CAGCAGAAGC CTGGAAAAGC CCCAAAACTG | |
| | CTGATATACG CAGCCAGCAC ACTTCAATCA | |
| | GGCGTGCCCT CTAGGTTCTC TGGCTCCGGT | |
| | TCCGGAACCG ACTTCACACT CACCATATCC | |
| | TCACTGCAAC CTGAAGACGT GGCCACATAC | |
| | TATTGTCAGC GCTATAATAG GGCACCCTAC | |
| | ACTTTTGGCC AAGGGACGAA AGTGGAAATA | |
| | AAAAGGACAG TGGCAGCTCC GTCCGTTTTT | |
| | ATCTTCCCTC CATCCGATGA GCAGCTTAAG | |
| | TCTGGGACTG CTTCCGTAGT GTGTTTGCTG | |
| | AATAATTTTT ATCCCCGAGA AGCAAAGGTT | |
| | CAGTGGAAGG TCGATAATGC CCTGCAGAGT | |
| | GGCAATAGTC AGGAGTCCGT AACCGAGCAG | |
| | GACTCTAAGG ACTCCACCTA TTCCCTGAGT | |
| | TCCACCTTGA CCCTTTCCAA GGCCGACTAT | |
| | GAGAAGCACA AAGTATACGC CTGCGAGGTA | |
| | ACTCACCAGG GATTGAGCTC CCCAGTGACA | |
| | AAGTCATTTA ATCGGGGCGA GTGCCTGTCC | |
| | AAGGCCGACT ACGAAAAGCA CAAAGTGTAC | |
| | GCCTGTGAAG TCACCCATCA GGGCCTGTCA | |
| | TCTCCAGTCA CGAAGTCATT CAATCGAGGG | |
| | 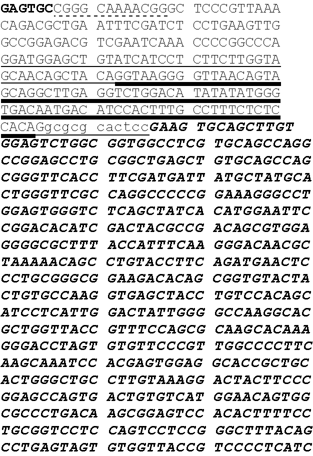 | |
| | 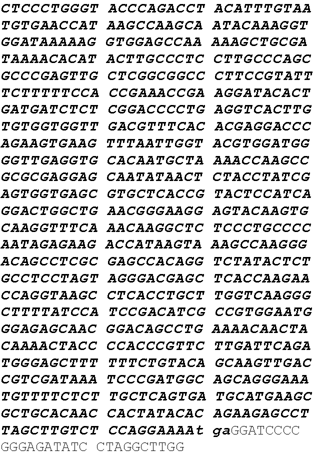 | |
| | 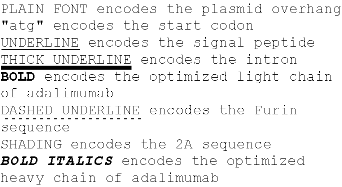 | |
| Sequence of | GTTGGTGTAC AGTAGTAGCA AGCTTGCATG | 15 |
| the TNF3 | CCTGCAGGTC GACTCTAGAC TGCCatgGGa | |
| construct | 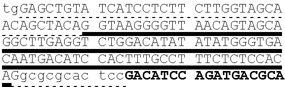 | |
| | GTCCCCAAGC TCACTGTCCG CCTCTGTAGG | |
| | TGACCGGGTA ACTATCACCT GCAGAGCATC | |
| | CCAGGGCATC CGCAATTACC TGGCCTGGTA | |
| | TCAGCAGAAA CCTGGCAAGG CCCCAAAACT | |
| | CCTCATCTAC GCAGCATCCA CCCTTCAGAG | |
| | TGGCGTACCA AGCCGATTCT CCGGAAGCGG | |
| | TAGTGGAACC GACTTTACCC TCACAATCTC | |
| | AAGTCTGCAG CCTGAAGATG TCGCTACATA | |
| | TTATTGCCAG AGATACAATA GGGCCCCATA | |
| | CACCTTTGGG CAGGGCACGA AAGTGGAAAT | |
| | TAAGCGCACA GTTGCGGCAC CAAGTGTGTT | |
| | TATTTTCCCG CCCAGCGATG AACAGCTGAA | |
| | ATCCGGCACG GCCAGCGTTG TATGCTTGCT | |
| | GAATAACTTT TACCCTAGAG AGGCCAAGGT | |
| | CCAATGGAAG GTTGACAACG CACTGCAGTC | |
| | CGGCAACAGT CAAGAGAGCG TCACTGAACA | |
| | AGATTCCAAG GACAGTACAT ACTCACTCAG | |
| | CTCCACACTG ACACTCTCCA AGGCCGACTA | |
| | CGAGAAGCAT AAGGTCTACG CTTGCGAGGT | |
| | AACGCATCAG GGCCTTTCTA GCCCAGTTAC | |
| | CAAAAGTTTC AATCGAGGCG AATGCCTGTC | |
| | AAAAGCAGAC TACGAGAAAC ACAAGGTTTA | |
| | CGCCTGTGAA GTGACACACC AGGGCTTGAG | |
| | CTCCCCTGTG ACAAAATCTT TTAATAGGGG | |
| | AGAGTGTtga ATGATAATAT GGCCACAACC | |
| | 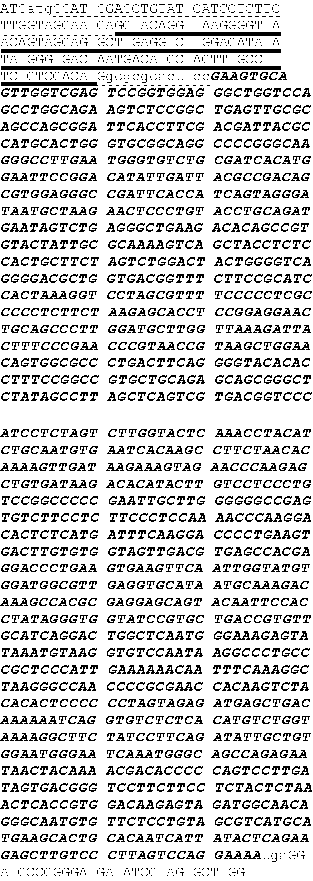 | |
| | 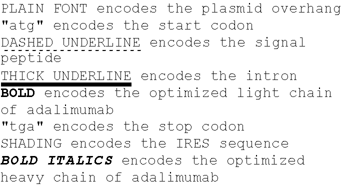 | |
| Sequence of | GGCCGATTCA TTAATGCAGG GGCCGCTGCG | 16 |
| pAd-MAR- | GCCATCATCA ATAATATACC TTATTTTGGA | |
| EF1a-opt | TTGAAGCCAA TATGATAATG AGGGGGTGGA | |
| hTNF1 | GTTTGTGACG TGGCGCGGGG CGTGGGAACG | |
| | GGGCGGGTGA CGTAGTAGTG TGGCGGAAGT | |
| | GTGATGTTGC AAGTGTGGCG GAACACATGT | |
| | AAGCGACGGA TGTGGCAAAA GTGACGTTTT | |
| | TGGTGTGCGC CGGTGTACAC AGGAAGTGAC | |
| | AATTTTCGCG CGGTTTTAGG CGGATGTTGT | |
| | AGTAAATTTG GGCGTAACCG AGTAAGATTT | |
| | GGCCATTTTC GCGGGAAAAC TGAATAAGAG | |
| | GAAGTGAAAT CTGAATAATT TTGTGTTACT | |
| | CATAGCGCGT AATATTTGTC TAGGGCCGCG | |
| | GGGACTTTGA CCGTTTACGT GGAGACTCGC | |
| | CCAGGTGTTT TTCTCAGGTG TTTTCCGCGT | |
| | TCCGGGTCAA AGTTGGCGTT TTATTATTAT | |
| | AGTCAGCTGA CGTGTAGTGT ATTTATACCC | |
| | GGTGAGTTCC TCAAGAGGCC ACTCTTGAGT | |
| | GCCAGCGAGT AGAGTTTTCT CCTCCGAGCC | |
| | GCTCCGACAC CGGGAGGCGC GCCTTAATTA | |
| | AAATTATCTC TAAGGCATGT GAACTGGCTG | |
| | TCTTGGTTTT CATCTGTACT TCATCTGCTA | |
| | CCTCTGTGAC CTGAAACATA TTTATAATTC | |
| | CATTAAGCTG TGCATATGAT AGATTTATCA | |
| | TATGTATTTT CCTTAAAGGA TTTTTGTAAG | |
| | AACTAATTGA ATTGATACCT GTAAAGTCTT | |
| | TATCACACTA CCCAATAAAT AATAAATCTC | |
| | TTTGTTCAGC TCTCTGTTTC TATAAATATG | |
| | TACCAGTTTT ATTGTTTTTA GTGGTAGTGA | |
| | TTTTATTCTC TTTCTATATA TATACACACA | |
| | CATGTGTGCA TTCATAAATA TATACAATTT | |
| | TTATGAATAA AAAATTATTA GCAATCAATA | |
| | TTGAAAACCA CTGATTTTTG TTTATGTGAG | |
| | CAAACAGCAG ATTAAAAGGC TAGCCTGCAG | |
| | GAGTCAATGG GAAAAACCCA TTGGAGCCAA | |
| | GTACACTGAC TCAATAGGGA CTTTCCATTG | |
| | GGTTTTGCCC AGTACATAAG GTCAATAGGG | |
| | GGTGAGTCAA CAGGAAAGTC CCATTGGAGC | |
| | CAAGTACATT GAGTCAATAG GGACTTTCCA | |
| | ATGGGTTTTG CCCAGTACAT AAGGTCAATG | |
| | GGAGGTAAGC CAATGGGTTT TTCCCATTAC | |
| | TGACATGTAT ACTGAGTCAT TAGGGACTTT | |
| | CCAATGGGTT TTGCCCAGTA CATAAGGTCA | |
| | ATAGGGGTGA ATCAACAGGA AAGTCCCATT | |
| | GGAGCCAAGT ACACTGAGTC AATAGGGACT | |
| | TTCCATTGGG TTTTGCCCAG TACAAAAGGT | |
| | CAATAGGGGG TGAGTCAATG GGTTTTTCCC | |
| | ATTATTGGCA CATACATAAG GTCAATAGGG | |
| | 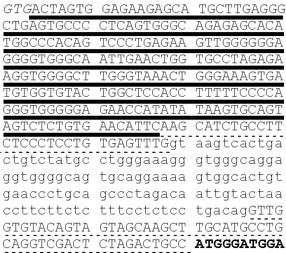 | |
| | GCTGTATCAT CCTCTTCTTG GTAGCAACAG | |
| | CTACAGGTAA GGGGTTAACA GTAGCAGGCT | |
| | TGAGGTCTGG ACATATATAT GGGTGACAAT | |
| | GACATCCACT TTGCCTTTCT CTCCACAGgc | |
| | gcgcactccG ACATCCAAAT GACACAGAGT | |
| | CCTTCCTCCT TGTCAGCTAG TGTTGGAGAC | |
| | CGCGTTACTA TCACATGCAG GGCGTCACAA | |
| | GGCATCAGGA ATTACTTGGC GTGGTACCAG | |
| | CAGAAGCCTG GAAAAGCCCC AAAACTGCTG | |
| | ATATACGCAG CCAGCACACT TCAATCAGGC | |
| | GTGCCCTCTA GGTTCTCTGG CTCCGGTTCC | |
| | GGAACCGACT TCACACTCAC CATATCCTCA | |
| | CTGCAACCTG AAGACGTGGC CACATACTAT | |
| | TGTCAGCGCT ATAATAGGGC ACCCTACACT | |
| | TTTGGCCAAG GGACGAAAGT GGAAATAAAA | |
| | AGGACAGTGG CAGCTCCGTC CGTTTTTATC | |
| | TTCCCTCCAT CCGATGAGCA GCTTAAGTCT | |
| | GGGACTGCTT CCGTAGTGTG TTTGCTGAAT | |
| | AATTTTTATC CCCGAGAAGC AAAGGTTCAG | |
| | TGGAAGGTCG ATAATGCCCT GCAGAGTGGC | |
| | AATAGTCAGG AGTCCGTAAC CGAGCAGGAC | |
| | TCTAAGGACT CCACCTATTC CCTGAGTTCC | |
| | ACCTTGACCC TTTCCAAGGC CGACTATGAG | |
| | AAGCACAAAG TATACGCCTG CGAGGTAACT | |
| | CACCAGGGAT TGAGCTCCCC AGTGACAAAG | |
| | TCATTTAATC GGGGCGAGTG CCTGTCCAAG | |
| | GCCGACTACG AAAAGCACAA AGTGTACGCC | |
| | TGTGAAGTCA CCCATCAGGG CCTGTCATCT | |
| | CCAGTCACGA AGTCATTCAA TCGAGGGGAG | |
| | TGCCGGGCAA AACGGGCTCC CGTTAAACAG | |
| | ACGCTGAATT TCGATCTCCT GAAGTTGGCC | |
| | GGAGACGTCG AATCAAACCC CGGCCCAGGA | |
| | TGGAGCTGTA TCATCCTCTT CTTGGTAGCA | |
| | ACAGCTACAG GTAAGGGGTT AACAGTAGCA | |
| | GGCTTGAGGT CTGGACATAT ATATGGGTGA | |
| | CAATGACATC CACTTTGCCT TTCTCTCCAC | |
| | AGgcgcgcac tccGAAGTGC AGCTTGTGGA | |
| | GTCTGGCGGT GGCCTCGTGC AGCCAGGCCG | |
| | GAGCCTGCGG CTGAGCTGTG CAGCCAGCGG | |
| | GTTCACCTTC GATGATTATG CTATGCACT | |
| | GGGTTCGCCA GGCCCCCGGA AAGGGCCTGG | |
| | AGTGGGTCTC AGCTATCACA TGGAATTCCG | |
| | GACACATCGA CTACGCCGAC AGCGTGGAGG | |
| | GGCGCTTTAC CATTTCAAGG GACAACGCTA | |
| | AAAACAGCCT GTACCTTCAG ATGAACTCCC | |
| | TGCGGGCGGA AGACACAGCG GTGTACTACT | |
| | GTGCCAAGGT GAGCTACCTG TCCACAGCAT | |
| | CCTCATTGGA CTATTGGGGC CAAGGCACGC | |
| | TGGTTACCGT TTCCAGCGCA AGCACAAAGG | |
| | GACCTAGTGT GTTCCCGTTG GCCCCTTCAA | |
| | GCAAATCCAC GAGTGGAGGC ACCGCTGCAC | |
| | TGGGCTGCCT TGTAAAGGAC TACTTCCCGG | |
| | AGCCAGTGAC TGTGTCATGG AACAGTGGCG | |
| | CCCTGACAAG CGGAGTCCAC ACTTTTCCTG | |
| | CGGTCCTCCA GTCCTCCGGG CTTTACAGCC | |
| | TGAGTAGTGT GGTTACCGTC CCCTCATCCT | |
| | CCCTGGGTAC CCAGACCTAC ATTTGTAATG | |
| | TGAACCATAA GCCAAGCAAT ACAAAGGTGG | |
| | ATAAAAAGGT GGAGCCAAAA AGCTGCGATA | |
| | AAACACATAC TTGCCCTCCT TGCCCAGCGC | |
| | CCGAGTTGCT CGGCGGCCCT TCCGTATTTC | |
| | TTTTTCCACC GAAACCGAAG GATACACTGA | |
| | TGATCTCTCG GACCCCTGAG GTCACTTGTG | |
| | TGGTGGTTGA CGTTTCACAC GAGGACCCAG | |
| | AAGTGAAGTT TAATTGGTAC GTGGATGGGG | |
| | TTGAGGTGCA CAATGCTAAA ACCAAGCCGC | |
| | GCGAGGAGCA ATATAACTCT ACCTATCGAG | |
| | TGGTGAGCGT GCTCACCGTA CTCCATCAGG | |
| | ACTGGCTGAA CGGGAAGGAG TACAAGTGCA | |
| | AGGTTTCAAA CAAGGCTCTC CCTGCCCCAA | |
| | TAGAGAAGAC CATAAGTAAA GCCAAGGGAC | |
| | AGCCTCGCGA GCCACAGGTC TATACTCTGC | |
| | CTCCTAGTAG GGACGAGCTC ACCAAGAACC | |
| | AGGTAAGCCT CACCTGCTTG GTCAAGGGCT | |
| | TTTATCCATC CGACATCGCC GTGGAATGGG | |
| | AGAGCAACGG ACAGCCTGAA AACAACTACA | |
| | AAACTACCCC ACCCGTTCTT GATTCAGATG | |
| | GGAGCTTTTT TCTGTACAGC AAGTTGACCG | |
| | TCGATAAATC CCGATGGCAG CAGGGAAATG | |
| | TTTTCTCTTG CTCAGTGATG CATGAAGCGC | |
| | TGCACAACCA CTATACACAG AAGAGCCTTA | |
| | 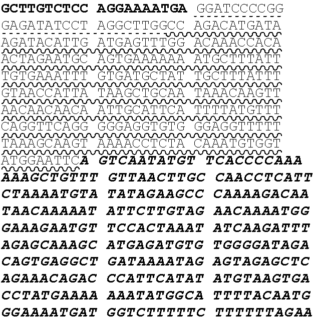 | |
| | 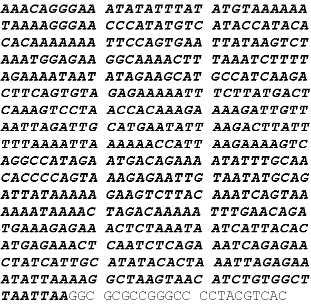 | |
| | CCGCCCCGTT CCCACGCCCC GCGCCACGTC | |
| | ACAAACTCCA CCCCCTCATT ATCATATTGG | |
| | CTTCAATCCA AAATAAGGTA TATTATTGAT | |
| | GATGGCCGCA GCGGCCCTGG CGTAATAGCG | |
| | AAGAGGCCCG CACCGATCGC CCTTCCCAAC | |
| | AGTTGCGCAG CCTGAATGGC GAATGGGACG | |
| | CGCCCTGTAG CGGCGCATTA AGCGCGGCGG | |
| | GTGTGGTGGT TACGCGCAGC GTGACCGCTA | |
| | CACTTGCCAG CGCCCTAGCG CCCGCTCCTT | |
| | TCGCTTTCTT CCCTTCCTTT CTCGCCACGT | |
| | TCGCCGGCTT TCCCCGTCAA GCTCTAAATC | |
| | GGGGGCTCCC TTTAGGGTTC CGATTTAGTG | |
| | CTTTACGGCA CCTCGACCCC AAAAAACTTG | |
| | ATTAGGGTGA TGGTTCACGT AGTGGGCCAT | |
| | CGCCCTGATA GACGGTTTTT CGCCCTTTGA | |
| | CGTTGGAGTC CACGTTCTTT AATAGTGGAC | |
| | TCTTGTTCCA AACTGGAACA ACACTCAACC | |
| | CTATCTCGGT CTATTCTTTT GATTTATAAG | |
| | GGATTTTGCC GATTTCGGCC TATTGGTTAA | |
| | AAAATGAGCT GATTTAACAA AAATTTAACG | |
| | CGAATTTTAA CAAAATATTA ACGCTTACAA | |
| | TTTAGGTGGC ACTTTTCGGG GAAATGTGCG | |
| | CGGAACCCCT ATTTGTTTAT TTTTCTAAAT | |
| | ACATTCAAAT ATGTATCCGC TCATGAGACA | |
| | ATAACCCTGA TAAATGCTTC AATAATATTG | |
| | AAAAAGGAAG AGTATGAGTA TTCAACATTT | |
| | CCGTGTCGCC CTTATTCCCT TTTTTGCGGC | |
| | ATTTTGCCTT CCTGTTTTTG CTCACCCAGA | |
| | AACGCTGGTG AAAGTAAAAG ATGCTGAAGA | |
| | TCAGTTGGGT GCACGAGTGG GTTACATCGA | |
| | ACTGGATCTC AACAGCGGTA AGATCCTTGA | |
| | GAGTTTTCGC CCCGAAGAAC GTTTTCCAAT | |
| | GATGAGCACT TTTAAAGTTC TGCTATGTGG | |
| | CGCGGTATTA TCCCGTATTG ACGCCGGGCA | |
| | AGAGCAACTC GGTCGCCGCA TACACTATTC | |
| | TCAGAATGAC TTGGTTGAGT ACTCACCAGT | |
| | CACAGAAAAG CATCTTACGG ATGGCATGAC | |
| | AGTAAGAGAA TTATGCAGTG CTGCCATAAC | |
| | CATGAGTGAT AACACTGCGG CCAACTTACT | |
| | TCTGACAACG ATCGGAGGAC CGAAGGAGCT | |
| | AACCGCTTTT TTGCACAACA TGGGGGATCA | |
| | TGTAACTCGC CTTGATCGTT GGGAACCGGA | |
| | GCTGAATGAA GCCATACCAA ACGACGAGCG | |
| | TGACACCACG ATGCCTGTAG CAATGGCAAC | |
| | AACGTTGCGC AAACTATTAA CTGGCGAACT | |
| | ACTTACTCTA GCTTCCCGGC AACAATTAAT | |
| | AGACTGGATG GAGGCGGATA AAGTTGCAGG | |
| | ACCACTTCTG CGCTCGGCCC TTCCGGCTGG | |
| | CTGGTTTATT GCTGATAAAT CTGGAGCCGG | |
| | TGAGCGTGGG TCTCGCGGTA TCATTGCAGC | |
| | ACTGGGGCCA GATGGTAAGC CCTCCCGTAT | |
| | CGTAGTTATC TACACGACGG GGAGTCAGGC | |
| | AACTATGGAT GAACGAAATA GACAGATCGC | |
| | TGAGATAGGT GCCTCACTGA TTAAGCATTG | |
| | GTAACTGTCA GACCAAGTTT ACTCATATAT | |
| | ACTTTAGATT GATTTAAAAC TTCATTTTTA | |
| | ATTTAAAAGG ATCTAGGTGA AGATCCTTTT | |
| | TGATAATCTC ATGACCAAAA TCCCTTAACG | |
| | TGAGTTTTCG TTCCACTGAG CGTCAGACCC | |
| | CGTAGAAAAG ATCAAAGGAT CTTCTTGAGA | |
| | TCCTTTTTTT CTGCGCGTAA TCTGCTGCTT | |
| | GCAAACAAAA AAACCACCGC TACCAGCGGT | |
| | GGTTTGTTTG CCGGATCAAG AGCTACCAAC | |
| | TCTTTTTCCG AAGGTAACTG GCTTCAGCAG | |
| | AGCGCAGATA CCAAATACTG TTCTTCTAGT | |
| | GTAGCCGTAG TTAGGCCACC ACTTCAAGAA | |
| | CTCTGTAGCA CCGCCTACAT ACCTCGCTCT | |
| | GCTAATCCTG TTACCAGTGG CTGCTGCCAG | |
| | TGGCGATAAG TCGTGTCTTA CCGGGTTGGA | |
| | CTCAAGACGA TAGTTACCGG ATAAGGCGCA | |
| | GCGGTCGGGC TGAACGGGGG GTTCGTGCAC | |
| | ACAGCCCAGC TTGGAGCGAA CGACCTACAC | |
| | CGAACTGAGA TACCTACAGC GTGAGCTATG | |
| | AGAAAGCGCC ACGCTTCCCG AAGGGAGAAA | |
| | GGCGGACAGG TATCCGGTAA GCGGCAGGGT | |
| | CGGAACAGGA GAGCGCACGA GGGAGCTTCC | |
| | AGGGGGAAAC GCCTGGTATC TTTATAGTCC | |
| | TGTCGGGTTT CGCCACCTCT GACTTGAGCG | |
| | TCGATTTTTG TGATGCTCGT CAGGGGGGCG | |
| | GAGCCTATGG AAAAACGCCA GCAACGCGGC | |
| | CTTTTTACGG TTCCTGGCCT TTTGCTGGCC | |
| | TTTTGCTCAC ATGTTCTTTC CTGCGTTATC | |
| | CCCTGATTCT GTGGATAACC GTATTACCGC | |
| | CTTTGAGTGA GCTGATACCG CTCGCCGCAG | |
| | CCGAACGACC GAGCGCAGCG AGTCAGTGAG | |
| | CGAGGAAGCG GAAGAGCGCC CAATACGCAA | |
| | ACCGCCTCTC CCCGCGCGTT GGCCGATTCA | |
| | TTAATGCAGG GGCCGCTGCG GCCATCATCA | |
| | ATAATATACC TTATTTTGGA TTGAAGCCAA TA | |
| | 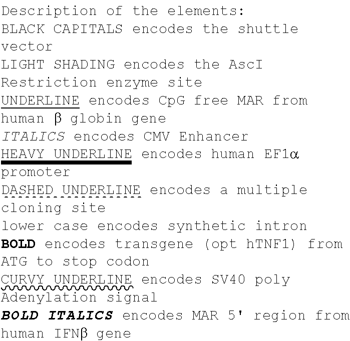 | |
| Sequence of | GGCCGATTCA TTAATGCAGG GGCCGCTGCG | 17 |
| pAd-MAR- | GCCATCATCA ATAATATACC TTATTTTGGA | |
| EF1a-opt | TTGAAGCCAA TATGATAATG AGGGGGTGGA | |
| hTNF3 | GTTTGTGACG TGGCGCGGGG CGTGGGAACG | |
| | GGGCGGGTGA CGTAGTAGTG TGGCGGAAGT | |
| | GTGATGTTGC AAGTGTGGCG GAACACATGT | |
| | AAGCGACGGA TGTGGCAAAA GTGACGTTTT | |
| | TGGTGTGCGC CGGTGTACAC AGGAAGTGAC | |
| | AATTTTCGCG CGGTTTTAGG CGGATGATTT | |
| | AGTAAATTTG GGCGTAACCG AGTAAGATTT | |
| | GGCCATTTTC GCGGGAAAAC TGAATAAGAG | |
| | GAAGTGAAAT CTGAATAATT TTGTGTTACT | |
| | CATAGCGCGT AATATTTGTC TAGGGCCGCG | |
| | GGGACTTTGA CCGTTTACGT GGAGACTCGC | |
| | CCAGGTGTTT TTCTCAGGTG TTTTCCGCGT | |
| | TCCGGGTCAA AGTTGGCGTT TTATTATTAT | |
| | AGTCAGCTGA CGTGTAGTGT ATTTATACCC | |
| | GGTGAGTTCC TCAAGAGGCC ACTCTTGAGT | |
| | GCCAGCGAGT AGAGTTTTCT CCTCCGAGCC | |
| | GCTCCGACAC CGGGAGGCGC GCCTTAATTA | |
| | AAATTATCTC TAAGGCATGT GAACTGGCTG | |
| | TCTTGGTTTT CATCTGTACT TCATCTGCTA | |
| | CCTCTGTGAC CTGAAACATA TTTATAATTC | |
| | CATTAAGCTG TGCATATGAT AGATTTATCA | |
| | TATGTATTTT CCTTAAAGGA TTTTTGTAAG | |
| | AACTAATTGA ATTGATACCT GTAAAGTCTT | |
| | TATCACACTA CCCAATAAAT AATAAATCTC | |
| | TTTGTTCAGC TCTCTGTTTC TATAAATATG | |
| | TACCAGTTTT ATTGTTTTTA GTGGTAGTGA | |
| | TTTTATTCTC TTTCTATATA TATACACACA | |
| | CATGTGTGCA TTCATAAATA TATACAATTT | |
| | TTATGAATAA AAAATTATTA GCAATCAATA | |
| | TTGAAAACCA CTGATTTTTG TTTATGTGAG | |
| | CAAACAGCAG ATTAAAAGGC TAGCCTGCAG | |
| | GAGTCAATGG GAAAAACCCA TTGGAGCCAA | |
| | GTACACTGAC TCAATAGGGA CTTTCCATTG | |
| | GGTTTTGCCC AGTACATAAG GTCAATAGGG | |
| | GGTGAGTCAA CAGGAAAGTC CCATTGGAGC | |
| | CAAGTACATT GAGTCAATAG GGACTTTCCA | |
| | ATGGGTTTTG CCCAGTACAT AAGGTCAATG | |
| | GGAGGTAAGC CAATGGGTTT TTCCCATTAC | |
| | TGACATGTAT ACTGAGTCAT TAGGGACTTT | |
| | CCAATGGGTT TTGCCCAGTA CATAAGGTCA | |
| | ATAGGGGTGA ATCAACAGGA AAGTCCCATT | |
| | GGAGCCAAGT ACACTGAGTC AATAGGGACT | |
| | TTCCATTGGG TTTTGCCCAG TACAAAAGGT | |
| | CAATAGGGGG TGAGTCAATG GGTTTTTCCC | |
| | ATTATTGGCA CATACATAAG GTCAATAGGG | |
| | 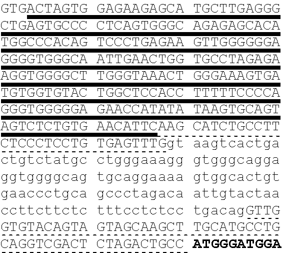 | |
| | GCTGTATCAT CCTCTTCTTG GTAGCAACAG | |
| | CTACAGGTAA GGGGTTAACA GTAGCAGGCT | |
| | TGAGGTCTGG ACATATATAT GGGTGACAAT | |
| | GACATCCACT TTGCCTTTCT CTCCACAGgc | |
| | gcgcactccG ACATCCAGAT GACGCAGTCC | |
| | CCAAGCTCAC TGTCCGCCTC TGTAGGTGAC | |
| | CGGGTAACTA TCACCTGCAG AGCATCCCAG | |
| | GGCATCCGCA ATTACCTGGC CTGGTATCAG | |
| | CAGAAACCTG GCAAGGCCCC AAAACTCCTC | |
| | ATCTACGCAG CATCCACCCT TCAGAGTGGC | |
| | GTACCAAGCC GATTCTCCGG AAGCGGTAGT | |
| | GGAACCGACT TTACCCTCAC AATCTCAAGT | |
| | CTGCAGCCTG AAGATGTCGC TACATATTAT | |
| | TGCCAGAGAT ACAATAGGGC CCCATACACC | |
| | TTTGGGCAGG GCACGAAAGT GGAAATTAAG | |
| | CGCACAGTTG CGGCACCAAG TGTGTTTATT | |
| | TTCCCGCCCA GCGATGAACA GCTGAAATCC CGGCCA | |
| | GCGTTGTATG CTTGCTGAAT AACTTTTACC | |
| | CTAGAGAGGC CAAGG | |
| | TCCAATGGAA GGTTGACAAC GCACTGCAGT | |
| | CCGGCAACAG TCAAGAGAGC GTCACTGAAC | |
| | AAGATTCCAA GGACAGTACA TACTCACTCA | |
| | GCTCCACACT GACACTCTCC AAGGCCGACT | |
| | ACGAGAAGCA TAAGGTCTAC GCTTGCGAGG | |
| | TAACGCATCA GGGCCTTTCT AGCCCAGTTA | |
| | CCAAAAGTTT CAATCGAGGC GAATGCCTGT | |
| | CAAAAGCAGA CTACGAGAAA CACAAGGTTT | |
| | ACGCCTGTGA AGTGACACAC CAGGGCTTGA | |
| | GCTCCCCTGT GACAAAATCT TTTAATAGGG | |
| | GAGAGTGTtg aATGATAATA TGGCCACAAC | |
| | CATGATGGGA TGGAGCTGTA TCATCCTCTT | |
| | CTTGGTAGCA ACAGCTACAG GTAAGGGGTT | |
| | AACAGTAGCA GGCTTGAGGT CTGGACATAT | |
| | ATATGGGTGA CAATGACATC CACTTTGCCT | |
| | TTCTCTCCAC AGgcgcgcac tccGAAGTGC | |
| | AGTTGGTCGA GTCCGGTGGA GGGCTGGTCC | |
| | AGCCTGGCAG AAGTCTCCGG CTGAGTTGCG | |
| | CAGCCAGCGG ATTCACCTTC GACGATTACG | |
| | CCATGCACTG GGTGCGGCAG GCCCCGGGCA | |
| | AGGGCCTTGA ATGGGTGTCT GCGATCACAT | |
| | GGAATTCCGG ACATATTGAT TACGCCGACA | |
| | GCGTGGAGGG CCGATTCACC ATCAGTAGGG | |
| | ATAATGCTAA GAACTCCCTG TACCTGCAGA | |
| | TGAATAGTCT GAGGGCTGAA GACACAGCCG | |
| | TGTACTATTG CGCAAAAGTC AGCTACCTCT | |
| | CCACTGCTTC TAGTCTGGAC TACTGGGGTC | |
| | AGGGGACGCT GGTGACGGTT TCTTCCGCAT | |
| | CCACTAAAGG TCCTAGCGTT TTCCCCCTCG | |
| | CCCCCTCTTC TAAGAGCACC TCCGGAGGAA | |
| | CTGCAGCCCT TGGATGCTTG GTTAAAGATT | |
| | ACTTTCCCGA ACCCGTAACC GTAAGCTGGA | |
| | ACAGTGGCGC CCTGACTTCA GGGGTACACA | |
| | CCTTTCCGGC CGTGCTGCAG AGCAGCGGGC | |
| | TCTATAGCCT TAGCTCAGTC GTGACGGTCC | |
| | CATCCTCTAG TCTTGGTACT CAAACCTACA | |
| | TCTGCAATGT GAATCACAAG CCTTCTAACA | |
| | CAAAAGTTGA TAAGAAAGTA GAACCCAAGA | |
| | GCTGTGATAA GACACATACT TGTCCTCCCT | |
| | GTCCGGCCCC CGAATTGCTT GGGGGGCCGA | |
| | GTGTCTTCCT CTTCCCTCCA AAACCCAAGG | |
| | ACACTCTCAT GATTTCAAGG ACCCCTGAAG | |
| | TGACTTGTGT GGTAGTTGAC GTGAGCCACG | |
| | AGGACCCTGA AGTGAAGTTC AATTGGTATG | |
| | TGGATGGCGT TGAGGTGCAT AATGCAAAGA | |
| | CAAAGCCACG CGAGGAGCAG TACAATTCCA | |
| | CCTATAGGGT GGTATCCGTG CTGACCGTGT | |
| | TGCATCAGGA CTGGCTCAAT GGGAAAGAGT | |
| | ATAAATGTAA GGTGTCCAAT AAGGCCCTGC | |
| | CCGCTCCCAT TGAAAAAACA ATTTCAAAGG | |
| | CTAAGGGCCA ACCCCGCGAA CCACAAGTCT | |
| | ACACACTCCC CCCTAGTAGA GATGAGCTGA | |
| | CAAAAAATCA GGTGTCTCTC ACATGTCTGG | |
| | TAAAAGGCTT CTATCCTTCA GATATTGCTG | |
| | TGGAATGGGA ATCAAATGGG CAGCCAGAGA | |
| | ATAACTACAA AACGACACCC CCAGTCCTTG | |
| | ATAGTGACGG GTCCTTCTTC CTCTACTCTA | |
| | AACTCACCGT GGACAAGAGT AGATGGCAAC | |
| | AGGGCAATGT GTTCTCCTGT AGCGTCATGC | |
| | ATGAAGCACT GCACAATCAT TATACTCAGA | |
| | 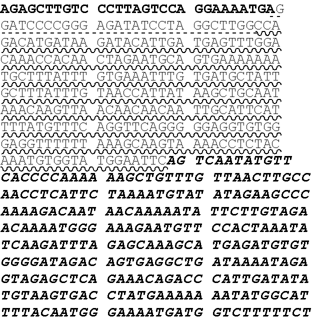 | |
| | 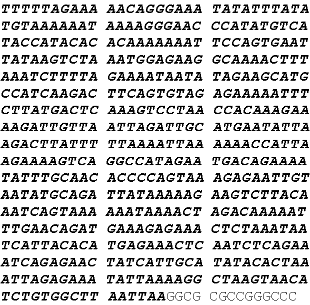 | |
| | CTACGTCACC CGCCCCGTTC CCACGCCCCG | |
| | CGCCACGTCA CAAACTCCAC CCCCTCATTA | |
| | TCATATTGGC TTCAATCCAA AATAAGGTAT | |
| | ATTATTGATG ATGGCCGCAG CGGCCCTGGC | |
| | GTAATAGCGA AGAGGCCCGC ACCGATCGCC | |
| | CTTCCCAACA GTTGCGCAGC CTGAATGGCG | |
| | AATGGGACGC GCCCTGTAGC GGCGCATTAA | |
| | GCGCGGCGGG TGTGGTGGTT ACGCGCAGCG | |
| | TGACCGCTAC ACTTGCCAGC GCCCTAGCGC | |
| | CCGCTCCTTT CGCTTTCTTC CCTTCCTTTC | |
| | TCGCCACGTT CGCCGGCTTT CCCCGTCAAG | |
| | CTCTAAATCG GGGGCTCCCT TTAGGGTTCC | |
| | GATTTAGTGC TTTACGGCAC CTCGACCCCA | |
| | AAAAACTTGA TTAGGGTGAT GGTTCACGTA | |
| | GTGGGCCATC GCCCTGATAG ACGGTTTTTC | |
| | GCCCTTTGAC GTTGGAGTCC ACGTTCTTTA | |
| | ATAGTGGACT CTTGTTCCAA ACTGGAACAA | |
| | CACTCAACCC TATCTCGGTC TATTCTTTTG | |
| | ATTTATAAGG GATTTTGCCG ATTTCGGCCT | |
| | ATTGGTTAAA AAATGAGCTG ATTTAACAAA | |
| | AATTTAACGC GAATTTTAAC AAAATATTAA | |
| | CGCTTACAAT TTAGGTGGCA CTTTTCGGGG | |
| | AAATGTGCGC GGAACCCCTA TTTGTTTATT | |
| | TTTCTAAATA CATTCAAATA TGTATCCGCT | |
| | CATGAGACAA TAACCCTGAT AAATGCTTCA | |
| | ATAATATTGA AAAAGGAAGA GTATGAGTAT | |
| | TCAACATTTC CGTGTCGCCC TTATTCCCTT | |
| | TTTTGCGGCA TTTTGCCTTC CTGTTTTTGC | |
| | TCACCCAGAA ACGCTGGTGA AAGTAAAAGA | |
| | TGCTGAAGAT CAGTTGGGTG CACGAGTGGG | |
| | TTACATCGAA CTGGATCTCA ACAGCGGTAA | |
| | GATCCTTGAG AGTTTTCGCC CCGAAGAACG | |
| | TTTTCCAATG ATGAGCACTT TTAAAGTTCT | |
| | GCTATGTGGC GCGGTATTAT CCCGTATTGA | |
| | CGCCGGGCAA GAGCAACTCG GTCGCCGCAT | |
| | ACACTATTCT CAGAATGACT TGGTTGAGTA | |
| | CTCACCAGTC ACAGAAAAGC ATCTTACGGA | |
| | TGGCATGACA GTAAGAGAAT TATGCAGTGC | |
| | TGCCATAACC ATGAGTGATA ACACTGCGGC | |
| | CAACTTACTT CTGACAACGA TCGGAGGACC | |
| | GAAGGAGCTA ACCGCTTTTT TGCACAACAT | |
| | GGGGGATCAT GTAACTCGCC TTGATCGTTG | |
| | GGAACCGGAG CTGAATGAAG CCATACCAAA | |
| | CGACGAGCGT GACACCACGA TGCCTGTAGC | |
| | AATGGCAACA ACGTTGCGCA AACTATTAAC | |
| | TGGCGAACTA CTTACTCTAG CTTCCCGGCA | |
| | ACAATTAATA GACTGGATGG AGGCGGATAA | |
| | AGTTGCAGGA CCACTTCTGC GCTCGGCCCT | |
| | TCCGGCTGGC TGGTTTATTG CTGATAAATC | |
| | TGGAGCCGGT GAGCGTGGGT CTCGCGGTAT | |
| | CATTGCAGCA CTGGGGCCAG ATGGTAAGCC | |
| | CTCCCGTATC GTAGTTATCT ACACGACGGG | |
| | GAGTCAGGCA ACTATGGATG AACGAAATAG | |
| | ACAGATCGCT GAGATAGGTG CCTCACTGAT | |
| | TAAGCATTGG TAACTGTCAG ACCAAGTTTA | |
| | CTCATATATA CTTTAGATTG ATTTAAAACT | |
| | TCATTTTTAA TTTAAAAGGA TCTAGGTGAA | |
| | GATCCTTTTT GATAATCTCA TGACCAAAAT | |
| | CCCTTAACGT GAGTTTTCGT TCCACTGAGC | |
| | GTCAGACCCC GTAGAAAAGA TCAAAGGATC | |
| | TTCTTGAGAT CCTTTTTTTC TGCGCGTAAT | |
| | CTGCTGCTTG CAAACAAAAA AACCACCGCT | |
| | ACCAGCGGTG GTTTGTTTGC CGGATCAAGA | |
| | GCTACCAACT CTTTTTCCGA AGGTAACTGG | |
| | CTTCAGCAGA GCGCAGATAC CAAATACTGT | |
| | TCTTCTAGTG TAGCCGTAGT TAGGCCACCA | |
| | CTTCAAGAAC TCTGTAGCAC CGCCTACATA | |
| | CCTCGCTCTG CTAATCCTGT TACCAGTGGC | |
| | TGCTGCCAGT | |
| | GGCGATAAGT CGTGTCTTAC CGGGTTGGAC | |
| | TCAAGACGAT AGTTACCGGA TAAGGCGCAG | |
| | CGGTCGGGCT GAACGGGGGG TTCGTGCACA | |
| | CAGCCCAGCT TGGAGCGAAC GACCTACACC | |
| | GAACTGAGAT ACCTACAGCG TGAGCTATGA | |
| | GAAAGCGCCA CGCTTCCCGA AGGGAGAAAG | |
| | GCGGACAGGT ATCCGGTAAG CGGCAGGGTC | |
| | GGAACAGGAG AGCGCACGAG GGAGCTTCCA | |
| | GGGGGAAACG CCTGGTATCT TTATAGTCCT | |
| | GTCGGGTTTC GCCACCTCTG ACTTGAGCGT | |
| | CGATTTTTGT GATGCTCGTC AGGGGGGCGG | |
| | AGCCTATGGA AAAACGCCAG CAACGCGGCC | |
| | TTTTTACGGT TCCTGGCCTT TTGCTGGCCT | |
| | TTTGCTCACA TGTTCTTTCC TGCGTTATCC | |
| | CCTGATTCTG TGGATAACCG TATTACCGCC | |
| | TTTGAGTGAG CTGATACCGC TCGCCGCAGC | |
| | CGAACGACCG AGCGCAGCGA GTCAGTGAGC | |
| | GAGGAAGCGG AAGAGCGCCC AATACGCAAA | |
| | CCGCCTCTCC CCGCGCGTTG GCCGATTCAT | |
| | TAATGCAGGG GCCGCTGCGG CCATCATCAA | |
| | TAATATACCT TATTTTGGAT TGAAGCCAAT A | |
| | 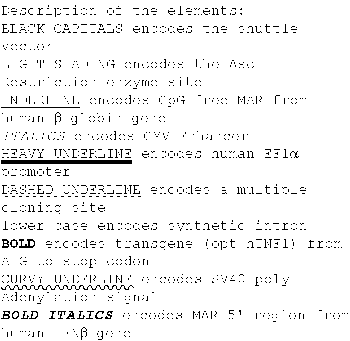 | |
| HDΔ28E4- | CATCATCAAT AATATACCTT ATTTTGGATT | 18 |
| MAR-EF1a- | GAAGCCAATA TGATAATGAG GGGGTGGAGT | |
| optHuman | TTGTGACGTG GCGCGGGGCG TGGGAACGGG | |
| EPO-1 | GCGGGTGACG TAGTAGTGTG GCGGAAGTGT | |
| | GATGTTGCAA GTGTGGCGGA ACACATGTAA | |
| | GCGACGGATG TGGCAAAAGT GACGTTTTTG | |
| | GTGTGCGCCG GTGTACACAG GAAGTGACAA | |
| | TTTTCGCGCG GTTTTAGGCG GATGTTGTAG | |
| | TAAATTTGGG CGTAACCGAG TAAGATTTGG | |
| | CCATTTTCGC GGGAAAACTG AATAAGAGGA | |
| | AGTGAAATCT GAATAATTTT GTGTTACTCA | |
| | TAGCGCGTAA TATTTGTCTA GGGCCGCGGG | |
| | GACTTTGACC GTTTACGTGG AGACTCGCCC | |
| | AGGTGTTTTT CTCAGGTGTT TTCCGCGTTC | |
| | CGGGTCAAAG TTGGCGTTTT GATATCAAGC | |
| | TTATCGATAC CGTAAACAAG TCTTTAATTC | |
| | AAGCAAGACT TTAACAAGTT AAAAGGAGCT | |
| | TATGGGTAGG AAGTAGTGTT ATGATGTATG | |
| | GGCATAAAGG GTTTTAATGG GATAGTGAAA | |
| | ATGTCTATAA TAATACTTAA ATGGCTGCCC | |
| | AATCACCTAC AGGATTGATG TAAACATGGA | |
| | AAAGGTCAAA AACTTGGGTC ACTAAAATAG | |
| | ATGATTAATG GAGAGGATGA GGTTGATAGT | |
| | TAAATGTAGA TAAGTGGTCT TATTCTCAAT | |
| | AAAAATGTGA ACATAAGGCG AGTTTCTACA | |
| | AAGATGGACA GGACTCATTC ATGAAACAGC | |
| | AAAAACTGGA CATTTGTTCT AATCTTTGAA | |
| | GAGTATGAAA AATTCCTATT TTAAAGGTAA | |
| | AACAGTAACT CACAGGAAAT ACCAACCCAA | |
| | CATAAAATCA GAAACAATAG TCTAAAGTAA | |
| | TAAAAATCAA ACGTTTGCAC GATCAAATTA | |
| | TGAATGAAAT TCACTACTAA AATTCACACT | |
| | GATTTTGTTT CATCCACAGT GTCAATGTTG | |
| | TGATGCATTT CAATTGTGTG ACACAGGCAG | |
| | ACTGTGGATC AAAAGTGGTT TCTGGTGCGA | |
| | CTTACTCTCT TGAGTATACC TGCAGTCCCC | |
| | TTTCTTAAGT GTGTTAAAAA AAAAGGGGGA | |
| | TTTCTTCAAT TCGCCAATAC TCTAGCTCTC | |
| | CATGTGCTTT CTAGGAAACA AGTGTTAACC | |
| | CACCTTATTT GTCAAACCTA GCTCCAAAGG | |
| | ACTTTTGACT CCCCACAAAC CGATGTAGCT | |
| | CAAGAGAGGG TATCTGTCAC CAGTATGTAT | |
| | AGTGAAAAAA GTATCCCAAG TCCCAACAGC | |
| | AATTCCTAAA AGGAGTTTAT TTAAAAAACC | |
| | ACACACACCT GTAAAATAAG TATATATCCT | |
| | CCAAGGTGAC TAGTTTTAAA AAAACAGTAT | |
| | TGGCTTTGAT GTAAAGTACT AGTGAATATG | |
| | TTAGAAAAAT CTCACTGTAA CCAAGTGAAA | |
| | TGAAAGCAAG TATGGTTTGC AGAGATTCAA | |
| | AGAAAATATA AGAAAACCTA CTGTTGCCAC | |
| | TAAAAAGAAT CATATATTAA ATATACTCAC | |
| | ACAATAGCTC TTCAGTCTGA TAAAATCTAC | |
| | AGTCATAGGA ATGGATCTAT CACTATTTCT | |
| | ATTCAGTGCT TTGATGTAAT CCAGCAGGTC | |
| | AGCAAAGAAT TTATAGCCCC CCTTGAGCAC | |
| | ACAGAGGGCT ACAATGTGAT GGCCTCCCAT | |
| | CTCCTTCATC ACATCTCGAG CAAGACGTTC | |
| | AGTCCTACAG AAATAAAATC AGGAATTTAA | |
| | TAGAAAGTTT CATACATTAA ACTTTATAAC | |
| | AAACACCTCT TAGTCATTAA ACTTCCACAC | |
| | CAACCTGGGC AATATAGTGA GACCCCATGC | |
| | CTGCAAAAAA AAAAAAATTA GCCAGGCATG | |
| | GTAGCATGTA CCTGTAGTCC CAGCTACTTG | |
| | AGAGGTGAGG TGGGAAAATC ACTTTAGTGC | |
| | AGGATGTTGA GGCTGGAGTG AACTGTGATT | |
| | GTGCCACTGC ACTCCAGCCT GGACAATAGA | |
| | GCAAGACCTT GTCTCAAAAA AATGCATTAA | |
| | AAATTTTTTT TAAATCTTCC ACGTATCACA | |
| | TCCTTTGCCC TCATGTTTCA TAAGGTAAAA | |
| | AATTTGATAC CTTCAAAAAA ACCAAGCATA | |
| | CCACTATCAT AATTTTTTTT AAATGCAAAT | |
| | AAAAACAAGA TACCATTTTC ACCTATCAGA | |
| | CTGGCAGGTT CTGATTAAAT GAAATTTTCT | |
| | GGATAATATA CAATATTAAG AGAGACTGTA | |
| | GAAACTGGGC CAGTGGCTCA TGCCTGTAAT | |
| | CCCAGCACTT TGGGAGGCTG GGTAACATGG | |
| | CGAACCCTGT TTCTACAAAA TAAAAATATT | |
| | AGCTGGGAGT GGTGGCGCAC ACCTATAGTC | |
| | CCAGCTACTC AGGAGGCTGA GGTGGAAGGA | |
| | TCGCTTGAAC CCAGGAGGTT GAGACTGCAG | |
| | TGAACTGTGA TCATTCTGCT GCACTGCACC | |
| | CCAGCCTGGG CAACAGAGAC CTTGTCTCAA | |
| | AAAAAAAAAA AAAAGAGACA AATTGTGAAG | |
| | AGAAAGGTAC TCTCATATAA CATCAGGAGT | |
| | ATAAAATGAT TCAACTTCTT AGAGGAAAAT | |
| | TTGGCAATAC CAAAATATTC AATAAACTCT | |
| | TTCCCCTTGA CCCAGAAATT CCACTTGAAT | |
| | AAAGCTGAAC AAGTACCAAA CATGTAAAAG | |
| | AATGTTTCTT CTAGTACAGT CGGTAAGAAC | |
| | AAAATAGTGT CTATCAATAG TGGACTGGTT | |
| | AAATCAGTTA TGGTATCTCC ATAAGACAGA | |
| | ATGCTATGCA ACCTTTAAAA TATATTAGAT | |
| | AGCTCTAGAC ACACTAATAT TAAAAGTGTC | |
| | CAATAACATT TAAAACTATA CTCATACGTT | |
| | AAAATATAAA TGTATATATG TACTTTTGCA | |
| | TATAGTATAC ATGCATAGGC CAGTGCTTGA | |
| | GAAGAAATGT GTACAGAAGG CTGAAAGGAG | |
| | AGAACTTTAG TCTTCTTGTT TATGGCCTCC | |
| | ATAGTTAGAA TATTTTATAA CACAAATATT | |
| | TTGATATTAT AATTTTAAAA TAAAAACACA | |
| | GAATAGCCAG ACATACAATG CAAGCATTCA | |
| | ATACCAGGTA AGGTTTTTCA CTGTAATTGA | |
| | CTTAACAGAA AATTTTCAAG CTAGATGTGC | |
| | ATAATAATAA AAATCTGACC TTGCCTTCAT | |
| | GTGATTCAGC CCCAGTCCAT TACCCTGTTT | |
| | AGGACTGAGA AATGCAAGAC TCTGGCTAGA | |
| | GTTCCTTCTT CCATCTCCCT TCAATGTTTA | |
| | CTTTGTTCTG GTCCCTACAG AGTCCCACTA | |
| | TACCACAACT GATACTAAGT AATTAGTAAG | |
| | GCCCTCCTCT TTTATTTTTA ATAAAGAAGA | |
| | TTTTAGAAAG CATCAGTTAT TTAATAAGTT | |
| | GGCCTAGTTT ATGTTCAAAT AGCAAGTACT | |
| | CAGAACAGCT GCTGATGTTT GAAATTAACA | |
| | CAAGAAAAAG TAAAAAACCT CATTTTAAGA | |
| | TCTTACTTAC CTGTCCATAA TTAGTCCATG | |
| | AGGAATAAAC ACCCTTTCCA AATCCTCAGC | |
| | ATAATGATTA GGTATGCAAA ATAAATCAAG | |
| | GTCATAACCT GGTTCATCAT CACTAATCTG | |
| | AAAAAGAAAT ATAGCTGTTT CAATGAGAGC | |
| | ATTACAGGAT ACAAACATTT GATTGGATTA | |
| | AGATGTTAAA AAATAACCTT AGTCTATCAG | |
| | AGAAATTTAG GTGTAAGATG ATATTAGTAA | |
| | CTGTTAACTT TGTAGGTATG ATAATGAATT | |
| | ATGTAAGAAA ACAACAGGCC GGGCGGGTTG | |
| | GTTCACACGT GTAATCCCAG CACTTTGGGA | |
| | GGCTGAGGCA GGCAGACTGC CTGAGCTCAG | |
| | GAGTTCGAGA CCAGCCTGGG CAACACGGTG | |
| | AAATCCCGTC TCTACTAAAA ATACAAAAAA | |
| | ATTAGCCGGG TGTGGTGACA CATGCCTGTA | |
| | GTCCCAGCTA CTTGGGAGGC TGAGGCAGGA | |
| | GAATCACTTG AACCTGGGAG GTGAAGGTTG | |
| | CAGTGAGCCA AGATGGCACC ACTTCACTCC | |
| | AGCCTGGGAA ACAGAGCAAG ACTCTGTCTC | |
| | TGAGCTGAGA TGGCACCACT TCACTCCAGC | |
| | CTGGGAAACA GAGCAAGACT CTGTCTCAAA | |
| | AAAAACAAAA CACACAAACA AAAAAACAGG | |
| | CTGGGCGCGG TGGCTCACGC CTGTAATCCC | |
| | AGCACTTTGG GAGGCCGAGG CGGGTGGATC | |
| | ACCTGAGGTC AGGAGTTCCA GACCAGCCTT | |
| | GTCAACATGG TGAAACCTCC CCCCGCCGTC | |
| | TCTACTAAAA ATACAAAAAT TAGCCAGGCG | |
| | TGGTGGCAGG AGCCTGTAAT CCCAGCTACT | |
| | TGGGAGGCTG AGGCAGGAGA ATCGCTTGTA | |
| | CCCAGAAGGC AGAGGTTGCA CTGAGCTGAG | |
| | ATGGCACCAT TGCACTCCAG CCTGGGGGAC | |
| | AAGAGCGAGA TTTCGTCTTT AAAAAACAAA | |
| | AACAAAACAA AAAACCATGT AACTATATGT | |
| | CTTAGTCATC TTAGTCAAGA ATGTAGAAGT | |
| | AAAGTGATAA GATATGGAAT TTCCTTTAGG | |
| | TCACAAAGAG AAAAAGAAAA ATTTTAAAGA | |
| | GCTAAGACAA ACGCAGCAAA ATCTTTATAT | |
| | TTAATAATAT TCTAAACATG GGTGATGAAC | |
| | ATACGGGTAT TCATTATACT ATTCTCTCCA | |
| | CTTTTGAGTA TGTTTGAAAA TTTAGTAAAA | |
| | CAAGTTTTAA CACACTGTAG TCTAACAAGA | |
| | TAAAATATCA CACTGAACAG GAAAAACTGG | |
| | CATGGTGTGG TGGCTCACAC TTGTAATCCC | |
| | AGTGCTTTGG GAGGCTGAGA CAGGAGAGTT | |
| | GCTTGAGGCC AGGAGTTCAA GACCGACATG | |
| | GGGAATGTAG CAAGACCCCG TCCCTACAAA | |
| | AAACTTTGTA AAAATTTGCC AGGTATGGTG | |
| | GTGCATACCT GTAGTCCCAG CTACTCGGGA | |
| | GGCGGAGGCA GAAGGAATCA CTTGAGCCCA | |
| | GGAGTTTGAG GCTGCAGTGA GCTACGATCA | |
| | TACCACAGCA CTCCAGCGTG GACAACAGAG | |
| | TAAGACCCTA TCTCAAAAAC AAAACAAAAC | |
| | AAAACAAACA AAAAAAACCA CAAGAAAAAC | |
| | TGCTGGCTGA TGCAGCGGCT CATGCCTGTA | |
| | ATCCCAGTAT TTTGGGAGGC CCAGGTGGGC | |
| | GTATCACCTG AGGTCAGGAG TTAGAGACCA | |
| | GCCTGGCCAA CATGGTGAAA CCCCATCTCT | |
| | ACTAAAAATA CAAAATTAGC CAGGCATGTG | |
| | GCACGCGCCT GTAGTCCCAG TTACTGGGAG | |
| | GCTGAAGCAG GAGGATCACC TGAGCCCGGG | |
| | AGGTGGAGGT TGCAGTGAGC CGAGATCACA | |
| | CCACTGCACT CCAGCCTGGG TGACACAGCA | |
| | ATACCCTACC TCAAAATAAA AAAGAAAAAG | |
| | AAAAGAAAAG TTGCTGTCCC CGCTACCCCA | |
| | ATCCCAAATC CAAACAGCCT CTCTCATCTC | |
| | ACAGTAAGGG GGAAAAATCA CCCAAAAAAG | |
| | CTAAGTGATC TTTTGAAAAC CCAAACTCTT | |
| | AGAAGTCTAA GATTATTATA GTCAACTCAT | |
| | GAAGTGTCAT CATAAAAGAT ACTCTAATAT | |
| | TATTTAAGTA GAACCACATA TTGGTTGTCT | |
| | TGGTATGTCT AGCCCCTGGC ATACAAAATA | |
| | TTTAATAACA CTGATATGGT ACCTGTGATG | |
| | TGAAAATGTA CTATGAGTAC AGCTTTATAA | |
| | ATACTATATA TGTACCTATA TACAGAAAAA | |
| | AATACAACAA AATCATAAAA GCACTTATCT | |
| | TTGAAAGAGG AGTTACAGCA ATTTTATTTA | |
| | GTTCTTTATT GCTTTGCTAT ATATTCTAAA | |
| | TTTTTTTCAA TGAATATATA TCACTTTTAA | |
| | AAAAATTCAA TGGTCTTTCT TATAAATTAT | |
| | CTTTGGCAGC ATGCGTTTTT ATATATACAT | |
| | ATAAAATGTA TGGGAAATTT TTAAAGGATA | |
| | CATTAAATTA AAGCAAAATA TACAAACAAA | |
| | AAATCAGAAT ACAAAAAGAT AAAAAGATTG | |
| | GGAAGGGAGG GAGGGAGTAA GGAGGAAGGG | |
| | TGGGTGGGTA TAGAGAAATA TACCAAATAA | |
| | TGGTAAGAAG TGGGGTCTTG ACACTTTCTA | |
| | CACTTTTTTT AAATAAAAAA AATTTTTTTC | |
| | TCTCTCTTTT TTTTTTTTAG AGACGAAGTC | |
| | TCGCTATGTT GCCCAGGCTG GTCTTGAACT | |
| | CCTGGGATCA AGAGATCCTC CTGCCTCAGC | |
| | CTCCCAAGGT GCTTGGATTA CAGGTGTGAG | |
| | CCACCACGCC TGGTCACTTT CTACACTTTA | |
| | ATATATATAT TTTTTCATTT TCAATGTCAT | |
| | TTTTATTAGT TAATTTATAA TACCCATTCA | |
| | CCATTATATT CAAAGTCTAT TTGAAGAAAT | |
| | AAACCAGAAA GAATGAAATA CTCTAGCTCA | |
| | CATGCTATTC AATACTAAAT TACCTTTCAA | |
| | ATCACATTCA AGAAGCTGAT GATTTAAGCT | |
| | TTGGCGGTTT CCAATAAATA TTGGTCAAAC | |
| | CATAATTAAA TCTCAATATA TCAGTTAGTA | |
| | CCTATTGAGC ATCTCCTTTT ACAACCTAAG | |
| | CATTGTATTA GGTGCTTAAA TACAAGCAGC | |
| | TTGACTTTTA ATACATTTAA AAATACATAT | |
| | TTAAGACTTA AAATCTTATT TATGGAATTC | |
| | AGTTATATTT TGAGGTTTCC AGTGCTGAGA | |
| | AATTTGAGGT TTGTGCTGTC TTTCAGTCCC | |
| | CAAAGCTCAG TTCTGAGTTC TCAGACTTTG | |
| | GTGGAACTTC ATGTATTGTC AGGTTGGCCC | |
| | GTAATACCTG TGGGACAACT TCAGCCCCTG | |
| | TGCACATGGC CAGGAGGCTG GTTGCAAACA | |
| | TTTTCAGGTA GGTGGACCAG GACATGCCCC | |
| | TGGTCATGGC CAGGTGGAGG CATAGTGCTA | |
| | TACAGCAGGC AGAAGTCAAT ATTGATTTGT | |
| | TTTTAAAGAA ACATGTACTA CTTTCATAAG | |
| | CAGAAAAAAT TTCTATTCTT GGGGGAAAAG | |
| | ATTATGCCAG ATCCTCTAGG ATTAAATGCT | |
| | GATGCATCTG CTAAACCTTC ACATATCAGA | |
| | ACATATTTAC TATAGAAAGA ATGAAAATGG | |
| | GACATTTGTG TGTCACCTAT GTGAACATTC | |
| | CAAAAATATT TTACAACAAC TAAGTATTTT | |
| | ATAAATTTTA TGAACTGAAA TTTAGTTCAA | |
| | GTTCTAGGAA AATACAAACC TTGCTAGATA | |
| | TTATAAAAAT GATACAATAT ATATTCATTT | |
| | CAGGCTCATC AGAATATATC TGTTATCACT | |
| | TGACAAGAAT GAAAATGCAC CATTTTGTAG | |
| | TGCTTTAAAA TCAGGAAGAT CCAGAGTACT | |
| | AAAAATGACT TCTTCCTTGA AGCTTACTCA | |
| | CCAACTTCCT CCCAGTTACT CACTGCTTCT | |
| | GCCACAAGCA TAAACTAGGA CCCAGCCAGA | |
| | ACTCCCTTGA AATATACACT TGCAACGATT | |
| | ACTGCATCTA TCAAAATGGT TCAGTGCCTG | |
| | GCTACAGGTT CTGCAGATCG ACTAAGAATT | |
| | TGAAAAGTCT TGTTTATTTC AAAGGAAGCC | |
| | CATGTGAATT CTGCCCAGAG TTCATCCCAG | |
| | ATATGCAGTC TAAGAATACA GACAGATCAG | |
| | CAGAGATGTA TTCTAAAACA GGAATTCTGG | |
| | CAATATAACA AATTGATTTC CAATCAAAAC | |
| | AGATTTACAT ACCATACTTA TGTCAAGAAG | |
| | TTGTTTTGTT TTATTGCATC CTAGATTTTA | |
| | TTTTTTTGAT TTATGGTTTA CTTTAAGCAT | |
| | AAAAAATTTG TCAATACAAC TCTTCCCAAA | |
| | AGGCATAAAC AAAAATTCAT AAAACTTGCA | |
| | TCACTTGAGA TACTTCAGGT ATGAATTCAC | |
| | AACTTTGTTA CAACTTACTA TATATATGCA | |
| | CACATATATA TATATTTGGG TATATTGGGG | |
| | GGGTTCTAAT TTAAGAAATG CATAATTGGC | |
| | TATAGACAGA CAGTTGTCAG AACTTGGCAA | |
| | TGGGTACGTG CAGGTTCATT ATACCAAGTC | |
| | TACTTGTAGT TGTTCAAAAT GTATCATAAT | |
| | ACAAGGCCGG GCGAGGTCGT CACGCCTGTA | |
| | ATCCCAGCAT TTTGGGAGGC TAAGGCAGGA | |
| | GGATTGCTTG AGGTCAGGAG TTTGTGACCA | |
| | GCCTGGGCAA CAGAGCAAGA CCCTGTCTCC | |
| | AAAAAGAAAA AAAATAATTT TTTACAAAAT | |
| | AAAAACAAAA TGTATCATCA GACGAAATTA | |
| | AATAAGAGGC AATTCATTTA AATGACAACT | |
| | TTTCCCAGCT TGACATTTAA CAAAAAGTCT | |
| | AAGTCCTCTT AATTCATATT TAATGATCAA | |
| | ATATCAAATA CTAATTTTTT TTTTTTTTTT | |
| | TTTTTTGAGA CGGAGTCTCG CTCTGTCGCC | |
| | CAGGCTGGAG TGCAGTGGCG CGATCCTGGC | |
| | TCACTGCAAG CTCCGCCTCC CGGGTTCACG | |
| | CCATTCTCCT GCCTCAGCCT CCCGAGTAGC | |
| | TGGGATTACA GACATGCGCC ACCACGCCCG | |
| | GCTAATTTTG TATTTTTAGT AGAGATGGGG | |
| | TTTCTCCATG TTGGTCAGGC TGGTCTTGAA | |
| | TTTCCCACCT CAGGTGATCT GCCTGCCTCA | |
| | GCCTCACAAA GCAGTAGCTG GGACTACAGG | |
| | CACCCACCAC CACACTTGGT TAATTCTTTT | |
| | GTATTTTTTT TGTAAAGACG GGATTTCACC | |
| | ATGTTAGCCA GGATGGTCTC GATCTCCTGA | |
| | TCTCATGATC CGCCCGCCTC AGCCTCCCAA | |
| | AGTGCTGGGA TTACAGGCGT GAGCCACCCC | |
| | GCCCGGCCAT CAAATACTAA TTCTTAAATG | |
| | GTAAGGACCC ACTATTCAGA ACCTGTATCC | |
| | TTATCACTAA TATGCAAATA TTTATTGAAT | |
| | ACTTACTATG TCATGCATAC TAGAGAGAGT | |
| | TAGATAAATT TGATACAGCT ACCCTCACAG | |
| | AACTTACAGT GTAATAGATG GCATGACATG | |
| | TACATGAGTA ACTGTGAACA GTGTTAAATT | |
| | GCTATTTAAA AAAAAAGACG GCTGGGCGCT | |
| | GTGGCTCATG CCTGTAATCC CAGCACTTTG | |
| | GGAGGCCAAG GCAAGTTGAT CGCTCGAGGT | |
| | CAAGAGTTCG AGACCAGCCT GGCCAACGTG | |
| | GTAAAACCCC GTCTCTACTA AAAATACAAA | |
| | AAAAAAATTA GCCAGGCATG GTGGCACAGG | |
| | CCTGTAATCC CAGCTACTAG GGAGGCTGAG | |
| | ACATGGAGAA CTGCTTGAAT CCAGGAGGCA | |
| | GAGGTTACAG TGAGCCGAGA TCATACCACT | |
| | ACACTCCAGC CTGAGTGACA GAGCGAGACT | |
| | CCTGTCTAAA AAAAAAAAAA AAAAAAAAGA | |
| | TACAGGTTAA GTGTTATGGT AGTTGAAGAG | |
| | AGAACTCAAA CTCTGTCTCA GAAGCCTCAC | |
| | TTGCATGTGG ACCACTGATA TGAAATAATA | |
| | TAAATAGGTA TAATTCAATA AATAGGAACT | |
| | TCAGTTTTAA TCATCCCAAA CACCAAAACT | |
| | TCCTATCAAA CAGGTCCAAT AAACTCAATC | |
| | TCTATAAGAG CTAGACAGAA ATCTACTTGG | |
| | TGGCCTATAA TCTTATTAGC CCTTACTTGT | |
| | CCCATCTGAT ATTAATTAAC CCCATCTAAT | |
| | ATGGATTAGT TAACAATCCA GTGGCTGCTT | |
| | TGACAGGAAC AGTTGGAGAG AGTTGGGGAT | |
| | TGCAACATAT TCAATTATAC AAAAATGCAT | |
| | TCAGCATCTA CCTTGATTAA GGCAGTGTGC | |
| | AACAGAATTT GCAGGAGAGT AAAAGAATGA | |
| | TTATAAATTT ACAACCCTTA AAGAGCTATA | |
| | GCTGGGCGTG GTGGCTCATG CCTGTAAATC | |
| | CCAGCACTTT GGGAGGCTGA GGCGGGTGGA | |
| | TCACCTGAGG CCAGAAGTTC AAGACCAGCC | |
| | TAGCCAACAT GGCGAAACCC TGTCTCTACA | |
| | AAAAATACAA AAATTAGCCG GGTGTGGTGG | |
| | CACGTGCCTG TAGTCCCAGT TACTTGGGAG | |
| | GCCGAGGCAG GAGAATCGCT TGAACCTAGG | |
| | AGGTGGAGGC TGCAGTGAGC CGAGATTGTG | |
| | CCACTGCACT CCACTTCAGC CTGGGCGACA | |
| | AGAGCAAGAC TCCGTCACAA AAAAAAALWI | |
| | AAAAAAAAPG CTTAAAATCT AGTGGGAAAG | |
| | GCATATATAC ATACAACTAA CTGTATAGCA | |
| | TAATAAAGCT CATAATCTGT AACAAAATCT | |
| | AATTCGACAA GCCCAGAAAC TTGTGATTTA | |
| | CCAAAAACAG TTATATATAC ACAAAAAGTA | |
| | AACCTAGAAC CCAAAGTTAC CCAGCACCAA | |
| | TGATTCTCTC CCTAAGCAGT ATCAAGTTTA | |
| | AAGCAGTGAT TACATTCTAC TGCCTAGATT | |
| | GTAAACTGAG TAAAGGAGAC CAGCACCTTT | |
| | CTGCTACTGA ACTAGCACAG CCGTGTAAAC | |
| | CAACAAGGCA ATGGCAGTGC CCAACTTTCT | |
| | GTATGAATAT AAGTTACATC TGTTTTATTA | |
| | TTTGTGACTT GGTGTTGCAT GTGGTTATTA | |
| | TCAACACCTT CTGAAAGAAC AACTACCTGC | |
| | TCAGGCTGCC ATAACAAAAT ACCACAGACT | |
| | GAGTGACTTA ACAGAAACTT ATTTCTCACA | |
| | GTTTTGGAGG CTGGGAAGTC CAAAATTAAG | |
| | GTACCTGCAA GGTAGGTTTC AATCTCAGGC | |
| | CTCTTCTTTG GCTTGAAGGT CTTCTAACTG | |
| | TGTGCTCACA TGACCTCTTC TAACAAGCTC | |
| | TCTGGTGTCT CTTTTTTTTT TTTTTTCTTT | |
| | TTTGAGACAG AGTCTCACTC TGTCACCCAG | |
| | GCTGGAGTAC AGTGGCACAA TCTGGGCTCA | |
| | CTGCAACCTC CAACTCCCGG GTTCAAGTGA | |
| | TTCTCATGCC TCACCCTCCC GAGTAGCTTG | |
| | GATGACAGGA GCCCGCTACC ACACCCAGCT | |
| | AATTTTTGTA TTTTTAGTAG AGATGGTGTT | |
| | TCACTACATT GGCCAGGCTG GTCTCAAACT | |
| | CCTGACCTCG TGATCCACCC ACCTTGGCCT | |
| | CCCAAAGTGC TGGGATTACA GGTGTGAGCC | |
| | ACTGCGCCCG TCCTGGTGTC TTTTCATATA | |
| | AGGGCACTAA TCCAATCAGA CCTGGGCCCA | |
| | ACCCTCCCGA CTTCTTCTAA CTGTAATTAC | |
| | CTTCCAAAGG CCCTGTCTCC AAATACCATC | |
| | ACACTGGGGG TTAGGACTTC AAAAAAGGTA | |
| | TGGGGGGGGT GTGGGAGGAC ATAAATGCTC | |
| | AGTCCATAAC AAGCACCCAA CATAAAAATG | |
| | GCTAGAACAG ATCACAAAAA AAAGGTCCTG | |
| | TATGGCTTTG GGGAAGGGCT CAACCCCAAA | |
| | ATATCTGAGA GCTCTGGAGG GGCCTAGAAG | |
| | TGGTAAATGA ATGAAAACGT GGTTACTCTC | |
| | CAGATCTGCC TTTCCCAAAT ATGGCCATTC | |
| | TTGGCTGAAT CAGAAATCAA AGGACAGGTT | |
| | ATTAATTACT AGCTCTAAGT TACTTACCAT | |
| | TTGCTGAGAC AGTTCAGAAA TCTGACTGCA | |
| | TCTCCTCAGA GATCTAGAAC ACAGTTCTCA | |
| | AATTCTAACT TACTTGTGAT ATACTTGTGA | |
| | ATGATAAAAA TCGCTACAGG TACTTTTATT | |
| | AATCTGAAAG AGTATTGAGA AATTACCTTT | |
| | CATTCTGACT TTTGTCTGGA ATGAAAATCA | |
| | ATACTTTTGC TATAATCGAT TACTGAAATA | |
| | ATTTTACTTT CCAGTAAAAC TGGCATTATA | |
| | ATTTTTTTTA ATTTTTAAAA CTTCATAATT | |
| | TTTTGCCAGA CTGACCCATG TAAACATACA | |
| | AATTACTAAT AATTATGCAC GTCACATCTG | |
| | TAATAATGGC CTTCATGTAA ACATTTTTGT | |
| | GGTTTACACA TAAAATCTCT AATTACAAAG | |
| | CTATATTATC TAAAATTACA GTAAGCAAGA | |
| | AAATTAATCC AAGCTAAGAC AATACTTGCA | |
| | ACATCAATTC ATCATCTGTG ACAAGGACTG | |
| | CTTAAGTCTC TTTGTGGTTA AAAAGGAAAA | |
| | AAAAAAAAAA GACATGTTGG CCAGATGCGG | |
| | TGGCTCACAC CTGTAATCCC AGCACTTTGG | |
| | GAGGCTGAGG TGGGCGGATC ACCCCTGGCC | |
| | TGCCCAACAT GGTGAAACCC CGTCTCTACT | |
| | AAAAACACAA AAATTAGCTG GGCGTGGTGG | |
| | CGGGCGCCTG TAATTCCAGC TACTCGGGAG | |
| | GCTGAGGCAG GAGAATTGCT AGAACCCAGG | |
| | AGGCAGAGAT TGCAGTGAGC TGAGATTGCA | |
| | CCATTGCACT ACAGTCTGGG CAACAAAAGT | |
| | GAAACTCCAT CTTAAAAAAA AAAAGACAAT | |
| | GTTCGTGGGT CCAAACAAGA CTTAATGGAA | |
| | GTGAGTCTAA AAATGAGCTA TGTGGGCCAG | |
| | GCGTAGTGGC TCCCACCTGT AATCCCAGCA | |
| | CTTTGGGAGG CCGAAGCAGG CAGATCATGA | |
| | GGTCAGGAGA TGGAGACCAT CCTGGCCAAC | |
| | ACGGTGAAAT CCTGTCTCTA CAAAAATTAG | |
| | CTGGGCGTGG TGGTGCCTGC CTGTAATCCC | |
| | AGCTACTCAG AAGGCTCAGG CAGGAGAATC | |
| | GCTTGAACCA GGGAGTCGGT GGCTAGAGTG | |
| | AGCCGAGATT TGCATCACTG CACTCCTGCC | |
| | TGGTGACAGA GCAAGACTCC ATCTCAAAAA | |
| | AAACAAACAA AAATAAAAGA TAAAAATGAG | |
| | CTATGTGAAT TAAAAGAGGT ATAACAATAG | |
| | ATAAACCATA TTTTATTTAA TTCCTAGTAA | |
| | TGAGTAATAT TTCCAAACTT CTGGAATGGG | |
| | CAGAAATTGC TAGTTGGCAT ATTTTTACCT | |
| | TTTATATTCA GATACATTAA AATTCTCAAA | |
| | AAAAAACACC TCAAAGCAGA TGATCCGCCA | |
| | TCTCCTTGGA TAATTTGTGT TAACTCAGGA | |
| | TAACAGAAAA CCAAAATTAT GAGTTACTGA | |
| | TGCAATATTC CTAAATGTAA AAATAATTAA | |
| | AGCTAATAGT AGATTCATCT TCCAATTTCA | |
| | TATCAGTCTT ACAAATAAAC TACATATATA | |
| | ACTTGCTTGC CTTCCCTTCT GAGGGATAAA | |
| | GCTGTTAGAA GAATTAAAAT CAGCATTCTT | |
| | GACTATTCAA CCAAGGGAGG GATAAATTAT | |
| | TACTCATTCT AGGGACATGG GCTCATAACT | |
| | ACTACATGTG TAAGGACATG AATTTACCCA | |
| | ATATTACAAT TTTTCCTTTT ATTAGTGTGT | |
| | ACAGTGGAAG AATAGACATG TTCACTCTGG | |
| | ACAAAAAAAA AATTATACTT ATCAGTTATC | |
| | AGAAGCACAA TGCTGAAGAC AGTAGTTCCA | |
| | TAACAATTTG AAGTATGTGA TCGAACTAGT | |
| | AGATTATCTT AGTAGTAGTG AATTATTGTA | |
| | AATGTTAGTA ATTTGGCAGC CACTGGGCAG | |
| | AAAAATAAGA ATTGAGGCTC AATATTGATA | |
| | TTAATGGTGG TGATTGACAC ATAAATTTTA | |
| | TCAAGTCTAC ACAATATAAA ATTACAGAAA | |
| | GGTAGAAGAG TATACCAGTA CAACTTCAAC | |
| | ATATCTTCAC TACAAGGGAG TAAAATGACA | |
| | TGGCCTAGTT ACTATCTAAT GAACTGCAGA | |
| | AAACTAAAAG AAAACTCCAA GGCAACTCTT | |
| | CTCTGCTGAT CTGGTTGGTC CTTTTCCTAC | |
| | CTTTTGCAAT ACCCAGATAC AAACAATGGA | |
| | TAGAAAACAA AGTAGACTTG TAGTATGCAG | |
| | GTCACAGTGC TAAATTCACA GAAAGAAACC | |
| | CCTGAACTGA ACTGCTCTAT TTCCTGGTGG | |
| | TCACAAAGAG TAATTCTGGT TTACACCTAC | |
| | AGATTGATGT CAATCTACAC CCTGTTGATA | |
| | ACAGTGTGGC CAAGGACAAA AAAAAGGTGC | |
| | TCCGTTTTAC CAATTCTGTA AAAAATTATT | |
| | GGCAGGGTAA GCTCGGCTAG GGCAGGATTA | |
| | CATTTCTAGG ACTACCATCC CCGAAATTTA | |
| | GAAGATATTA TATCCACATA AAGCATATCT | |
| | TTCACATTAA TTTGCAAAAA TCTAAAAGCT | |
| | TTTTCTTAGC TCAAGTGTGT CCAAGTTTAC | |
| | CCTGGCAGTT TAAAACGATA GTTACAAGCA | |
| | GCATGGGTTG TATCAGACAC ATTTGAGGGC | |
| | CAATTTCATG TAAGTGATAT TGGGCAAGTT | |
| | ACTTCAACTA TCTGTGCCTC CAAGGTCATA | |
| | CTAGTGTTTA TTTACCTAAA GGGTACCTGT | |
| | TATGTAACTT TAGGGTGTTT ACATTAGATA | |
| | ATGCCTGCAA AATATTTACT TCAACGCCTA | |
| | AAACATAGTT AAGTATTCAA TAAATACCTA | |
| | CTATTGTCAC TACTAACTTA AAAGTTTAGA | |
| | GATTAAGAGC AGAATCTGGG GTGAGACAAA | |
| | CTTAGGTTCA AATCCTAGTA TTGTTGGGTA | |
| | ATCTTGGGCA AGTTACTTAA CCTCTCTGAT | |
| | TTGTGTAATT TAAAAAATTA GTTAATATAC | |
| | ATAACAGGGC TTAGAAGAGT ATCTAGCACA | |
| | TAGCACCATT TAAGCATTTG TTATTGCTAA | |
| | CATGCAAACA ATTTAAGGGA AAGAAATTTT | |
| | TTAAAAAGGA AGAGGGATTT GCAAACTAAA | |
| | AACAATGAGT ATCTTATGTT CAAAGAAAAC | |
| | TAACAAACAG CCAGCTCTAG CAATAATTAA | |
| | ATTCACTATA TACTGGGGCA GGCATCACAC | |
| | CCCAAAGCTA AAAGCGTCTA CCTAGGCCAG | |
| | GCACGGTGGC TCATGCCTGT AATCCCAGCA | |
| | CTTTGGGAAG CAGAGGCGGG CAGATCGCTT | |
| | GAGCTCAGGA GTTCAAGACC AGCCTGGACA | |
| | ACATGGCAAA ACACCATCTC TACAAAAAAT | |
| | ACAAATATTA GGCCGGGCGC AGTGGCTCAC | |
| | GCCTGTAATC CCAGCACTTT GGGAGGCCAA | |
| | GGCGGGTGGA TCACCTGAGA TCAGGAGTTC | |
| | GAGAGTAGCC TGGCCAACAT GGTGAAACCT | |
| | CGTCTCTATT AAAAATACAA AAAATTAGCC | |
| | AGGCATGGTG GCAGGCGCCT GTAATCCCAG | |
| | CTACTCAGGG GGATGAGGTA GGAGAATCGC | |
| | TTGAACCCGG GAGGCAGAGG TTGCACTGAG | |
| | CCGAGATCAT GCCACTGTAC TCCAGCCCGG | |
| | GCAACAAGAG CGAAACTCCA TCTCAAAAAA | |
| | TAAATAAATA AATAAATAAA ATAAAGTACA | |
| | AATATTAGCC AGGGATGGTG GTGCGCACCT | |
| | GTAGTCCCAG CTACTTGGGA GGCTGAAGTG | |
| | GGAGAATCCC CTGAGCCTGG GGAGAATCAC | |
| | CCGAGCCCGG GAAGTCGAGG CTGCAGTGAG | |
| | CAGTGATTGT GCCACTGCAC TCCATCCTAG | |
| | GTGACAGAGT GAGACCCTGT CTCAAAAAAA | |
| | AGAAATTGGC AGAATTAAGT AAGTTGATGT | |
| | TTAGAGATGA AAAATCAACA TTTTTTCCTC | |
| | AGCAACTGAA TAAAAACAAC AGCCACTACC | |
| | ATTTTTTTGA GTACCTATTT GTAGCCTATT | |
| | TTTTAACTGG TATTACTCGA GAGAGAGAGA | |
| | GCTAGGTTCG AGACAGAGCT CCTTCTCTTA | |
| | ATAACTGTAT GACCTAGGGT ATGTCTGTTA | |
| | GCCTCTCTGA GGCTTCAAAG GTTCCTCATC | |
| | TGTAAAATGG TAATAATCAT ACCATTGCTA | |
| | CAGGGCTGTT TTGAAGACTA ATTAGGACTA | |
| | TGTAAGTAAA CATGATGATG GCTATTATTA | |
| | CTGTTCCCCG CCAGGGGCCA TGCAAGGGTT | |
| | GCTGATTCAC ATAGACTGTC TTATAATCCT | |
| | CTCAATAACT CCAAGAGGTA GCCAGCACCT | |
| | CAGATATACA TAAAATGACT TAAGCCCAGA | |
| | GAGGTGAAGT AAGTTGCCCA CAGCCACACA | |
| | ACTAGTAAAT AGCCCAAACA AGCTGGATTC | |
| | CCAGTTAGAC TCCGTTAATA GCACTGCTCT | |
| | TTACCTTAAG TCATTACAAT GCCTAATATG | |
| | AAATAGAATC GCTTCTTTCT TAGGGTTCAA | |
| | GTGGTTAATT ATTTAATGTA TTCATTCAAC | |
| | AAACCATCAT CGAGGACCTC TTACAAGCCA | |
| | AGTACTGTGC TAAGTGCTAG AGTTACGGCG | |
| | GTGATTCCTG CCCTTAAAAA GTTTTAGTGG | |
| | GAGAAACAAC AGGTAACCAG GTCATTGCCA | |
| | AAACAACAAA AATAATCATA ATAAAGCAGG | |
| | CTAAAGCATA TTTAACTGGC CGGGGTTTTG | |
| | ACTATTTTAG CAAGCATGAT CAGAACGGTT | |
| | GAGGAGGGAG GCCAGCAGCT TGGCCGGTTC | |
| | AACAAACAAG AAAAAACCAG TGAGGGTGGA | |
| | GCTAAGATAC CAGAGGCTGA TTACGGTTAA | |
| | GAATGTTCTT GAAGGTAAGG ACCAGATTCT | |
| | CATTTTCTAT ATCCTGGGGC ATCGGTCAGC | |
| | ATGGAATCTG GATTCTAGCA CATGTGAATT | |
| | TCGGCTTGAA ATGACCTAAT GCCTTTTCCC | |
| | TAGTTCCTTC GTGTGTCAAA TACGCATGGT | |
| | TACCGCTACC AGAGCTGTAG TGGGGCTTCA | |
| | ATGAGGCCAT GAGCATCTCC ATAAAGATGA | |
| | ACTACAGTGT GTGCAAAACT AAAGGCAAAA | |
| | CCTGGTCCCC ACACGCCCTC CCAGGTGGTC | |
| | GCTTTCCGTG CCGAGGCCCC TCCAGAGGTG | |
| | CCCCGAGAAC CTCACCATCG CACCCCAAAC | |
| | TTCCAGGGAA GGGCCTCTCC CGAGAAAGCC | |
| | CCCACGCCCC CACCCCGCGC CATCATTCCC | |
| | GAATCTGCCC TCGGCCCCTC CCCGCAGCAC | |
| | GCTCGCAGGC GGCACATGTC AACCAAAACG | |
| | CCATTTCCAC CTTCTCTTCC CACACGCAGT | |
| | CCTCTTTTCC CAGGGCTCCC CCGAGGAGGG | |
| | ACCCACCCCA AACCCCGCCA TTCCGTCCTC | |
| | CCTGCCGCCC TCGCGTGACG TAAAGCCGAA | |
| | CCCGGGAAAC TGGCCGCCCC CGCCTGCGGG | |
| | GTTCCCTGGG CCCGGCCGCT CTAGAACTAG | |
| | TGGATCCCAA TTGAAGGCCT GGTCTAAATG | |
| | ACTCCAAAAT CACCACTTAA TTCAAGAGAC | |
| | TGATTTCCCT GAGTCAGGCC CCTTAAAGCA | |
| | GCTATTTCAA TGGGACAGGG AAACAACCCT | |
| | AGGATCTGGA TTAGAATCAC TTGGGGGCTG | |
| | CCACACCCCC AGGGCTCTGA TCCTGCCCTT | |
| | CTCCCACACG CACATTCACA TACTGCTGCA | |
| | GTGACCTTCC ATTTCTAATG GGTTCCTGGG | |
| | CCATCTGTCA GGTATAGGGA ATGGAAAAGG | |
| | GGTTGGGGAG GCTCTGCTTC AGAAAGTTTG | |
| | TGTCAGGGGC TCCCAGAGCC TCCACAGATA | |
| | GATAGCAGGG GTCCCCACCC TACCATGGCA | |
| | GCTATAAATG TGATCAACAT TTATTGGCCT | |
| | AGGATACAGC AGTTAGCAAA ATGCCTGATG | |
| | TAGTTCCCAC TCCGTGGAGG TTGCAGGCTA | |
| | GCCAAGAAGT CATGAGTTCA GCAACCCTTA | |
| | CGCACCAGTG GGATGAGATT GGACCAGGCC | |
| | GAGGGTAGTC TTGGGAACAC TCAGCATTTG | |
| | TCTGAGGGCC AGAAGAGGCT GCTTGCCCTC | |
| | AGACAGGAGG TCAGCATCTT TATTGTAGCC | |
| | CATGACACCT CTACACCATT GCTCTTCTGG | |
| | TCTTATGGAA GACATCTTTG GGCCTGATAA | |
| | CAGCGGAGTC TGTGTCCCAC TTGTCCAGGC | |
| | TGGAGTGCCA CATCAGGCAC ACTCCAGTTG | |
| | CAGGGACAGC ACAGACAAGT TTCAGGAAGG | |
| | CTGGTGGCCT CCAGGAGGTT AACCTTATAA | |
| | GGCCAGATTG TAACCTAGTT GAAAAACATA | |
| | CACATGCCAT GATAATAAAA GAACCTAGGC | |
| | ACCATTACAA GAGAAAAAAT CATTTTTGTA | |
| | GATACGAGCA TGGATTCTTG GGTGGGTCAG | |
| | ACACACTGGG CTTGTGCTCT GACTGCACTG | |
| | TCTCCCCTAC CTGACCTTGG GTAAACCATA | |
| | AGACTGCTGC ATGACTCAGT GTCCACCCCA | |
| | AAAAAGTACC GGTAGATATT GGCCACAGTA | |
| | GATATCAGCT AGAGTGGACT CTCATGACAA | |
| | TGAGGGGAGA TGTATTCCCC ATCTTAGGCA | |
| | CCTGGGACTC TACCTTCCAT CTTCTGCTCC | |
| | GTGTCTCTCC ATCCCCAGGC TCTTCAGAAC | |
| | TCAGGGAGTC CAGAATGTCA GCTCCCAGAT | |
| | TTCAGCCTTC AGAAAGGAAA CCCATTACCG | |
| | TTCAGTTGAA CAAATGTTGT CTGAGCCCCA | |
| | GATCTGGGCT CAGAGGCCAT CTAGGCTATG | |
| | AGACAAGAGG GGAACAAAGC ACCGTCTGCA | |
| | CTCACTCACC ACACTCACTT GCTGTCCCAG | |
| | GTCACATCCA TCGGGTAGAG AATCTAAGAG | |
| | GCTGAGCTAG CTCCCGCCAC CAGCCCAGCC | |
| | CACCCCACCT GGCCCCTTCC TTCCTTCTAC | |
| | AAAATATGCA CCACCTGTCA AAGGGTGGGC | |
| | AGTGCCAGGC CTGCATACAG AGCACTGAGT | |
| | GTAAAAGCAG ACATGGACCC TGACCTCCAG | |
| | GAGCTTCCAA TTTTCTTGAA GAGACAAATC | |
| | AGCTGGCATT TCAGTCCAGT GTGATCTGCT | |
| | CTTGGTGAGC ACAGACCTAG GGAGTTGGGG | |
| | CAGCTTCCCA GAAGAACTGC AGTCCAGGCT | |
| | GAGGGCAGAG AAATGAGGGG AATGGCGAGG | |
| | AATTGGGGAG CAGGGGGGAG CTCAGTAGAG | |
| | AGCCAAGGGC GGGAGGTGAG AAGTCCGTGT | |
| | TGGGCCAGGA GCTACCCTCC GGTGGCCACA | |
| | GCCGAAGTCG AGGATGCCTT TGGAACTCAT | |
| | CCCCACTTCT CTCTTTCTGT ATGTAGCCGT | |
| | CCAAGAACAA GTCACCTCCA AGTGTAGCCG | |
| | GATCAAGGCA AGCCCCCCAT CTAGCAAGCA | |
| | CTTGATGCCA CCCAGAACTG GGCTTCTTCA | |
| | GAACAATCTG AGTCCAGGAA TGATCCCACT | |
| | CACCAGGCAC CAGAGCTGCG AGGGCATGGG | |
| | AGTGATCTCA CCAACTCTGG GGAAGCGGCA | |
| | AGGAATTTTC ACCTCCAGCC CCCAGTGTCC | |
| | CATCCTCTCA CACTCAGGCC AGACTCCCCT | |
| | GGGCAGACTT GACTCTGTCT GCCAGCATAT | |
| | GCAGAGCCCC AAGGCCACCC CACCAGAAGT | |
| | GCCCCTGCCT GGGTTCTGTC CCAGCTCCCT | |
| | GGGCACCCAG TCCTTGAGTC CCCACCAGCT | |
| | CAGACGGCCT AGTGTGCCAA GAATGCCCAC | |
| | TGCGTTCAAC AATGCTGCAT GGGTCACAGC | |
| | GGCAGCAGCT GTGACCACAG CAGTTTCGGG | |
| | GAAAACACCC CTCAGCCAAG TGGATAATAG | |
| | CGTTCAGCAG CACTCACCTT CTGGCCAGGC | |
| | CTGCCTTCAG AGGCCATCTG ATTGGGAGGC | |
| | ACAAGTGCCC GCTGCGATGG GAACACAAGT | |
| | GCCCCTGGCC AACAACCCCA GCTTCAGCCT | |
| | GCTGGGCAGC CAGAGCCTCA GGCAGAGCCC | |
| | GGTACAGGGC CCGGTGCCTG TAGCAAACAC | |
| | CACCAAGTTC CTCCAGCAGG GTATGGCCAG | |
| | CTTTAGTCCC CTGAGCCCCA TACAGGGCAT | |
| | CGAGCCACCA AGCTATGTGG CTGCTGCTGC | |
| | CACCGCTGCT GCTGCTTCTG CCGTTGCTGC | |
| | CAGCCAGTTC CCAGGTCCGT TCGACAGAAC | |
| | GGATATTCCC CCTGAGCTGC CACCTGCCGA | |
| | CTTTTTGCGC CAGCCCCAAC CCCCACTAAA | |
| | TGATCTGATT TCGTCACCTG ACTGCAATGA | |
| | GGTAGATTTC ATTGAAGCTC TCTTGAAAGG | |
| | CTCCTGTGTG AGCCCAGATG AAGACTGGGT | |
| | GTGCAACTTG AGGCTGATCG ACGACATTTT | |
| | GGAACAGCAT GCTGCTGCTC AAAATGCCAC | |
| | AGCCCAGAAT TCTGGGCAAG TCACCCAGGA | |
| | TGCTGGGGCA CTTTAAATCT GAGCAGGATG | |
| | CCCATAGAAA CCCCCATGGT GACATCACTC | |
| | TAGGAAGTGG TGTCGATCCA TACCCGCAGT | |
| | TGTCTCCCGT TACAATTTGA GTGGTGTTGT | |
| | CAGCCCATGC TTATCCCTCT CTCTACCTGT | |
| | GACAAAATGG AAAGCTGGTG ATTTTTCAAG | |
| | CTACGTGTAC ATATTTGAAA ATTTTGTAAA | |
| | TGGTTTTCCT AAACATTAAT GACAGAAGTA | |
| | TTTATACTTC ATTTTGTGAC TTTGTAAATA | |
| | AAGCGACGGC TTTTGTTTCA GTAGAGTTGT | |
| | GTTTACTATG CATTGTTTTG TGTTTATTAT | |
| | ACAATGTTAC AAATATGCAG ACCGTGTTGT | |
| | TTGCTCCAGT GATACCTTGT TAAGCTAGGT | |
| | GGCTGAGTCG CTTATGGTTT TAATGCAATG | |
| | AGCAATGTGG ATATGACCAA GAGTTGTTGT | |
| | GCAAGTTGAC AAATGCCAAA TAGAAAACCA | |
| | CTTGGCCATT TATTTCTATG TTCACTAAAA | |
| | ATCCTATTGC CTTGTGTGAT TCTTAATCTC | |
| | TTTTGCGAAC CTTTCAGTCT CCGCTAGCTC | |
| | TTTCCTAATG AGCTTTACAG CAGAAGCTGT | |
| | TTTATCGTTA AGTGCCCCAC AGAGACACTT | |
| | TACCAGGAGG CTGGGAGAGT TCTCCAGATT | |
| | TGGGAGAGGC GCAGAGACAG TGTGTGAGCC | |
| | GAGCCCTGTC TCAGCAATCC ACCTGGAGGA | |
| | GCTAGAGTAT CCTCCTCCCT TTACCATTCA | |
| | GACCGAGAGA AAAAGCCCAG CTTGTGTGCA | |
| | CCCTCGTGGG GTTAAGGCGA GCTGTTCCTG | |
| | GTTTAAAGCC TTTCAGTATT TGTTTTGATG | |
| | TAAGGCTCTG TGGTTTGGGG GGGAACATCT | |
| | GTAAACATTA TTAGTTGATT TGGGGTTTGT | |
| | CTTTGATGGT TTCTATCTGC AATTATCGTC | |
| | ATGTATATTT AAGTGTCTGT TATAGAAAAC | |
| | CCACACCCAC TGTCCTGTAA ACTTTTCTCA | |
| | GTGTCCAGAC TTTCTGTAAT CACATTTTAA | |
| | TTGCCACCTC GTATTTCACC TCTACATTTG | |
| | AAATCTGGCG TCTGTTTCAA GCCAGTGTGT | |
| | TTTTTCTTCG TTCTGTAATA AACAGCCAGG | |
| | AGAAAAGTGC CTCTATGTTT TTATTTTTCA | |
| | AGGGAGTATT CAGTACCTAC AAACCCAAGT | |
| | CAGGAAGCCT GCTAGTGGCT TTGGTTCTTT | |
| | CAGAGGCTGC TCGATGCCTT GTGTGTCAGA | |
| | AAGAAAGATT CAGCAGTTTT GCATCATGGC | |
| | AAAGAAGCCT GTTATTTTGG GGCTCAGCCC | |
| | CTCATTTTAT AGAGGATGAA ACAGAGGGGG | |
| | ATGGGAGGTC ACAAAGACAA CTGCCCCGGG | |
| | AGCAGGTGTG GGGGAGACTT GCCCTGAGGG | |
| | TCTAGACGCT CTGCACCACC GTCCTGTCTC | |
| | CCTTGCTGAA GACCACACAT GCCCTTCTTT | |
| | GACCAGACCC TGCCACCTGA TAGGCCAGGA | |
| | CCTGGTAGGC GGGTACCCAG GTTTCATGGA | |
| | TGGAACCACA TCTCCCCAAA AGTGGGGAGG | |
| | TAGCTACTGG GATGCACGCC TCCCGCCATG | |
| | TGCTATAGGA GAGCAGCTGA AGCAACAGTT | |
| | GGGATCAGAT GTAGTCACAA TTGAATGCAT | |
| | CATCACATTT ATCCCTCTAA GTGGCTGGGA | |
| | GAGTTGATAT CCTCATCCCT AAGGTACAAA | |
| | ATGTTCCAAT TTGATCAGTG GCTTTCAGGA | |
| | GCTGAGAAAG GCATGTGCTC TGAGGCAGAG | |
| | CTGTTATGTC CCGCAGAGCC TAAAAATGCT | |
| | CTAAGAACAT GCTCCCTGCC AAAATTCTCA | |
| | ATGGCTGTGA CAAGGGACAA CGATCGACCA | |
| | ATGGGGGTGG AAGCAGACCT CCGCAGTCCA | |
| | GGGGCCAGAG CTAGGACAGA GGGGTCGGAG | |
| | AAAGAGTCAT TTTCCCAACA CTCCAGCTCT | |
| | TGGCCAGTCC TCACACAGTC CCCTCCTGCT | |
| | TCCTGCTGAG AGAGATATCC TCATAGGTCT | |
| | GGGTAAAGTC CTTCAGTCAG CTTTCATTCC | |
| | CTGTCACCAA CTTTGTCTCT GTTCTCCCTG | |
| | CCCGTCTCAG GCAGCACTCC TCAGGAAACC | |
| | TCTCCAAGAG CCAGCCTCAC TGCAGCGCCC | |
| | ACTATTGTCC CTCTGCCTCA AGTGTCCCAT | |
| | CCATGCCAGG CCCCAGGCAG GCTGCAGCTT | |
| | TCCCTCAGGG CCACACCAAA GCACTTGGGC | |
| | TCAGCTGTGC TGTCCCCCTC CATCACTGAG | |
| | CTCAGGGGCA GCAGGGGTGG GGTGCCAGGA | |
| | GGCCCATTCA CCCTTCTCTG GCTCTGTGTT | |
| | GGACCCACCT GCCCAGCCAC TGCTGCTTAG | |
| | AACCTACCCG CTGGGAAAAT GAAGCCCTCC | |
| | CGGAGGGGCC ACCTCAACCT GAGAGCCTCA | |
| | CGGATCACAG TTGTCCCCAC TCAGCTCTGC | |
| | CAGCCCTCAG AGACCCATAG ATAAAAGCTG | |
| | AGCTTGGCTC GCAGAGCTGG TTCCATCTTC | |
| | CATTCCCAGA GGGTTCAACT TCCTACCCCA | |
| | ACCACACAGG GAACCTCAAG GCTGAGCCAG | |
| | TGTGGGCTGC AGTGCAGACC AGCTTCCTGG | |
| | ACACGTCCTG CCACCTGACC CCAGGCTGGC | |
| | CTCACTGCCC CTGGCACTCC TGACCCTATC | |
| | CTCATTCCTC CTGGCAGTGC GTGTTCTGCC | |
| | ATTCCGCTTT CCCTTAGCTG TCCTCTCACT | |
| | GTACTGTCAG CTTCTCCTTT TCCAGGTGCC | |
| | CCCCAGGGGC TTTCCACATG ACCCTGTCAC | |
| | CCCACAGCCC ATCCAGCACC AATTCCAGCT | |
| | CTCTGCCACC CTTCAAAGGA GTGACAGTGC | |
| | CCTGCTTCAC CTCCCACTCA CCCCTCAACC | |
| | CAGAGCAATC TGGCTCCAGT CTTGCCTCCT | |
| | TCCCCCTAAG TACTCTAGTC ACAGTTCCAA | |
| | ATTCCTCCTG GTCATAAAGC CAAATGAAGC | |
| | TTCCTGGTCC TCAGCGGACT TGCCACTTCA | |
| | GCAGTACTGG ACTCTCTCCT CCCAGAAACC | |
| | TGTTTCCCCT TGGCTCCTGG AGCCCACACT | |
| | CTGCTGGAAT CCTTCTGCCT CTCTGGCCTG | |
| | TAGCCTGGCC CTCTCTCCCA ACCTGAGGTC | |
| | CATTCTCTCC TGCTCCTCCA CAAGATGTTG | |
| | CTCCTTCCAT TACTTCCTCC CTCTCAACCA | |
| | AAGCTCCTTC ATTAGCTCTT TATCTTCTGG | |
| | TTTCTTCCCC TGGGCAGACG AATGGATTCA | |
| | AGAGCCTGTG GCCCAGCAGC CCAGCACTCC | |
| | AGGATCTCAG CACTTCAGCA TCCCAGTACC | |
| | CTAGCATCTC AATACCCCAG CACCCCAGCA | |
| | CCATAGTATT CCAGCACCCC ATTGTCCAAG | |
| | CATCTCAGCA CTCCAGCATC CCAGCACCCC | |
| | AACACTCCAG CAGCCCAGAA TCTCAGCACC | |
| | CTAGCACTGC AGCATCTCAG GACCCCAGCA | |
| | CTTCAGCATC CCAGCACACT AGTACTCCAG | |
| | CATCTCGGCA CCCCAGCACC TAGGCATCCC | |
| | AACACCCAGC ACCCCAGCAC TTAAGCATCC | |
| | CACCACTACA GTATCTCAAC ACTCCAGCAC | |
| | CCCAGCACCA TAGTGTTCCA GCACCCCAGC | |
| | ATCCCAACAC CCCAGCACTT AAGCATCCCA | |
| | ACACCTCGGC ATCCCAACAC CCCAGCACTG | |
| | CAGCATCTCA GCACCTTAGC ATCCCAGTGC | |
| | CCTAGCATCT CAATGCTCCA GCACACCAGT | |
| | ACTACAGTAT TCCAGCACCC CAGCACTCCA | |
| | GCATCTCAGC ACTGCAGCAC TGCAGCACTC | |
| | CAGCATCCCA AAATCCCAGC ATCCCAACAC | |
| | CCCAGCAGAC CAGCAGACCA GCATCTCAGC | |
| | ACCGCAGCAT CCAAGGACTA TCCCAGCATC | |
| | CCAGCAACCC AGCACCTCAG CATCCCAACA | |
| | CCCCAGCATT TCAGCATGGC AACACCCCAG | |
| | TACCCCAGCA CTTCAGCACC CCAGTATCCC | |
| | AGCATCTCAG CGACCCAGTA TCACAAAACC | |
| | TCAGCATCCT AGCACCCCAG CACCCCAGCA | |
| | CCTTAGCACC TTAGCATCCC AGCATCTCAG | |
| | CGCCTCAGCA TCTTGATATT CTGGCTGAGG | |
| | TCAGCGTGGT GTATCTAGTC AGGGTCCTAA | |
| | CTTTCACTTC GCAGGGAAAT GCTGCTGGAC | |
| | TGGGTCTCAT GTTGGGCTGA AGCTCTCTAG | |
| | ACCCCTTGAA GACAGCATAA AAGAGCTTGG | |
| | AGACGCTGGG TGTCCCCCAT GGAAGAGTTC | |
| | ACTCTCATCC TGCTTTGACA ACAGCCTTCT | |
| | CTGGGGTCCC TCACGGGCCC CTCTTTCTTA | |
| | CTGCAAGTTT GTCTCTGAGA AGACTGTGAT | |
| | GCAGAAGTCA CTCAGCTGCC TGTGGCTCCT | |
| | GAAGAGCTGA AGGTGGAGGC CTGTAGGCCT | |
| | CCCTATGAGA GGCGCAGAAA AAACCATGAT | |
| | TGCTAGTGGG GAGGTGCTCC CTCTACAACC | |
| | CACTCCATAA TCTGCCCCCG CCCAGCTCTG | |
| | AGGCCAGCCC CAGGGGAAAA TGCCAGATCC | |
| | CCAGGGAGGT GTGTGAGACC TCAGGGGCTC | |
| | CCTCCTCCCT TACAGCAGGC TCAGGCCCCT | |
| | GGGGGCCTCA GGGCCAAGGT CTGTGGGTAA | |
| | GCTACTATCT CTCACTTGTC CTCTAGCCAC | |
| | AAAAGCCAGG GAGATCTGGC AATGGACATG | |
| | AGGTTCTGAA GAAGCACATA TGACTGGCTT | |
| | CCTAATGCGT GGTTGTTCAG TGATTCAATA | |
| | AACACGCATG GGCCAGGCAT GGGGAAATAG | |
| | ACAAACATGA TCCCCAACCT CTCCCAGAGT | |
| | GAACTGGGAG GGAGGAGTGT TCATCCCTCA | |
| | GGATTACACC AGAGAAACAA ACCAGCAGGA | |
| | GATATATATG GTTTTGGGGG GTCAAGAAAG | |
| | AGGAAAAACC TGGCAAGGCA AGTCCAAAAT | |
| | CATAGGACAG GCTGTCAGGA AGGGCAGCCT | |
| | GGAACCTCTC AAGCAGGAGC TGATGCTGCA | |
| | GTCCACAGGC AGAATTTCTT CTTCCTCGGG | |
| | GAAATCTCAG CTTTGTTCTT AAGGCCTTTC | |
| | AACTGATTGG CTGAGGTCTG CCCCTTCCCC | |
| | CACATTCTCC AGGATAATCT TCCTTACTTA | |
| | AAGTCAACTA TTAATCACAG CTACAAAATC | |
| | CCTTCACAGC TACACATAGA TCAGTGTTTG | |
| | ATTGACGAAC AGCCCCTACA GCCTAGCCAA | |
| | GTTGACACAT AAAACTAACC ATCACAGGGG | |
| | GACAAATGAT GTAAACACAT CAACAAATAA | |
| | AACAGTAACA AGTTAAGGTC TATGGAAAAA | |
| | ACACAGAAGG GGCAGAGAGA AAGAAAGCAA | |
| | GAAGGAGAGT CCCAGTTTGC TAGGGCTTGT | |
| | GGGAAGTGGG GAGCAGTTCT CTTTAGCTAG | |
| | GATATTTGGG AAAGGCATAT CTGAAGGAGT | |
| | GATATTTGAG CTTAGATTAA AAGATGGGAA | |
| | GGAGCAAGCC ATGCAAAGAG CTAGGATGTT | |
| | CCAAGCAGAG ACGGAACAGC AAGTGCAAAT | |
| | GTCAGGAGGA ATAGAAGGAG GCTGGTGGGT | |
| | GGGGTCCAGT GAGCAAGAGG AGGGCAGGCA | |
| | GGAGAGGGGA TGGGGAGGTG GGCAGGCCCA | |
| | GACCACCCAG GGCCCTGGAG ACTATCCTGA | |
| | TCCAACAAGG GAAGCCTTGA GTCACTTCAG | |
| | TGTCCATGTG GAGAATGGAC CTCAGACTGA | |
| | ATGAGGGAGG CAGTAAGGAG GGCCTCTACC | |
| | TCCAGGGCTT CGCCCTGTGG ACTGCGCATA | |
| | GACATCTCCA ACTCAGAAAG TCTGAACCAA | |
| | ACTTTCCATA GTTCCCCCAA GTCTGGGCAT | |
| | CCTCCTACTC AGTGAAAGGC AGCCATCACA | |
| | CCTCCCTGCC CTGCTCCCGG ATGCCCCAAA | |
| | TCCTCTTGGT CTCCAAGTCC AGAACCTGAG | |
| | ACTTGTCCTT GATGTTTGTC TTTCCCTCAC | |
| | CCTTTCTGTA TTCTGGGAAG ATGGGTTTTT | |
| | TTCCCCCAGA TGAATCTGTA AAACTTCTGT | |
| | GATCACAATA AAAATTCTGG CAGTATTATT | |
| | TTCTGGAACA TGACAAAGTG ATTCAAAATT | |
| | ATTTATCTGG AAGACTACAA AACAAGAATA | |
| | GCCAGGAAAT TTCTAAAAAG AAAGAAGAAG | |
| | GAGGAGGAGA AAGAAGGAGG AGGAAAAGGA | |
| | GGAGAAGAAG AAAAGAAAAA GAACCAAGAA | |
| | AGGGTTCTAG CTCTACCAAA TATTAAAACA | |
| | TATCATGAAG CTATTTAAAA CAATATGGTT | |
| | GTGGATACTG AAAAAGATGT GAATAAAGTG | |
| | GAAGGAAAAT AAATAGAAAT GCACATGGGG | |
| | ATTGAGACTG TGAAAAAGGC AGCATCTCAC | |
| | ATCAGTGAGG GATGTTCAAC ACCTGGTGTT | |
| | GGGAAAACTG GCTAGTCATT TAAACCAAAC | |
| | AACTGGGTCC TCTACCTCAC TCCTGACATT | |
| | AAGATACATT TAGATGATTC AAAGAGTAAG | |
| | ACAGAAAAAA TAACACGTGA AAACACTATC | |
| | AGAAAACAAC GTGGGCCAGG TGTGGTGGGT | |
| | CACGCCTGTA ATCCCAGCAC TTTGGGAGGC | |
| | CGAGGCAGAC AGATCACCTG AGGTGGGGAG | |
| | TTCAAGACCA GCCTGACCAA CATGGTGAAA | |
| | TCCTGTCTCT ACTAAAAATA CAAAATTAGC | |
| | TGAGCGTGGT GGCGCATGCC TGTAATCCCA | |
| | GCTACTCAGG AGGCCGAGGC AGGAGAATCA | |
| | CTTGAACCTG GGAGGCAGAG GTTGTGGTGA | |
| | GCCGAGATCA CGCCATTGCA CTCCAGCCTG | |
| | GGCAACAAGA GTGAAAATCC ATCTAAAAAA | |
| | AAAAAAAAAA GCCAAGGTGG ATATTTTTAT | |
| | AGTATCAGGG TAGATCAAGC TTCTCCAATC | |
| | ATGACATGAA ACCCAGAAAC CATAAAAGAA | |
| | AAGAATGATA AAATTGCCCA CGTAAAGTAA | |
| | AAAGCTTGCA CACAGAAAAA CACCATACAG | |
| | GTTACAAGAT GAGCAGCAAA ATCAGAGAAA | |
| | AAACATTGCA ATTCAGGACA CACAGAGGCT | |
| | ATTGTTCCTA ATATTTAAAA ATAAAAGTAG | |
| | TGGATTGTCT ACAAAAAGAT GAAGACAAGA | |
| | ATTTCAGAAA ACCAAATACT GCATGTTTTC | |
| | ACTTACAAGT GGAAGCTAAA CACTGAGTAC | |
| | ACGTGTACAC AAAGAATGGA ACCATAGGCC | |
| | AGGCACCGTG GCTCACGCCT GTAATCCCAG | |
| | TACTTTGCGA GGCCGAAGCG GGCGGATCAC | |
| | CTGAGGTGAG GAGTTCGAGA CCATCCTGGC | |
| | CAACATGGTG AAACCCAGTC TCTACTAAAA | |
| | ATACAAAAAT TAGCCGGGCG TGGTGGTGGG | |
| | TGCCTGTAAT CCCAGCTACT CGGGAGGCTG | |
| | CGGCAGTAGA ATCGCTTGAA CCCTGGAGGT | |
| | GGACCTTGCA GTGAGCCGAG ATCGCACCAC | |
| | TGCACTCCAG CCTGGGCAAC AGAGTGAGAC | |
| | TCCATCTCAA AAAAAAAAAA AAGGAATAGA | |
| | ACAATAGACA CTGGGGCCTA CTTGAGGGAG | |
| | GAGGGTGAGG ATCAAAAACC TGCCTATCAG | |
| | GTACTATGCT TATTACCTGG GTGGTGAAAT | |
| | AATCTGTACA CCAAACCCCA GTGACATGCA | |
| | ATTTACCGAT GTAACAAACC TGCCCATGTA | |
| | CCCGCTGAAC CTAAAATAAA AGTTGGAAAA | |
| | AAATATAGAA ATTTTCTTTG TAATAGCCAA | |
| | AAACTGCAAA CAGCCCAGGT GTCTATTAGT | |
| | AGAATGCATA AACAAACTCG GGCATGTTCA | |
| | TACAATGTAA AACTACTCAT CAATAAAAAG | |
| | TGATACTTCT CAGCAATGAA AAGAAACTAG | |
| | CTACTGATAC CAGCTACAAC ATGGATGGAT | |
| | TTCAAGTGCT TTATGATGAG AGCAAGAAGC | |
| | CAGACACAAA AGTGTCTATA TATATATACA | |
| | GTATATATAC GTATATATAC ACATATATAC | |
| | AGTATATATA TACATATACA TGTATATATA | |
| | TACTGTATAT ATACTGTATA TATATACACA | |
| | GTATATATAT ACATATATAC AGTGTATATA | |
| | TACTGTGTAT ATATACATGT ATATATACTG | |
| | TGTATATATA CATGTATATA TACTGTGTAT | |
| | ATATACATGT ATATATACTG TGTATATATA | |
| | CATGTATATA TATGTATACT GTATATATAC | |
| | TGTATATATA TATACACATA TATACAGTAT | |
| | ATATATACAG TATATACTGT ATATATACAG | |
| | TATATACGTG TATATATACA TATATACAGT | |
| | ATATATGTAA ATATACATAT ATACAGTATA | |
| | TATGTAAATA TACATATATA CATGTATATA | |
| | TATACACTAT ATATATACAT ATATAGTGTA | |
| | TATATACATA TATACATGTA TATATTTACT | |
| | ATATGATTCC ATTTATATAA AGTGCCAAAA | |
| | CAGTCAAAAA TAATCTATGT GGAAAAAATC | |
| | AACAAAGGGA TCCCCCGGGC TGCAGGAATT | |
| | CGATGGCGCG CCTTAATTAA AATTATCTCT | |
| | AAGGCATGTG AACTGGCTGT CTTGGTTTTC | |
| | ATCTGTACTT CATCTGCTAC CTCTGTGACC | |
| | TGAAACATAT TTATAATTCC ATTAAGCTGT | |
| | GCATATGATA GATTTATCAT ATGTATTTTC | |
| | CTTAAAGGAT TTTTGTAAGA ACTAATTGAA | |
| | TTGATACCTG TAAAGTCTTT ATCACACTAC | |
| | CCAATAAATA ATAAATCTCT TTGTTCAGCT | |
| | CTCTGTTTCT ATAAATATGT ACCAGTTTTA | |
| | TTGTTTTTAG TGGTAGTGAT TTTATTCTCT | |
| | TTCTATATAT ATACACACAC ATGTGTGCAT | |
| | TCATAAATAT ATACAATTTT TATGAATAAA | |
| | AAATTATTAG CAATCAATAT TGAAAACCAC | |
| | TGATTTTTGT TTATGTGAGC AAACAGCAGA | |
| | TTAAAAGGCT AGCCTGCAGG AGTCAATGGG | |
| | AAAAACCCAT TGGAGCCAAG TACACTGACT | |
| | CAATAGGGAC TTTCCATTGG GTTTTGCCCA | |
| | GTACATAAGG TCAATAGGGG GTGAGTCAAC | |
| | AGGAAAGTCC CATTGGAGCC AAGTACATTG | |
| | AGTCAATAGG GACTTTCCAA TGGGTTTTGC | |
| | CCAGTACATA AGGTCAATGG GAGGTAAGCC | |
| | AATGGGTTTT TCCCATTACT GACATGTATA | |
| | CTGAGTCATT AGGGACTTTC CAATGGGTTT | |
| | TGCCCAGTAC ATAAGGTCAA TAGGGGTGAA | |
| | TCAACAGGAA AGTCCCATTG GAGCCAAGTA | |
| | CACTGAGTCA ATAGGGACTT TCCATTGGGT | |
| | TTTGCCCAGT ACAAAAGGTC AATAGGGGGT | |
| | GAGTCAATGG GTTTTTCCCA TTATTGGCAC | |
| | ATACATAAGG TCAATAGGGG TGACTAGTGG | |
| | AGAAGAGCAT GCTTGAGGGC TGAGTGCCCC | |
| | TCAGTGGGCA GAGAGCACAT GGCCCACAGT | |
| | CCCTGAGAAG TTGGGGGGAG GGGTGGGCAA | |
| | TTGAACTGGT GCCTAGAGAA GGTGGGGCTT | |
| | GGGTAAACTG GGAAAGTGAT GTGGTGTACT | |
| | GGCTCCACCT TTTTCCCCAG GGTGGGGGAG | |
| | AACCATATAT AAGTGCAGTA GTCTCTGTGA | |
| | ACATTCAAGC ATCTGCCTTC TCCCTCCTGT | |
| | GAGTTTGGTA AGTCACTGAC TGTCTATGCC | |
| | TGGGAAAGGG TGGGCAGGAG GTGGGGCAGT | |
| | GCAGGAAAAG TGGCACTGTG AACCCTGCAG | |
| | CCCTAGACAA TTGTACTAAC CTTCTTCTCT | |
| | TTCCTCTCCT GACAGGTTGG TGTACAGTAG | |
| | TAGCAAGCTT AAGGATCTAG ACTGCCATGG | |
| | GCGTGCACGA GTGCCCCGCC TGGCTGTGGC | |
| | TGCTGCTGTC CCTGCTGTCT CTGCCCCTGG | |
| | GCCTGCCTGT GCTGGGAGCC CCTCCCCGGC | |
| | TGATCTGCGA CAGCCGGGTG CTGGAAAGAT | |
| | ACCTGCTGGA AGCCAAAGAG GCCGAGAACA | |
| | TCACCACCGG CTGCGCCGAG CACTGCAGCC | |
| | TGAACGAGAA TATCACCGTG CCCGACACCA | |
| | AGGTGAACTT CTACGCCTGG AAGCGGATGG | |
| | AAGTGGGCCA GCAGGCCGTG GAAGTGTGGC | |
| | AGGGCCTGGC CCTGCTGTCC GAGGCCGTGC | |
| | TGAGAGGGCA GGCCCTGCTG GTGAACAGCA | |
| | GCCAGCCCTG GGAGCCTCTG CAGCTGCACG | |
| | TGGACAAGGC CGTGAGCGGC CTGCGGAGCC | |
| | TGACCACCCT GCTGAGGGCC CTGGGCGCCC | |
| | AGAAAGAGGC CATCAGCCCC CCTGATGCCG | |
| | CCTCTGCCGC CCCTCTGCGG ACCATCACCG | |
| | CCGACACCTT CCGGAAGCTG TTCCGGGTGT | |
| | ACAGCAACTT CCTGCGGGGC AAGCTGAAGC | |
| | TGTACACCGG CGAGGCCTGC CGGACCGGCG | |
| | ATCGCTGAGG ATCCCCATCC AGCTTGGCCA | |
| | GACATGATAA GATACATTGA TGAGTTTGGA | |
| | CAAACCACAA CTAGAATGCA GTGAAAAAAA | |
| | TGCTTTATTT GTGAAATTTG TGATGCTATT | |
| | GCTTTATTTG TAACCATTAT AAGCTGCAAT | |
| | AAACAAGTTA ACAACAACAA TTGCATTCAT | |
| | TTTATGTTTC AGGTTCAGGG GGAGGTGTGG | |
| | GAGGTTTTTT AAAGCAAGTA AAACCTCTAC | |
| | AAATGTGGTA TGGAATTCAG TCAATATGTT | |
| | CACCCCAAAA AAGCTGTTTG TTAACTTGCC | |
| | AACCTCATTC TAAAATGTAT ATAGAAGCCC | |
| | AAAAGACAAT AACAAAAATA TTCTTGTAGA | |
| | ACAAAATGGG AAAGAATGTT CCACTAAATA | |
| | TCAAGATTTA GAGCAAAGCA TGAGATGTGT | |
| | GGGGATAGAC AGTGAGGCTG ATAAAATAGA | |
| | GTAGAGCTCA GAAACAGACC CATTGATATA | |
| | TGTAAGTGAC CTATGAAAAA AATATGGCAT | |
| | TTTACAATGG GAAAATGATG GTCTTTTTCT | |
| | TTTTTAGAAA AACAGGGAAA TATATTTATA | |
| | TGTAAAAAAT AAAAGGGAAC CCATATGTCA | |
| | TACCATACAC ACAAAAAAAT TCCAGTGAAT | |
| | TATAAGTCTA AATGGAGAAG GCAAAACTTT | |
| | AAATCTTTTA GAAAATAATA TAGAAGCATG | |
| | CCATCAAGAC TTCAGTGTAG AGAAAAATTT | |
| | CTTATGACTC AAAGTCCTAA CCACAAAGAA | |
| | AAGATTGTTA ATTAGATTGC ATGAATATTA | |
| | AGACTTATTT TTAAAATTAA AAAACCATTA | |
| | AGAAAAGTCA GGCCATAGAA TGACAGAAAA | |
| | TATTTGCAAC ACCCCAGTAA AGAGAATTGT | |
| | AATATGCAGA TTATAAAAAG AAGTCTTACA | |
| | AATCAGTAAA AAATAAAACT AGACAAAAAT | |
| | TTGAACAGAT GAAAGAGAAA CTCTAAATAA | |
| | TCATTACACA TGAGAAACTC AATCTCAGAA | |
| | ATCAGAGAAC TATCATTGCA TATACACTAA | |
| | ATTAGAGAAA TATTAAAAGG CTAAGTAACA | |
| | TCTGTGGCTT AATTAAGGCG CGCCCCTAGG | |
| | GGCCGGCCTT AATTAAATCA AGCTTATCGA | |
| | TACCGTCGAA CCTCGAGGGG GGGCATCACT | |
| | CCGCCCTAAA ACCTACGTCA CCCGCCCCGT | |
| | TCCCACGCCC CGCGCCACGT CACAAACTCC | |
| | ACCCCCTCAT TATCATATTG GCTTCAATCC | |
| | AAAATAAGGT ATATTATTGA TGATGTTTAA | |
| | ACTACGGCCC GGTACCCAGC TTTTGTTCCC | |
| | TTTAGTGAGG GTTAATTTCG AGCTTGGCGT | |
| | AATCATGGTC ATAGCTGTTT CCTGTGTGAA | |
| | ATTGTTATCC GCTCACAATT CCACACAACA | |
| | TACGAGCCGG AAGCATAAAG TGTAAAGCCT | |
| | GGGGTGCCTA ATGAGTGAGC TAACTCACAT | |
| | TAATTGCGTT GCGCTCACTG CCCGCTTTCC | |
| | AGTCGGGAAA CCTGTCGTGC CAGCTGCATT | |
| | AATGAATCGG CCAACGCGCG GGGAGAGGCG | |
| | GTTTGCGTAT TGGGCGCTCT TCCGCTTCCT | |
| | CGCTCACTGA CTCGCTGCGC TCGGTCGTTC | |
| | GGCTGCGGCG AGCGGTATCA GCTCACTCAA | |
| | AGGCGGTAAT ACGGTTATCC ACAGAATCAG | |
| | GGGATAACGC AGGAAAGAAC ATGTGAGCAA | |
| | AAGGCCAGCA AAAGGCCAGG AACCGTAAAA | |
| | AGGCCGCGTT GCTGGCGTTT TTCCATAGGC | |
| | TCCGCCCCCC TGACGAGCAT CACAAAAATC | |
| | GACGCTCAAG TCAGAGGTGG CGAAACCCGA | |
| | CAGGACTATA AAGATACCAG GCGTTTCCCC | |
| | CTGGAAGCTC CCTCGTGCGC TCTCCTGTTC | |
| | CGACCCTGCC GCTTACCGGA TACCTGTCCG | |
| | CCTTTCTCCC TTCGGGAAGC GTGGCGCTTT | |
| | CTCATAGCTC ACGCTGTAGG TATCTCAGTT | |
| | CGGTGTAGGT CGTTCGCTCC AAGCTGGGCT | |
| | GTGTGCACGA ACCCCCCGTT CAGCCCGACC | |
| | GCTGCGCCTT ATCCGGTAAC TATCGTCTTG | |
| | AGTCCAACCC GGTAAGACAC GACTTATCGC | |
| | CACTGGCAGC AGCCACTGGT AACAGGATTA | |
| | GCAGAGCGAG GTATGTAGGC GGTGCTACAG | |
| | AGTTCTTGAA GTGGTGGCCT AACTACGGCT | |
| | ACACTAGAAG GACAGTATTT GGTATCTGCG | |
| | CTCTGCTGAA GCCAGTTACC TTCGGAAAAA | |
| | GAGTTGGTAG CTCTTGATCC GGCAAACAAA | |
| | CCACCGCTGG TAGCGGTGGT TTTTTTGTTT | |
| | GCAAGCAGCA GATTACGCGC AGAAAAAAAG | |
| | GATCTCAAGA AGATCCTTTG ATCTTTTCTA | |
| | CGGGGTCTGA CGCTCAGTGG AACGAAAACT | |
| | CACGTTAAGG GATTTTGGTC ATGAGATTAT | |
| | CAAAAAGGAT CTTCACCTAG ATCCTTTTAA | |
| | ATTAAAAATG AAGTTTTAAA TCAATCTAAA | |
| | GTATATATGA GTAAACTTGG TCTGACAGTT | |
| | ACCAATGCTT AATCAGTGAG GCACCTATCT | |
| | CAGCGATCTG TCTATTTCGT TCATCCATAG | |
| | TTGCCTGACT CCCCGTCGTG TAGATAACTA | |
| | CGATACGGGA GGGCTTACCA TCTGGCCCCA | |
| | GTGCTGCAAT GATACCGCGA GACCCACGCT | |
| | CACCGGCTCC AGATTTATCA GCAATAAACC | |
| | AGCCAGCCGG AAGGGCCGAG CGCAGAAGTG | |
| | GTCCTGCAAC TTTATCCGCC TCCATCCAGT | |
| | CTATTAATTG TTGCCGGGAA GCTAGAGTAA | |
| | GTAGTTCGCC AGTTAATAGT TTGCGCAACG | |
| | TTGTTGCCAT TGCTACAGGC ATCGTGGTGT | |
| | CACGCTCGTC GTTTGGTATG GCTTCATTCA | |
| | GCTCCGGTTC CCAACGATCA AGGCGAGTTA | |
| | CATGATCCCC CATGTTGTGC AAAAAAGCGG | |
| | TTAGCTCCTT CGGTCCTCCG ATCGTTGTCA | |
| | GAAGTAAGTT GGCCGCAGTG TTATCACTCA | |
| | TGGTTATGGC AGCACTGCAT AATTCTCTTA | |
| | CTGTCATGCC ATCCGTAAGA TGCTTTTCTG | |
| | TGACTGGTGA GTACTCAACC AAGTCATTCT | |
| | GAGAATAGTG TATGCGGCGA CCGAGTTGCT | |
| | CTTGCCCGGC GTCAATACGG GATAATACCG | |
| | CGCCACATAG CAGAACTTTA AAAGTGCTCA | |
| | TCATTGGAAA ACGTTCTTCG GGGCGAAAAC | |
| | TCTCAAGGAT CTTACCGCTG TTGAGATCCA | |
| | GTTCGATGTA ACCCACTCGT GCACCCAACT | |
| | GATCTTCAGC ATCTTTTACT TTCACCAGCG | |
| | TTTCTGGGTG AGCAAAAACA GGAAGGCAAA | |
| | ATGCCGCAAA AAAGGGAATA AGGGCGACAC | |
| | GGAAATGTTG AATACTCATA CTCTTCCTTT | |
| | TTCAATATTA TTGAAGCATT TATCAGGGTT | |
| | ATTGTCTCAT GAGCGGATAC ATATTTGAAT | |
| | GTATTTAGAA AAATAAACAA ATAGGGGTTC | |
| | CGCGCACATT TCCCCGAAAA GTGCGACGCG | |
| | GACGCGCGTA ATACGACTCA CTATAGGGCG | |
| | AATTGGAGCT CCACTACGTA GTTTAAA | |
| Human EPO | ATGGGGGTGC ACGAATGTCC TGCCTGGCTG | 19 |
| | TGGCTTCTCC TGTCCCTGCT GTCGCTCCCT | |
| | CTGGGCCTCC CAGTCCTGGG CGCCCCACCA | |
| | CGCCTCATCT GTGACAGCCG AGTCCTGGAG | |
| | AGGTACCTCT TGGAGGCCAA GGAGGCCGAG | |
| | AATATCACGA CGGGCTGTGC TGAACACTGC | |
| | AGCTTGAATG AGAATATCAC TGTCCCAGAC | |
| | ACCAAAGTTA ATTTCTATGC CTGGAAGAGG | |
| | ATGGAGGTCG GGCAGCAGGC CGTAGAAGTC | |
| | TGGCAGGGCC TGGCCCTGCT GTCGGAAGCT | |
| | GTCCTGCGGG GCCAGGCCCT GTTGGTCAAC | |
| | TCTTCCCAGC CGTGGGAGCC CCTGCAGCTG | |
| | CATGTGGATA AAGCCGTCAG TGGCCTTCGC | |
| | AGCCTCACCA CTCTGCTTCG GGCTCTGGGA | |
| | GCCCAGAAGG AAGCCATCTC CCCTCCAGAT | |
| | GCGGCCTCAG CTGCTCCACT CCGAACAATC | |
| | ACTGCTGACA CTTTCCGCAA ACTCTTCCGA | |
| | GTCTACTCCA ATTTCCTCCG GGGAAAGCTG | |
| | AAGCTGTACA CAGGGGAGGC CTGCAGGACA | |
| | GGGGACAGAT GA | |
| Optimized | ATGGGCGTGC ACGAGTGCCC CGCCTGGCTG | 20 |
| sequence of | TGGCTGCTGC TGTCCCTGCT GTCTCTGCCC | |
| human EPO | CTGGGCCTGC CTGTGCTGGG AGCCCCTCCC | |
| | CGGCTGATCT GCGACAGCCG GGTGCTGGAA | |
| | AGATACCTGC TGGAAGCCAA AGAGGCCGAG | |
| | AACATCACCA CCGGCTGCGC CGAGCACTGC | |
| | AGCCTGAACG AGAATATCAC CGTGCCCGAC | |
| | ACCAAGGTGA ACTTCTACGC CTGGAAGCGG | |
| | ATGGAAGTGG GCCAGCAGGC CGTGGAAGTG | |
| | TGGCAGGGCC TGGCCCTGCT GTCCGAGGCC | |
| | GTGCTGAGAG GGCAGGCCCT GCTGGTGAAC | |
| | AGCAGCCAGC CCTGGGAGCC TCTGCAGCTG | |
| | CACGTGGACA AGGCCGTGAG CGGCCTGCGG | |
| | AGCCTGACCA CCCTGCTGAG GGCCCTGGGC | |
| | GCCCAGAAAG AGGCCATCAG CCCCCCTGAT | |
| | GCCGCCTCTG CCGCCCCTCT GCGGACCATC | |
| | ACCGCCGACA CCTTCCGGAA GCTGTTCCGG | |
| | GTGTACAGCA ACTTCCTGCG GGGCAAGCTG | |
| | AAGCTGTACA CCGGCGAGGC CTGCCGGACC | |
| | GGCGATCGCT GA | |

### EXAMPLES

### Example 1. Characterization of Microorgan (MO) Viability in the Rat CNS with Different Washing Conditions (Implantation Studies #2 and #3)

Experiments were performed to determine optimal conditions for microorgan (MO) viability following implantation in the CNS. For these studies, surgical implantation of MOs was done in the cisterna magna (also known as the cerebellomedullary cistern). The cisterna magna was chosen as an implantation site as MOs implanted there would be expected to allow direct delivery of a secreted recombinant protein to the cerebrospinal fluid (CSF), thus efficiently delivering the molecule to the CNS. Initial experiments were done on untransfected MOs to determine optimal conditions prior to use of transduced MOs.

Rat MOs were harvested, segmented, and then cryopreserved for later use as follows. Male Lewis rats (approximately 13 weeks of age) were used to prepare MOs. To generate 25 MOs, four rats were sacrificed by CO₂ anesthesia.

Skin was shaved with a shaving machine and the dorsal site was disinfected using the following steps. First, the skin was scrubbed using Septal Scrub. Second, the procedure area plus margins was disinfected using Chlorhexidine, using circular motions starting in the center and moving towards the edge. The area was then wiped with sterile alcohol pads, moving from the center to the edge. Third, the area was scrubbed with Polydine, incubated for 10 minutes, and then Polydine was wiped away with sterile alcohol pads moving from the center to the edges. Four, the area was scrubbed again with chlorhexidine and then allowed to dry.

From the disinfected skin, MOs were prepared. Skin was cut from the dorsal pelvis up to the middle back forming a ˜8×7 cm section and attached to a plastic folio, stratum cornea (SC) facing down, using a sterile office stapler. The plastic folio was connected to the harvest platform. Using a scalpel, the skin was cut to match the width of an 80 mm dermatome. The dermatome was adjusted to maximum depth (1 mm, 17 adjustable points-0.055 mm each) and the connective tissue was separated from the skin.

The remaining skin was cut with a scalpel to approximately 30 mm width and underwent another harvesting with a 25 mm dermatome in order to extract the dermal tissue. The extracted dermal tissue was transferred immediately to a 10 cm Petri dish containing saline.

The extracted dermal tissue was then attached to a plastic folio with a 25 mm² grid using a sterile office stapler. Then, using a multi-scalpel with 1.8 mm spacers, the dermis tissue was cut lengthwise such that the tissue was aligned to the grid and that the cut of the tissue was between the 25 mm lines. Using a 75 mm dermatome blade, the edges of the MO aligned to the 25 mm lines were cut to achieve a series of 25 mm-long MOs. The MOs were transferred immediately to 10 cm Petri dish with production media. The MOs were washed 3 times with production media.

MOs were then segmented to generate 2 mm MOs. An empty petri plate was placed on top of millimeter grid paper. One 1.8 mm×25 mm MO was transferred to the petri plate and aligned along the grid. Using a scalpel, the MO was cut every 2 mm to obtain approximately 12 MOs at the size of 1.8 mm×2 mm. The segmented MOs were transferred to a 24-well plate (SARSTEDT Cat #80.1836.500 for Suspension Cells) with a single MO in well in 1 ml of production media and incubated in a 5% CO2, 32° C. incubator.

MOs were cryopreserved for later use as follows. Each MO was transferred to a Cryotube containing 200 μL of serum-free freezing cell medium (Synth-a-Freeze CTS). The Cryotubes were then transferred to a freezing container (Mr. Frosty, Thermo Scientific) and placed in a −80° C. freezer. After incubation in the freezer, Cryotubes were transferred to liquid N₂ and stored for later use.

A short thawing protocol was used to prepare the MOs from frozen Cryotubes for implantation in Implantation Studies #2 and #3. The Cryotube of MOs for the experiment was immersed in a 37° C. water bath for 1 minute with swirling. One ml of production media was added to each vial and the contents were immediately transferred into a 6-well plate containing 5 ml/well production media supplemented with 10% serum. Production media was HyClone DMEM/F-12 (Thermo scientific, Cat# SH30023.01) supplemented with 10% DCS/FBS (HyClone Defined Bovine Calf Serum supplemented, Thermo scientific, Cat. #SH30072.03) and Antibiotic-Antimycotic 1×, (Life technologies Cat. #15240-062). The MO was washed for 2 minutes with gentle swirling. Each MO was then transferred to a 24-well plate containing 1 ml production medium supplemented with 10% serum and incubated at 32° C., 5% CO2 until use. Media was exchanged every three days.

A variety of different conditions were investigated to determine optimal conditions for pre-implantation rinsing of MOs. In Implantation Study #2, MOs were thawed in fetal bovine serum (FBS) with no pre-implantation rinsing with PBS. Implantation Study #3 investigated pre-implantation rinsing protocols and substitution of Lewis rat serum for FBS (Bioreclamation: RATSRM-LEWIS-M-heat inactivated). Implantation Study #3 also included six rinses of selected MOs in PBS prior to implantation. It was hypothesized that the modifications used in preparing some MOs within Implantation Study #3 (i.e., use of Lewis rat serum and PBS rinsing prior to implantation) might decrease invasion of CD68⁺ macrophages/microglia around and into the MOs as a result of bovine proteins present. Decreased immune reaction to MOs would be predicted to lead to longer viability of the MOs.

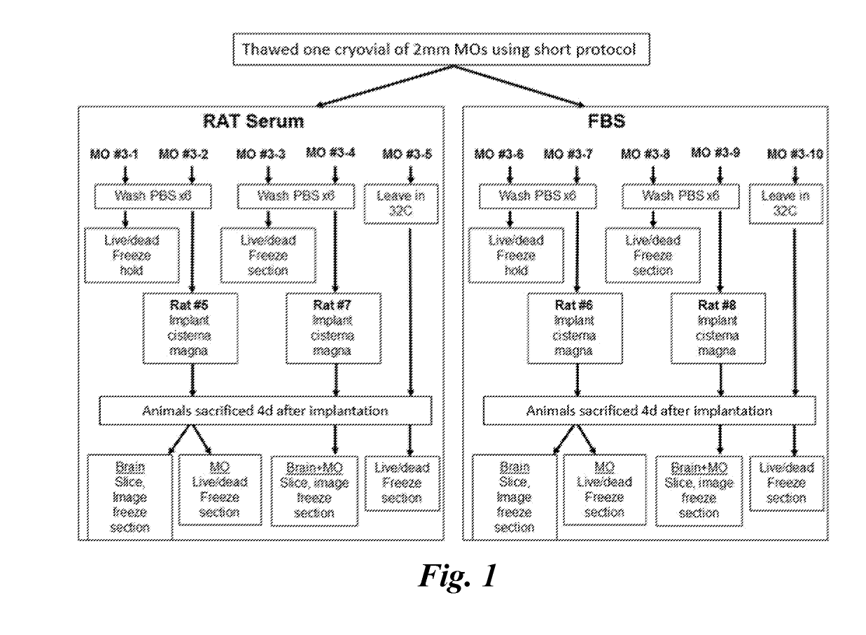

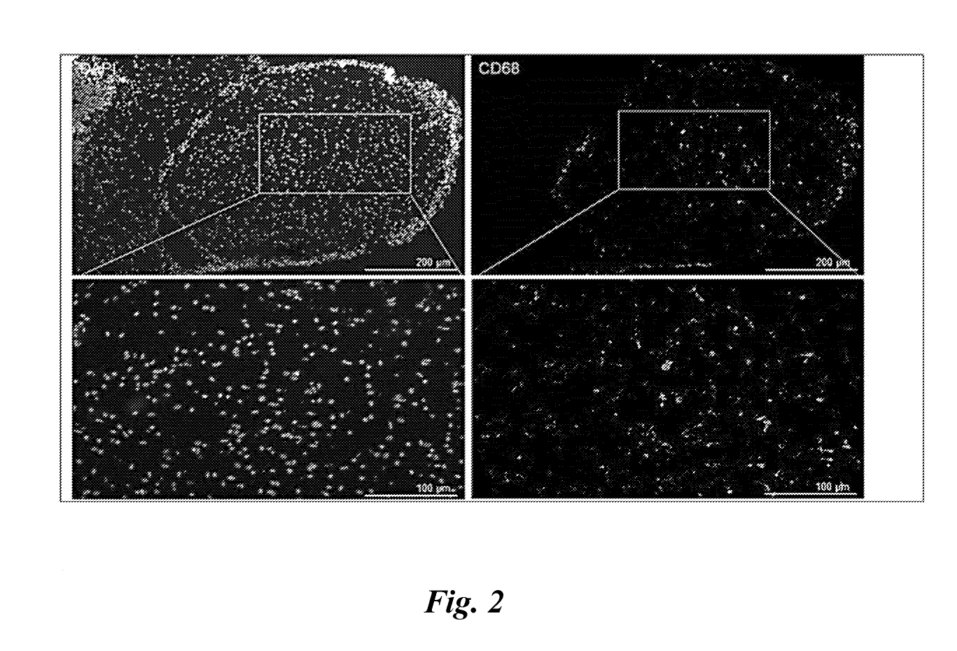

FIG. 1 outlines the conditions and study plan for Implantation Study #3. A variety of conditions were tested, including use of rat serum vs. FBS and PBS washes vs. not. Some MOs (e.g., #3-1, #3-3, #3-6, and #3-8) were analyzed for whether the MO was alive or dead (data not shown). MOs kept in-vitro were viable for the duration of the experiment. Other MOs (e.g., #3-2, #3-4, #3-5, #3-7, #3-9, and #3-10) were implanted into the cisterna magna of female Lewis rats of 15 to 20 weeks of age. The rat cisterna magna was exposed with a fine scalpel and then the MO was placed in the cisterna magna space using fine forceps. At four days after implantation of the MO, animals were sacrificed, and brains and implanted MOs were collected, sliced, and imaged as noted in FIG. 1 for histologic examination. No behavioral changes were noted in any rat during the period when the MO was implanted.

Histologic results are presented for representative MOs #2-4 (Study #2), #3-4 (Study #3), and #3-9 (Study #3) with different serum used in the production media as well as pre-implantation rinsing procedures, as described in the table below:

Summary of Process Differences for Implanted MOs
in Implantation Studies #2 and #3

| MO # | Serum | Pre-Implantation Rinsing |
|---|---|---|
| #2-4 | FBS | No rinsing |
| #3-4 | Lewis rat serum | Rinse 6x in PBS |
| #3-9 | FBS | Rinse 6x in PBS |

Slices were either stained with DAPI (at a concentration of 10 μg/ml working concentration to label the DNA of all cells in the slice) or an anti-CD68 antibody (Serotec, #MCA341R 1:500 and anti mouse secondary Vector #MP7402 to label monocytes/macrophages). Increased staining for CD68 indicates the presence of macrophages/activated microglia associated with an immune response against the MO.

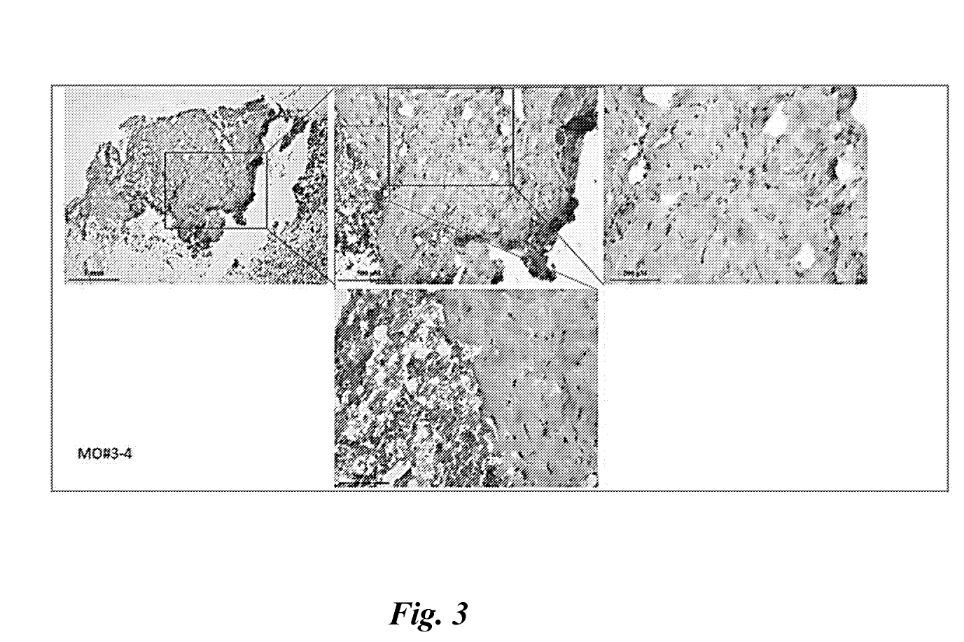

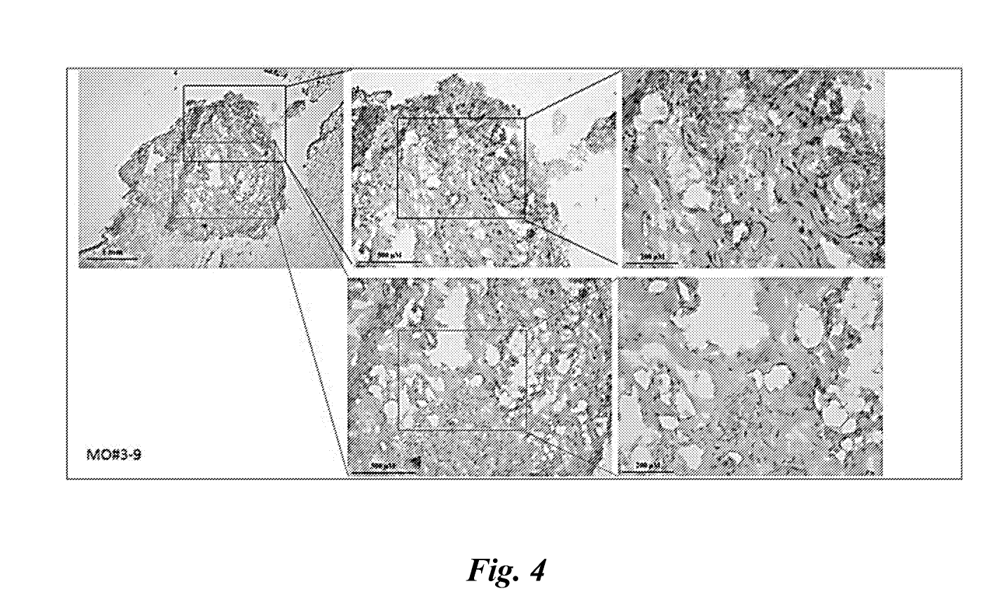

In MO#2-4 (Implantation Study #2), where the MO was thawed in FBS with no rinsing, significant numbers of CD68⁺ macrophages/activated microglia were observed surrounding the MO periphery and within the MO (FIG. 2). In MO #3-4 (Implantation Study #3), where the MO was thawed in Lewis rat serum followed by rinses six times in PBS, no CD68⁺ cells were observed surrounding or within the MO (FIG. 3) with the exception of some artifactual staining for CD68 that was found on the edges where the MO had lifted. However, in MO #3-9 (Implantation Study #3), where the MO was thawed in FBS followed by rinses six times in PBS, some CD68⁺ cells surrounded and partially invaded the MO (FIG. 4).

These data indicate that it is essential to thaw MOs in medium containing Lewis rat serum (and not FBS) to reduce the immune response of the rat to the implanted MO. Rinsing the MO in PBS prior to implantation further decreased invasion of CD68⁺ cells into MOs cultured in FBS. Thus, both use of Lewis rat serum during thawing and use of pre-implantation PBS rinses improve the outcome of MOs that are centrally implanted.

### Example 2. Characterization of Longer Implantation Times on MO Cellular Infiltration (Implantation Study #4)

Next, a study was performed to determine the impact of longer implantation times on cellular infiltration into MOs. Conditions were tested to evaluate potential reductions in the presence of macrophages or activated microglia (as measured with CD68 staining) or the presence of microglia (as measured by IBA-1) staining within the implanted MO at 14 days after implantation.

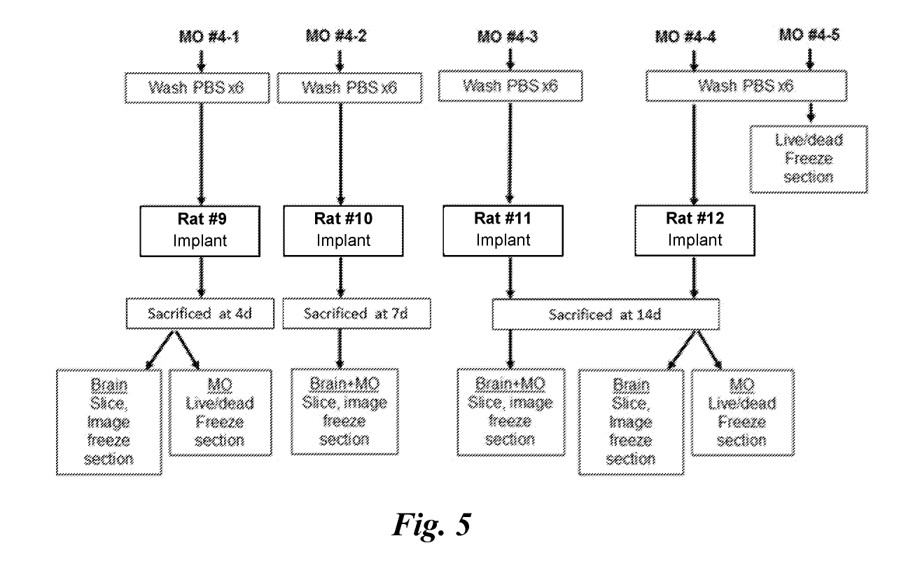

A short thaw cycle with Lewis rat serum was used in combination with six PBS rinses prior to implantation, as was shown to be optimal conditions in experiments described in Example 1. Four Lewis rats were each implanted with a single MO in the cisterna magna. One MO was harvested at 4 days post-implantation, one MO was harvested at 7 days post-implantation, and two MOs were harvested at 14 days post-implantation, as shown in FIG. 5. No behavioral changes were noted in animals while the MO was implanted. Following explanation, staining for CD68 was done as in Example 1. Staining for IBA-1 was done using goat anti IBA antibody Abcam #ab5076 1:100 and anti goat secondary Vector #MP7405.

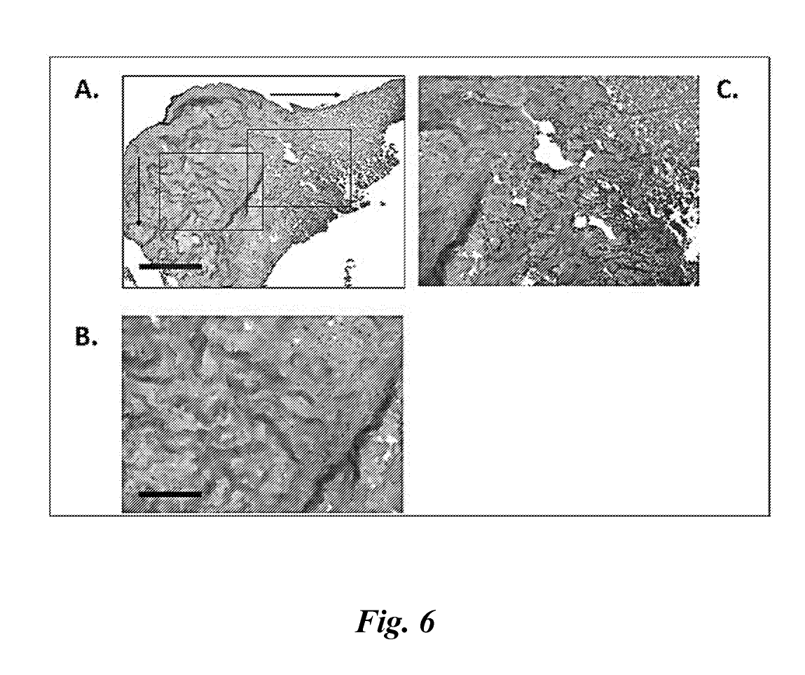

The MOs used for Implantation Study #4 were significantly larger than the MOs used in Implantation Study #3. The first MO (#4-1) did not fit into the standard-sized defect surgically created in the cisterna magna; the defect was enlarged by the neurosurgeon, which caused more than typical trauma to the cisterna magna. This MO that was harvested at 4 days post-implantation (FIG. 6A) had significantly greater cellular infiltrate on the MO periphery than previously observed (FIG. 6C) as well as a few invading cells within the MO (FIG. 6B), which may be due to additional surgical injury.

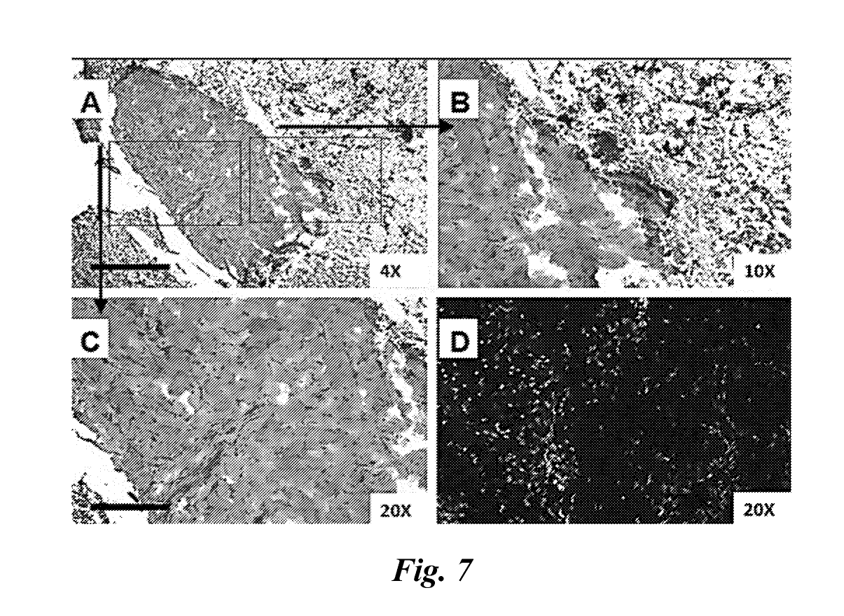

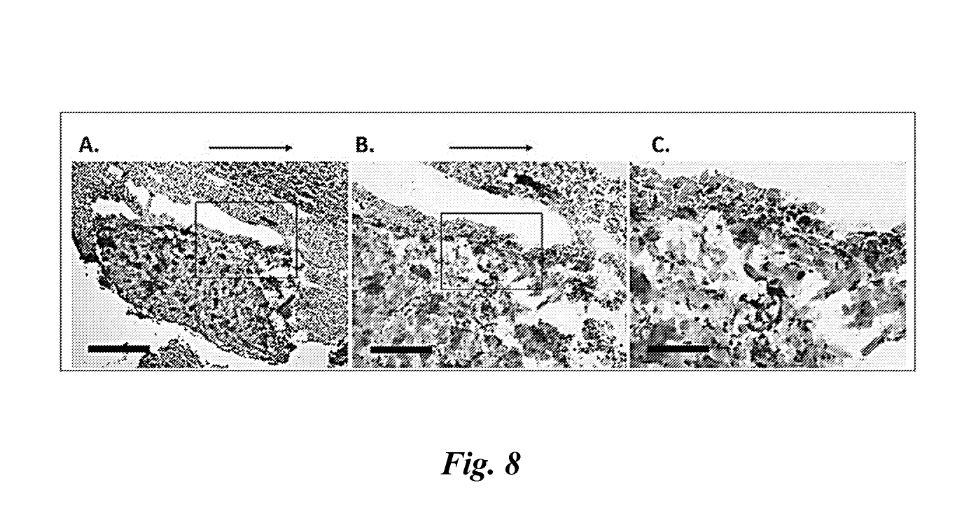

In the MO #4-2 harvested at 7 days post-implantation, hematoxylin and eosin (H&E) staining indicated that cells were uniformly dispersed throughout the MO (FIGS. 7A and 7B) without significant cellular infiltration at the periphery (FIG. 7C). FIG. 7D shows DAPI staining that indicates the presence of live cells in the MO. These data suggest suggest high viability of the implanted tissue and integration into the surrounding brain tissue. No signs of rejection or immune attack were observed. Similar results are shown in FIGS. 8A-8C, which show invading macrophages or activated microglia (CD68⁺ cells) were observed on the periphery of the MO, but not within the MO.

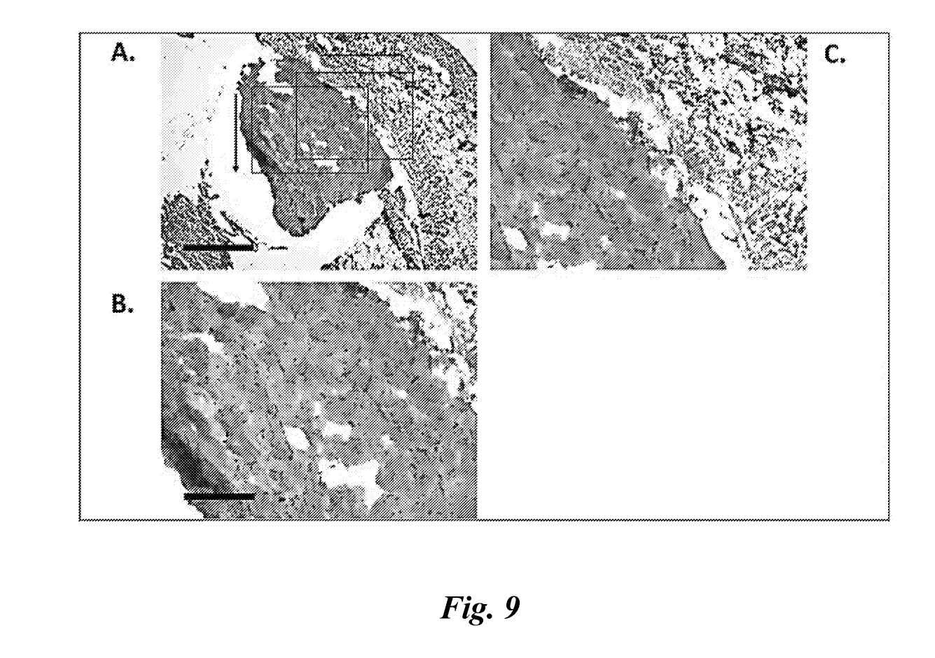

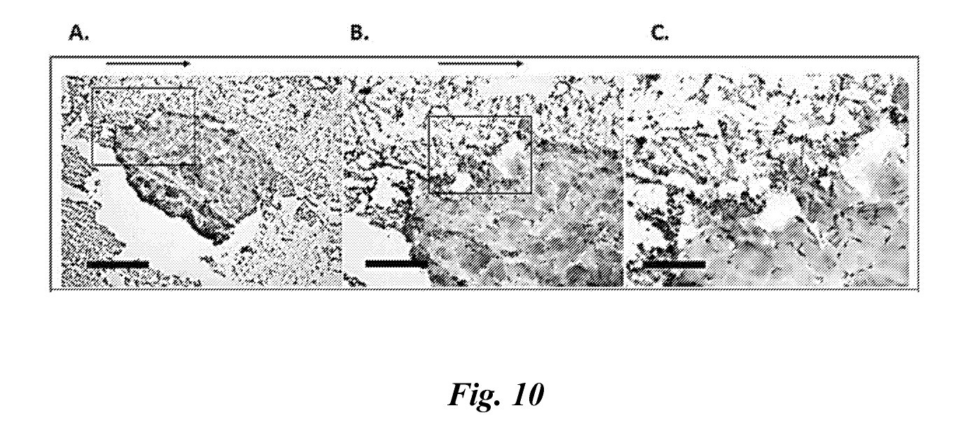

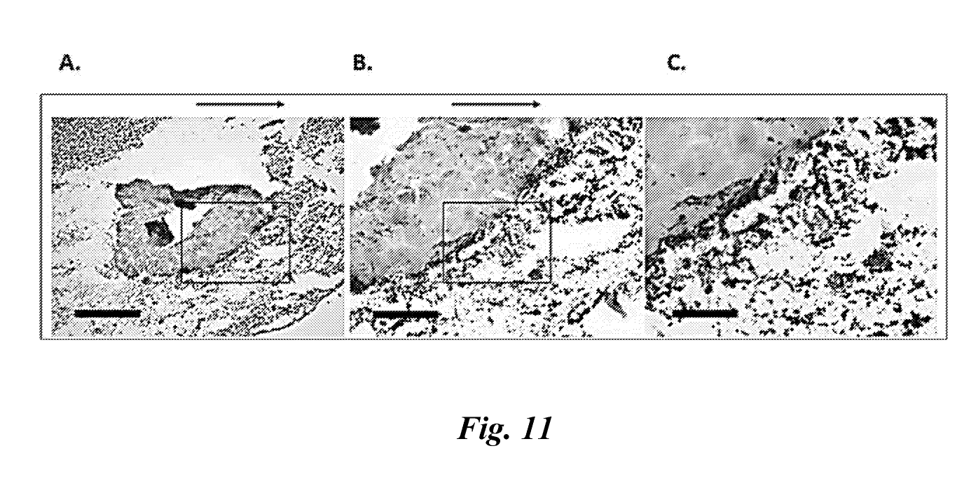

In the MO #4-3 harvested at 14 days post-implantation (FIG. 9A), H&E staining indicated that cells were again uniformly dispersed throughout the MO with fewer cells than observed at 7 days post-implantation (FIG. 9B) and relatively few invading cells at the MO periphery (FIG. 9C). Macrophages or activated microglia (CD68+) and microglia (IBA-1) were observed on the periphery but not within the MO implanted for 14 days. (FIGS. 10A-C and 11A-C, respectively).

These data indicate that autologous MOs, harvested from a donor rat and implanted in a recipient rat of the same in-bred strain, implanted for up to 14 days in Lewis rats retained viable cells and had limited infiltration by immune cells of the CNS.

### Example 3. Characterization of Erythropoietin-Secreting TARGT (TARGT_{EPOs}) in the Rat CNS (Implantation Study #5)

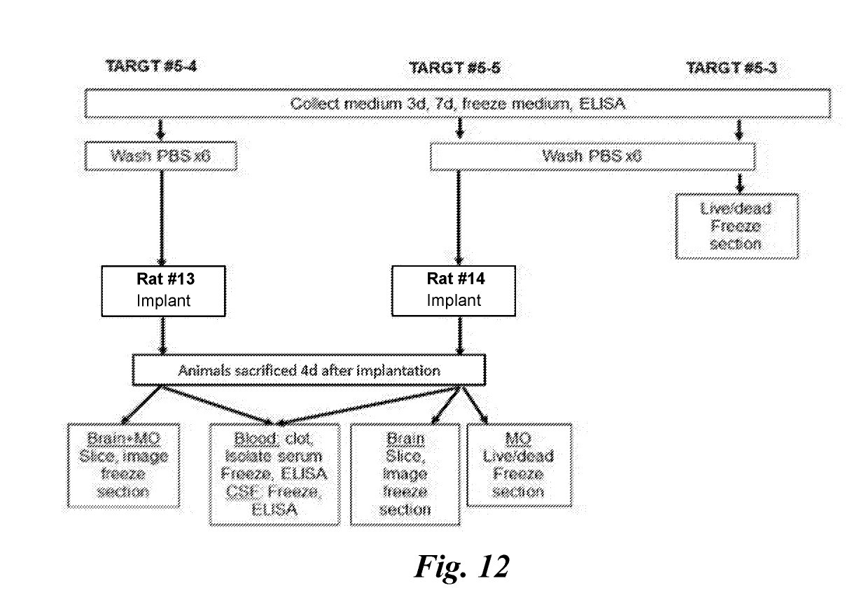

Based on the successful implantation and viability of MOs in the rat cisterna magna, further experiments were done to assess human EPO levels in the CSF and peripheral blood in rats following implantation of TARGT_{EPOs} expressing human EPO. The experimental design of this study (Implantation Study #5) is shown in FIG. 12.

TARGT_{EPOs} were generated by transduction of segmented MOs (prepared as described in Example 1) with the HDΔ28E4-MAR-EF1a-optHumanEPO-1 construct (SEQ ID No: 21) that contains an expression cassette containing the sequence of human EPO. Viral vector was diluted in production media to obtain a final concentration of 1.5×10¹⁰, as outlined in the following representative experimental calculation to generate transduction medium:

| | | | Titer | | μl/ | Total | Total |
|---|---|---|---|---|---|---|---|
| Construct name | Abbreviation | Lot# | (vp/ml) | Final conc | TARGT | vector | medium |
| HDΔ28E4-MAR- | HDÅd-MAR- | MED- | 3.85 × | 1.5 × 10\hat{ }10 | 3.95 | 98.7 | 6250 |
| EF1a-opt | EF1a-opt- | EPO- | 10\hat{ }12 | | | | |
| humanEPO-1 | hEPO | 11 | | | | | |

To perform the transduction, production media was removed from each MO well and 250 μl of transduction medium containing viral vector was added to each well. The plates were placed for 4 hours on a shaker set to 300 rpm inside an incubator (32° C., 5% CO₂) followed by overnight incubation with no shaking.

After the overnight incubation, the transduction medium was removed from the plate using a pipettor, and 2 ml of fresh production medium was added (first wash). Then, 3 ml of production medium was added to wells of a new 6-well plate, and the TARGT_{EPOs} were transferred into the wells of the new plate (second wash). The 3 ml of media was removed from each 6 well plate and fresh 3 ml media was added per well (third wash). This step was repeated another 3 times for a total of 6 washes.

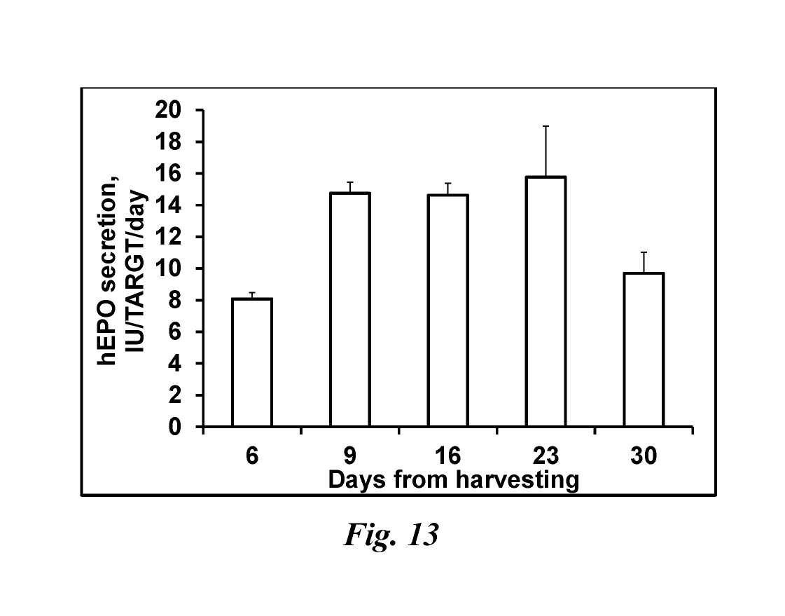

Follow transduction and washing, one set of TARGT_{EPOs} were used for in vitro validation of hEPO secretion. FIG. 13 shows the in vitro performance of 2×1 mm rat TARGT_{EPOs}, with secretion of approximately 10 IU EPO/TARGT/day. This in vitro secretion was maintained for up to 30 days post-harvesting. These results suggest that rat TARGT_{EPOs} are capable of secreting large enough amounts of human EPO such that human EPO could be measured by an ELISA for human EPO following TARGT_{EPO} implantation into the rat CNS.

Following transduction and washing, additional TARGT_{EPOs} were cryopreserved as described in Example 1.

A long thaw cycle with Lewis rat serum was used in combination with six PBS rinses prior to implantation to allow for maximum tissue viability of the TARGT_{EPOs} following thawing. The Cryotube containing an MO was immersed in a 37° C. water bath for one minute with swirling. One ml production media containing 50% serum was added into each vial, and the contents were immediately transferred into 6-well plates containing 5 ml/well production media supplemented with 50% serum. The MOs were washed for 2 minutes with gentle swirling. Each MO was transferred to a 24-well plate containing 1 ml production media supplemented with 50% serum and incubated in 32° C., 5% CO₂ for 4 hours. Each MO was then transferred to a well of a new 24-well plate containing 1 ml production media supplemented with 20% serum and incubated in 32° C., 5% CO₂ for 20 hours. Finally, each MO was transferred to a well of a new 24-well plate containing 1 ml production medium supplemented with 10% serum and incubated in 32° C., 5% CO₂ until use. Media was exchanged every three days.

Two Lewis rats were implanted with one TARGT_{EPO} each in the cisterna magna. The TARGT_{EPOs} were then harvested at 4 days post-implantation with no behavioral changes noted while the TARGT_{EPO} was implanted. On the day of explantation, CSF was first collected by lumbar puncture. Subsequently, the animal was sacrificed; blood was collected through cardiac puncture and the brain and TARGT_{EPO} was harvested.

Information on the findings during explantation and the collected CSF and peripheral blood are presented in Table 1.

TABLE 1
Explantation observations

| | | | Peripheral |
|---|---|---|---|
| TARGT | TARGT Explantation | CSF | Blood |
| #5-4 | TARGT anchored to soft tissue outside | 85 μL | 2 mL |
| Rat #13 | brain. TARGT pulled out upon skull | collected. | |
| | removal | Clear | |
| #5-5 | Liquid drop (CSF?) on closed head | 40 μL | 2 mL |
| Rat #14 | incision, observed prior to | collected. | |
| | explantation. TARGT also anchored to | Tinged | |
| | soft tissue; broke connection prior to | with red | |
| | skull removal so TARGT remained in | | |
| | brain. | | |

As described in Table 1, during the 4 days of implantation in Implantation Study #5, the protruding end of the TARGT_{EPO} anchored itself to the soft tissue used to close the wound in both rat (#13 and #14) implanted with TARGT_{EPO} in the cisterna magna. TARGT_{EPO} attachment to soft tissue is ideal for delivery of nutrients and oxygen, but care is required at explantation from the CNS to avoid disturbing the implanted TARGT_{EPO}. During the first explantation, the TARGT_{EPO} (#5-4) was pulled out of the implantation site in rat #13 when the skull was removed. Thus, TARGT_{EPO} #5-4 was used for the viability testing, and the brain and TARGT_{EPO} were processed separately for histology. In the second explantation, the TARGT_{EPO} (#5-5) was again attached to the soft tissue in rat #14 but was successfully detached prior to skull removal. Thus, TARGT_{EPO} #5-5 and its surrounding brain were processed together for histology.

As also shown in Table 1, there was variability in the collection of CSF prior to animal sacrifice. In rat #13 (implanted with TARGT_{EPO} #5-4), approximately 85 μL of CSF were collected in multiple lumbar punctures. However, only 40 μL of CSF was collected from rat #14 (implanted with TARGT_{EPO} #5-5). Prior to lumbar puncture, a drop of fluid was observed on the closed incision at the original implantation site in rat #14. This fluid was likely CSF, which leaked out of the implantation site. Only a small volume of slightly red CSF was collected by lumbar puncture; the color was not removed by centrifugation. Because of the issues with CSF collection, the EPO level could not be accurately and reproducibly measured from rat #14 (implanted with TARGT_{EPO} #5-5), and data on EPO levels will only be presented for rat #13 (implanted with TARGT_{EPO} #5-4).

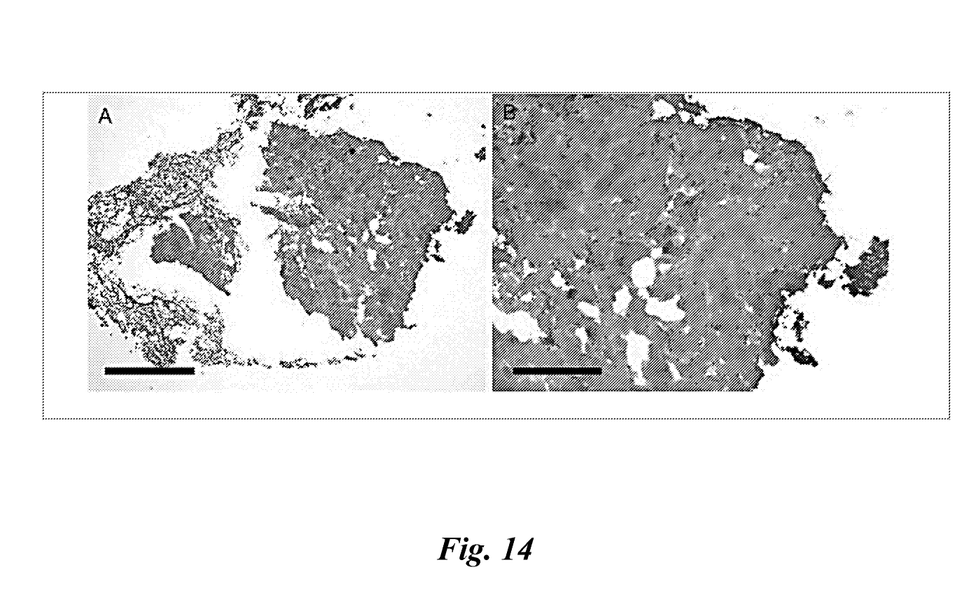

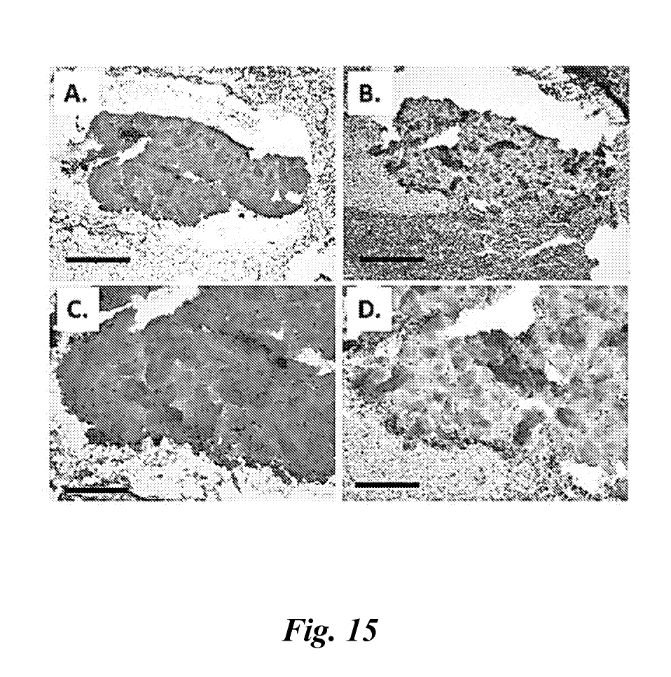

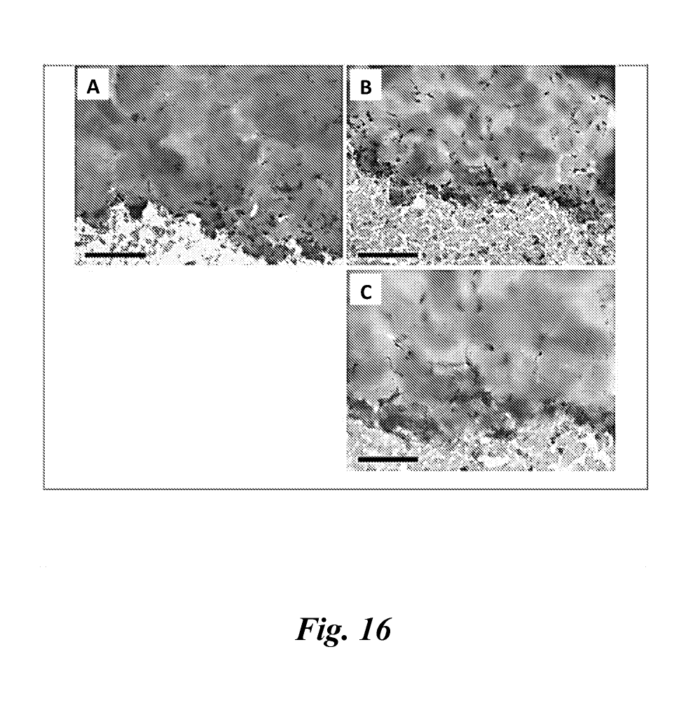

H&E staining of TARGT_{EPO} #5-4 showed little cellular infiltration (FIGS. 14A-B). H&E staining and CD68 labelling of TARGT_{EPO} #5-5 were also performed. At 4 days post-implantation, cells were uniformly dispersed throughout TARGT_{EPO} #5-5 based on H&E staining (FIGS. 15A and 15C). As in previous implantations, macrophages or activated microglia (CD68⁺) were observed on the periphery, while very few CD68⁺ cells were found within the TARGT_{EPO} matrix (FIGS. 15B and 15D). FIGS. 16A-C show higher magnification data from TARGT_{EPO} #5-5, confirming uniform number of cells throughout the TARGT without significant cellular infiltration from the periphery.

### A. EPO Concentration

Experiments were done to determine EPO secretion following implantation of TARGT_{EPOs} using a human EPO ELISA kit (Quantikine IVD, Human Epo Immunoassay, Cat # DEP00, R&D Systems, Inc.) following manufacturer protocols. At baseline, no human EPO was detected in the blood or cerebrospinal fluid (CSF) of rats implanted with MOs that had not been transduced to express EPO (data not shown). Thus, the presence of human EPO in the blood or CSF of rats implanted with a TARGT_{EPO} would indicate successful expression and secretion of human EPO by the TARGT, as native rat EPO does not cross-react with human EPO in this ELISA.

EPO concentrations for TARGT_{EPO} #5-4 were measured by ELISA in the medium during TARGT_{EPO} thawing and also in the CSF and peripheral blood serum at 4 days after implantation, sampled prior to animal sacrifice. As shown in Table 2, TARGT_{EPO} #5-4 expressed EPO at Day 3 and Day 7 post-thaw in vitro. TARGT_{EPO} #5-4 also successfully expressed and secreted human EPO when implanted in the cisterna magna, as human EPO was detected in the CSF. Significantly lower levels of human EPO were measured in the serum of the peripheral blood, indicating some leakage of EPO from the CNS into the peripheral blood. The much higher levels of EPO in the CSF compared to peripheral blood indicates the central delivery of EPO by the TARGT_{EPO} implanted in the cisterna magna. Values in Table 2 represent levels of human EPO, which is distinguished from the native rat EPO. A summary of data from the in vivo study of TARGT_{EPOs} is presented in Table 3.

TABLE 2
EPO concentrations for TARGT #5-4 in medium during
thawing and in the CSF and serum of peripheral blood
after 4 days implantation, as determined by ELISA

| | EPO concentration, | EPO concentration, |
|---|---|---|
| Condition | sample 1 (mIU/ml) | sample 2 (mIU/ml) |
| #5-4 in vitro medium, day 3 | 7652 | 6920 |
| post-thaw | | |
| #5-4 in vitro medium, day 7 | 11351 | 11644 |
| post-thaw | | |
| #5-4 CSF, day 4 post- | 1622 | |
| implantation | | |
| #5-4 serum of peripheral | 24.81 | 18.48 |
| blood, day 4 post-implantation | | |

TABLE 3
In vivo secretion levels of
TARGT_{EPOs} implanted in rat cisterna magna

| | Ave hEPO | | Total | | |
|---|---|---|---|---|---|
| TARGT | conc. | Vol. | hEPO | Collection | hEPO Rate |
| condition | (mIU/mL) | (mL) | (mIU) | time (hr) | (mIU/hr) |
| In-vitro, 3 | 7,286 | 1 | 7,286 | 72 | 101 |
| days post | | | | | |
| thaw | | | | | |
| In-vitro, 7 | 11,497 | 1 | 11,497 | 96 | 120 |
| days post | | | | | |
| thaw | | | | | |
| CSF - post | 1,622 (1.622 | 0.09 | 146 | | 146* |
| implantation | mIU/μL) | | | | |
| Serum post- | 21 | 12 | 252 | | |
| implantation | | | | | |

*assuming all volume of CSF is being produced and replaced every hour.

Results indicate high levels of secretion of EPO by the TARGT_{EPO} in culture at 3 and 7 days after thawing with secretion levels of around 120 mIU/hr, showing that secretion of EPO by the TARGT_{EPO} was retained after freezing and thawing of the MO.

Thus, implantation of a TARGT_{EPO} in the cisterna magna can lead to successfully secretion of EPO into the CSF, as evidenced by the fact that human EPO was present only in the rat that had been implanted with TARGT_{EPO} and not in those implanted with nontransduced MOs. These secretion results measured in vivo in rat CSF post-TARGT implantation into the cisterna magna suggest high recovery of the implanted dose, since rat CSF is produced and replaced every hour. Lower levels of hEPO were also detected in rat serum.

Thus, this study with central implantation of TARGT_{EPO} yielded promising results. At Day 4 post-implantation, the host response to the TARGT_{EPO} implanted in the cisterna magna was minimal and was similar to that of the response to non-transduced MOs (as presented in Examples 1 and 2). Human EPO was detected in the CSF as well as the serum of the peripheral blood at Day 4 post-implantation, indicating successful delivery of EPO within the CNS by TARGT_{EPO}.

### Example 4. Generation of Pig TARGT-Adalimumab and Central Implantation of TARGT-Adalimumab in Pigs

Pigs are a model to study larger TARGTs than those that can be studied in a rodent. Pigs are also a closer model to the human CNS in terms of head size, brain size, CSF volume, ventricular system size, space of the brain, and serum volume. The pig dermis is also more similar to human dermis than rodent dermis for investigating dermal micro-organs. In addition, the implantation tools and techniques used in pig studies are more relevant to humans. Thus, dosing studies in pigs of micro-organ implantation in the CNS is highly relevant to human usage of micro-organs.

Dermal MOs were prepared from pigs using the following procedures. Pigs used for harvesting of dermal MOs were shaved using a shaving blade, disinfected, and scrubbed with Septal Scrub prior to the pig being placed on the operating room bed. Once the surgeon was scrubbed, the procedure area plus margins were disinfected with chlorhexidine using circular movements starting in the center and moving to the edges. The area was then wiped using sterile drapes, moving from the center to the edge. The scrubbing of the area was then repeated using Polydine. After that, the unsterile area was covered with sterile drapes to define the sterile procedure area. The Polydine was incubated for 10 minutes, before it was wiped off using sterile drapes, moving from the center to the edges. Once in the operating room, the pig was anesthetized and mechanically ventilated.

MOs were then harvested in operation room using the NOUVAG chuck driller; NOUVAG motor set at 7000 rpm, chuck driller, Dermavac 3.5 mm equipped with 14 G needle, and back vacuum containing 2 ml of saline. After harvesting, the MOs were vacuumed out from the distal end of the needle to the attached syringe or flashed out from the proximal end of the needle. The MO's were divided into 50 ml tubes each with 15 ml of production medium with 10% pig serum [DMEM F-12 (ADCF) with phenol red (HyClone cat N# SH30023) supplemented with 10% porcine serum (B.I cat#:04-006-1A) and antibiotic stock of penicillin 10,000 units, streptomycin 10 mg and 25 μg, and amphotericin B/ml (SIGMA cat-A5955)]. The final concentration in the media is as follows: Penicillin: 100 U/ml, Streptomycin: 100 μg/ml, and Amphotericin-B: 0.25 μg/ml. MOs were then washed three times in production media without serum inside a Petri dish. Following, these washes the MOs were incubated with 1 ml production media, in 24-well plates in 5% CO₂ incubator at 32° C. for 24 hr-72 hr.

TARGT-adalimumab were then prepared by viral transduction of the pig dermal MOs. MOs were transduced with a viral vector that encodes adalimumab to generate a TARGT-adalimumab that is a pig MO that expresses and secretes human adalimumab. The viral vector used to generate TARGT-adalimumab was HDdelta28E4-MAR-EF1a-optHumAb1-1. Information of the viral vector is as follows:

| Construct name | Lot# | Titer (vp/TARGT) |
|---|---|---|
| HDdelta28E4-MAR-EF1a-optHumAb1-1 | 10114A | 9.72 × 10E12 vp/ml |

Transduction of pig MOs was done in a similar manner to that described for rat MOs. Eight pig MOs were transduced with viral vector diluted in pig production media to a final concentration of 1.5λ10¹¹ viral particles/TARGT (130 μL/TARGT+2100 μL production media). Following preparation of viral vector in production media, 250 μL of this transduction medium was added to each well containing a TARGT. Plates with TARGTs in transduction medium were placed on a shaker place set to 300 rmp inside an incubator set to 32° C., 5% CO₂ overnight.

After incubation, the TARGT-adalimumab were washed. The transduction medium (250 μl) was removed from the plate using a pipettor, and 2 ml of fresh production medium was added (first wash). Then, 3 ml of production medium was added to wells of a new 6-well plate, and the TARGTs were transferred into the wells of the new plate (second wash). The 3 ml of media was then removed from each 6 well plate, and fresh 3 ml media is added per well (third wash). The final wash step was repeated for three more times. The TARGTs were then be transferred to a new 24-well plate with fresh 1 ml production media per well and incubated in a 5% CO₂ incubator at 32° C. Media was exchanged every day and spent media samples evaluated for secretion of antibody. These TARGT-adalimumabs were used to implant into the CNS of the same pig (i.e., autologous implantation) at 7-10 days post-harvest.

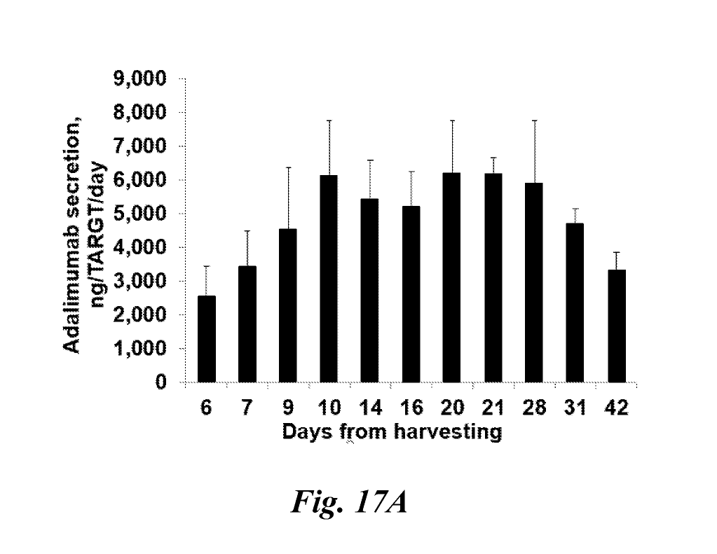

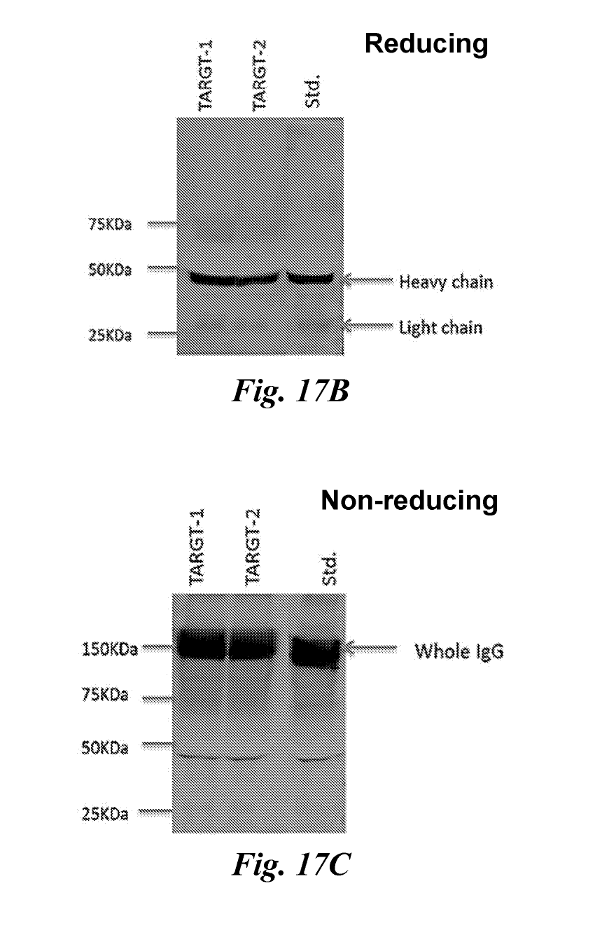

The in vitro performance of pig TARGT-adalimumabs was also assessed. FIG. 17A shows results on secretion of adalimumab by TARGT-adalimumabs over 42 days. In-vitro assessment of pig TARGT-adalimumabs indicate prolonged secretion of adalimumab at a level of micrograms per day. FIGS. 17B-C show reducing (FIG. 17B) and non-reducing (FIG. 17C) western blot analysis of adalimumab secreted in vitro by pig TARGT-adalimumabs. The western blot analysis of adalimumab secreted in-vitro by pig TARGT-adalimumabs suggests that this adalimumab has a similar size and structure to commercial adalimumab (Humira®, labeled as “std.”). Thus, in-vitro results with pig TARGT-adalimumabs suggest prolonged secretion of fully-folded, proper molecular weight adalimumab, consistent with the profile of commercially-available Humira, at levels of micrograms per day.

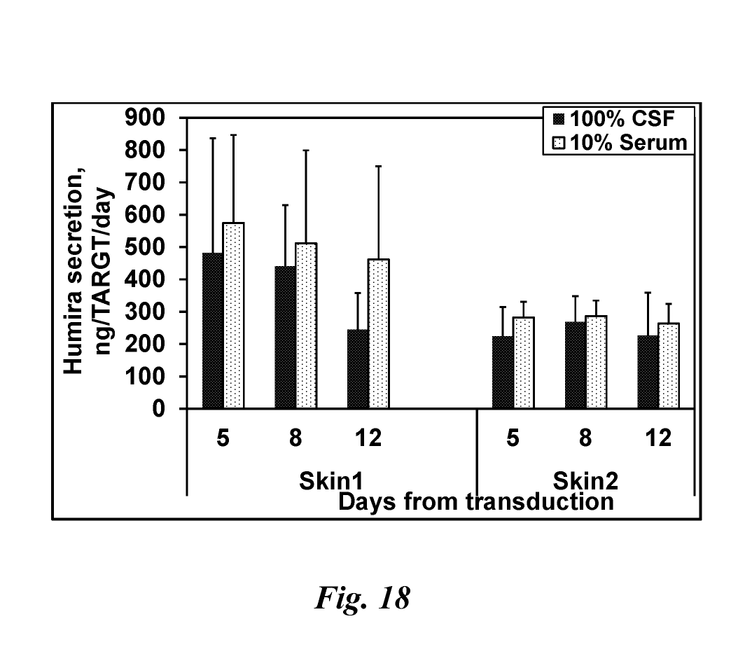

The profile of TARGT-adalimumabs maintained in vitro in 100% CSF was compared to those maintained in DMEM-F12 media supplemented with 10% serum (FIG. 18). These in-vitro results suggest that pig CSF may support TARGT-adalimumab maintenance for at least two weeks. This period of time may be enough to allow TARGT-adalimumab integration post-implantation into the CNS.

In preparation for the implantation of the TARGT-adalimumab into the CNS, a lumbar catheter was implanted to allow CSF sampling. A catheter was placed in the lower lumbar space via a standard lumbar puncture procedure. About 20 cm of catheter length was inserted. The catheter cap was replaced with a cap comprising a septum which allows drawing CSF with a needle without removing the cap (heparin lock yellow cap). This procedure allows CSF drawing from the pig while it is not anaesthetized. The catheter was fixated using sutures to the skin in two places and in addition glued to the skin with Histoacryl. Synthomycine ointment was applied at the catheter outlet and the area was covered with Tegaderm sterile adhesive bandage. This catheterization allows daily CSF sampling.

Next, sub dural implantation of TARGT-adalimumab was performed. The forehead skin was opened with a cut 5 cm above the canthal line (the line between the 2 eyes at the level of the angle between the superior and inferior eyelids). Further cutting of sub dermal layers was done till reaching the periost. The periost was separated from the bone using a spatula and the entire cut was retracted in order to expose the surgical field.

Two burr holes were made in the cranium using a craniotome with a 12 mm drill. A Kerrison tool was used to cut the excess bone and reach the dura. To allow better access with tools for the sub-dura implantation, a 3 mm cutting tool was used to mill a recess on the edge of the burr hole. A minimal cut (4-5 mm) was done in the dura mater to approach the sub-dura space, using scalpel and tweezer.

TARGT-adalimumab were then prepared for insertion into the sub-dura space. Using custom tweezers, a suture was inserted in the middle of each TARGT-adalimumab (0-6 Suture 9.3 mm needle). One TARGT-adalimumab was inserted into each approach to the sub-dura space through the cut in the dura using blunt tweezers. Therefore, each pig was implanted with two TARGT-adalimumabs.

A catheter similar to the one inserted into the lumbar space was inserted in the right burr hole following TARGT-adalimumab insertion. This catheter was first inserted through the forehead skin using a needle to reach the surgical site allowing most of the catheter to be subdermal with only a small section of it on the skin surface.

Dura cut closure was done using 0-6 suture monofilament W8305 Prolene. Cutanplast was inserted into the burr holes. The head catheter was sutured, stapled, and glued (using Histoacryl) to the skin. The surgical cut was sutured in the subcutaneous and skin layers using Vicryl and Prolene sutures, respectively.

Results obtained post implantation suggests no observed pig's behavioral change.

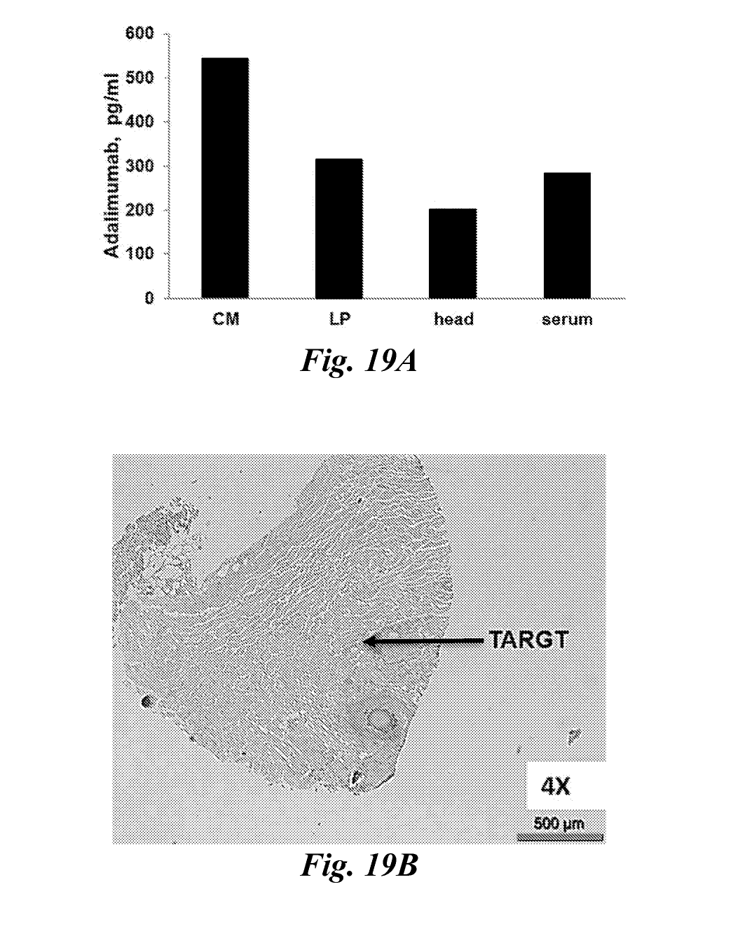

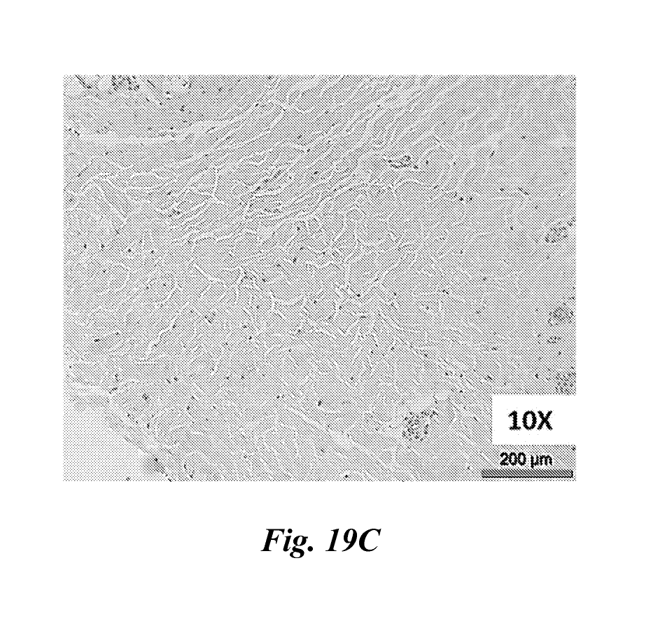

At 7 days after implantation, adalimumab was measured in CSF samples taken from the implantation area (cisterna magna), the lumbar space, the sub-dura, and serum. Results in FIG. 19A show adalimumab levels of hundreds of pg per ml were achieved in vivo, with distribution in CSF sampled from pig cisterna magna (CM), sub-dura (head), and lumbar (LP). Adalimumab was also measurable in the serum.

One-week post-implantation TARGTs were excised out of the pig brain. Histopathology analysis of excised TARGT-adalimumabs using H&E staining in FIGS. 19B (4× magnification) and 19C (10× magnification) show tissue viability and no sign of inflammation. The collagen within the TARGT-adalimumab appeared normal, and several blood vessels were identified within the TARGT-adalimumab (suggesting initial integration into the dura).

These data in pigs support the ability to TARGT-adalimumabs to secrete adalimumab in vivo in a pig model. Adalimumab was detected is CSF sampled from the cisterna magna, sub-dura, and lumbar regions at seven-days post-implantation. Furthermore, histopathology analysis of excised TARGT-adalimumabs at one-week post-implantation suggest tissue viability and no signs of inflammation or rejection. Thus, central implantation of TARGT-adalimumabs was a means for allowing secretion of adalimumab in the CNS over an extended time period.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

What is claimed is:

1. A method for treating cancer comprising implanting a micro-organ into the central nervous system (CNS), wherein the micro-organ secretes a recombinant protein, and wherein the micro-organ is maintained in the CNS, and secretes protein, for at least seven days.

2. The method of claim 1, wherein secretion of the recombinant protein is measurable in the CNS for a sustained period of time of at least one week, at least one month, at least two months, at least three months, at least four months, at least five months, at least six months, at least seven months, at least eight months, at least nine months, at least ten months, at least eleven months, or at least twelve months.

3. The method of claim 1, wherein secretion of the recombinant protein is measurable outside of the CNS for a sustained period of time of at least one week, at least one month, at least two months, at least three months, at least four months, at least five months, at least six months, at least seven months, at least eight months, at least nine months, at least ten months, at least eleven months, or at least twelve months.

4. The method of claim 1, wherein the micro-organ is implanted at the same time as a procedure for biopsy, removal, or debulking of a CNS tumor.

5. The method of any one of claims 1-4, wherein the cancer is a primary CNS tumor(s) or a tumor(s) secondary to a cancer with origins outside of the CNS.

6. The method of any one of claims 1-5, wherein the cancer is or has an astrocytoma, glioblastoma, glioma, lymphoma, medulloblastoma, or CNS lymphoma.

7. The method of any of claims 1-6, wherein the cancer in the CNS is secondary to colon, kidney, melanoma, lung, ovarian, breast, or testicular cancer.

8. The method of any of claims 1-7, wherein the protein secreted by the micro-organ is an antibody.

9. The method of claim 8, wherein the antibody is trastuzumab, anti-PD1, cetuximab, an immune check-point antibody, or rituximab.

10. The method of any of claims 1-9, further comprising administration of a biologic or non-biologic chemotherapeutic agent.

11. The method of any of claims 1-10, wherein the secretion of the recombinant protein within the CNS is monitored by measurement of levels in the cerebrospinal fluid.

12. The method of claim 11, wherein a catheter is implanted to allow periodic measurement of cerebrospinal fluid.

13. The method of any of claims 1-12, wherein the level of recombinant protein is measured via imaging of the brain and/or spinal cord.

14. The method of any of claims 1-13, wherein the level of the recombinant protein the CNS determines the timing of removal of the micro-organ(s) and the timing of subsequent implantations of additional micro-organ(s).

15. A method for treating a lysosomal storage disease comprising implanting a micro-organ into the central nervous system (CNS), wherein the micro-organ secretes a recombinant protein, and wherein the micro-organ is maintained in the CNS, and secretes protein, for at least seven days.

16. The method of claim 15, wherein secretion of the recombinant protein is measurable in the CNS for a sustained period of time of at least one week, at least one month, at least two months, at least three months, at least four months, at least five months, at least six months, at least seven months, at least eight months, at least nine months, at least ten months, at least eleven months, or at least twelve months.

17. The method of claim 15, wherein secretion of the recombinant protein is measurable outside of the CNS for a sustained period of time of at least one week, at least one month, at least two months, at least three months, at least four months, at least five months, at least six months, at least seven months, at least eight months, at least nine months, at least ten months, at least eleven months, or at least twelve months.

18. The method of any of claims 15-17, wherein the lysosomal storage disease is Hunter syndrome, Fabry disease, Infantile Batten disease (CNL1), Classic late infantile Batten disease (CNL2), Hurler syndrome, Krabbe disease, Niemann-Pick A, Niemann-Pick B, Pompe disease, Batten disease, Gaucher disease, or Tay Sachs disease.

19. The method of any of claims 15-18, wherein the recombinant protein replaces a gene product that is not expressed or that is misexpressed due to a genetic mutation.

20. The method of any of claims 15-19, wherein the secretion of the recombinant protein by the micro-organ is monitored by measurement of levels in the cerebrospinal fluid.

21. The method of claim 20, wherein a catheter is implanted to allow periodic measurement of cerebrospinal fluid.

22. The method of claim 20, wherein expression of the recombinant protein is measurable in the cerebrospinal fluid for a sustained period of time of at least one week, at least one month, at least two months, at least three months, at least four months, at least five months, at least six months, at least seven months, at least eight months, at least nine months, at least ten months, at least eleven months, or at least twelve months.

23. The method of any of claims 15-22, wherein levels of the recombinant protein in the cerebrospinal fluid determine the timing of removal of the genetically modified micro-organ(s) or the timing of subsequent implantations of genetically modified micro-organ(s).

24. The method of any of claims 15-24, wherein the protein is an antibody.

25. A method of preparing a micro-organ for implantation into the CNS comprising i) removing a micro-organ of non-CNS tissue; ii) maintaining the micro-organ in vitro for 1 to 7 days; iii) transducing the micro-organ with a viral vector comprising a therapeutic protein; and iv) freezing the transduced micro-organ.

26. The method of claim 25, wherein steps iii) and iv) are reversed so that the micro-organ is frozen prior to transduction.

27. A method of implanting a microorgan into the CNS, comprising making an incision in the dura and inserting a micro-organ, wherein the micro-organ secretes a recombinant protein into the sub-dural space and outside of the sub-dural space.

28. The method of claim 27, wherein the micro-organ is inserted into the spine, cisterna magna, ventricular system space of the brain, brain convexity, or brain parenchyma.


SEQUENCE LISTING (20 sequences)

SEQ ID NO: 1 - DNA, 744 bp, Artificial sequence
  Optimized light chain sequence from adalimumab contained in TNF1 construct
gacatccaaatgacacagagtccttcctccttgtcagctagtgttggagaccgcgttactatcacatgcagggcgtcacaaggcatcaggaattacttggcgtggtaccagcagaagcctggaaaagccccaaaactgctgatatacgcagccagcacacttcaatcaggcgtgccctctaggttctctggctccggttccggaaccgacttcacactcaccatatcctcactgcaacctgaagacgtggccacatactattgtcagcgctataatagggcaccctacacttttggccaagggacgaaagtggaaataaaaaggacagtggcagctccgtccgtttttatcttccctccatccgatgagcagcttaagtctgggactgcttccgtagtgtgtttgctgaataatttttatccccgagaagcaaaggttcagtggaaggtcgataatgccctgcagagtggcaatagtcaggagtccgtaaccgagcaggactctaaggactccacctattccctgagttccaccttgaccctttccaaggccgactatgagaagcacaaagtatacgcctgcgaggtaactcaccagggattgagctccccagtgacaaagtcatttaatcggggcgagtgcctgtccaaggccgactacgaaaagcacaaagtgtacgcctgtgaagtcacccatcagggcctgtcatctccagtcacgaagtcattcaatcgaggggagtgc

SEQ ID NO: 2 - DNA, 744 bp, Artificial sequence
  Optimized light chain sequence from adalimumab contained in TNF construct
gacatccagatgacgcagtccccaagctcactgtccgcctctgtaggtgaccgggtaactatcacctgcagagcatcccagggcatccgcaattacctggcctggtatcagcagaaacctggcaaggccccaaaactcctcatctacgcagcatccacccttcagagtggcgtaccaagccgattctccggaagcggtagtggaaccgactttaccctcacaatctcaagtctgcagcctgaagatgtcgctacatattattgccagagatacaatagggccccatacacctttgggcagggcacgaaagtggaaattaagcgcacagttgcggcaccaagtgtgtttattttcccgcccagcgatgaacagctgaaatccggcacggccagcgttgtatgcttgctgaataacttttaccctagagaggccaaggtccaatggaaggttgacaacgcactgcagtccggcaacagtcaagagagcgtcactgaacaagattccaaggacagtacatactcactcagctccacactgacactctccaaggccgactacgagaagcataaggtctacgcttgcgaggtaacgcatcagggcctttctagcccagttaccaaaagtttcaatcgaggcgaatgcctgtcaaaagcagactacgagaaacacaaggtttacgcctgtgaagtgacacaccagggcttgagctcccctgtgacaaaatcttttaataggggagagtgt

SEQ ID NO: 3 - DNA, 1353 bp, Artificial sequence
  Optimized heavy chain sequence from adalimumab contained in TNF construct
gaagtgcagcttgtggagtctggcggtggcctcgtgcagccaggccggagcctgcggctgagctgtgcagccagcgggttcaccttcgatgattatgctatgcactgggttcgccaggcccccggaaagggcctggagtgggtctcagctatcacatggaattccggacacatcgactacgccgacagcgtggaggggcgctttaccatttcaagggacaacgctaaaaacagcctgtaccttcagatgaactccctgcgggcggaagacacagcggtgtactactgtgccaaggtgagctacctgtccacagcatcctcattggactattggggccaaggcacgctggttaccgtttccagcgcaagcacaaagggacctagtgtgttcccgttggccccttcaagcaaatccacgagtggaggcaccgctgcactgggctgccttgtaaaggactacttcccggagccagtgactgtgtcatggaacagtggcgccctgacaagcggagtccacacttttcctgcggtcctccagtcctccgggctttacagcctgagtagtgtggttaccgtcccctcatcctccctgggtacccagacctacatttgtaatgtgaaccataagccaagcaatacaaaggtggataaaaaggtggagccaaaaagctgcgataaaacacatacttgccctccttgcccagcgcccgagttgctcggcggcccttccgtatttctttttccaccgaaaccgaaggatacactgatgatctctcggacccctgaggtcacttgtgtggtggttgacgtttcacacgaggacccagaagtgaagtttaattggtacgtggatggggttgaggtgcacaatgctaaaaccaagccgcgcgaggagcaatataactctacctatcgagtggtgagcgtgctcaccgtactccatcaggactggctgaacgggaaggagtacaagtgcaaggtttcaaacaaggctctccctgccccaatagagaagaccataagtaaagccaagggacagcctcgcgagccacaggtctatactctgcctcctagtagggacgagctcaccaagaaccaggtaagcctcacctgcttggtcaagggcttttatccatccgacatcgccgtggaatgggagagcaacggacagcctgaaaacaactacaaaactaccccacccgttcttgattcagatgggagcttttttctgtacagcaagttgaccgtcgataaatcccgatggcagcagggaaatgttttctcttgctcagtgatgcatgaagcgctgcacaaccactatacacagaagagccttagcttgtctccaggaaaa

SEQ ID NO: 4 - DNA, 1353 bp, Artificial sequence
  Optimized heavy chain sequence from adalimumab contained in TNF construct
gaagtgcagttggtcgagtccggtggagggctggtccagcctggcagaagtctccggctgagttgcgcagccagcggattcaccttcgacgattacgccatgcactgggtgcggcaggccccgggcaagggccttgaatgggtgtctgcgatcacatggaattccggacatattgattacgccgacagcgtggagggccgattcaccatcagtagggataatgctaagaactccctgtacctgcagatgaatagtctgagggctgaagacacagccgtgtactattgcgcaaaagtcagctacctctccactgcttctagtctggactactggggtcaggggacgctggtgacggtttcttccgcatccactaaaggtcctagcgttttccccctcgccccctcttctaagagcacctccggaggaactgcagcccttggatgcttggttaaagattactttcccgaacccgtaaccgtaagctggaacagtggcgccctgacttcaggggtacacacctttccggccgtgctgcagagcagcgggctctatagccttagctcagtcgtgacggtcccatcctctagtcttggtactcaaacctacatctgcaatgtgaatcacaagccttctaacacaaaagttgataagaaagtagaacccaagagctgtgataagacacatacttgtcctccctgtccggcccccgaattgcttggggggccgagtgtcttcctcttccctccaaaacccaaggacactctcatgatttcaaggacccctgaagtgacttgtgtggtagttgacgtgagccacgaggaccctgaagtgaagttcaattggtatgtggatggcgttgaggtgcataatgcaaagacaaagccacgcgaggagcagtacaattccacctatagggtggtatccgtgctgaccgtgttgcatcaggactggctcaatgggaaagagtataaatgtaaggtgtccaataaggccctgcccgctcccattgaaaaaacaatttcaaaggctaagggccaaccccgcgaaccacaagtctacacactcccccctagtagagatgagctgacaaaaaatcaggtgtctctcacatgtctggtaaaaggcttctatccttcagatattgctgtggaatgggaatcaaatgggcagccagagaataactacaaaacgacacccccagtccttgatagtgacgggtccttcttcctctactctaaactcaccgtggacaagagtagatggcaacagggcaatgtgttctcctgtagcgtcatgcatgaagcactgcacaatcattatactcagaagagcttgtcccttagtccaggaaaa

SEQ ID NO: 5 - DNA, 136 bp, Artificial sequence
  Heavy chain signal sequence containing intron
ggatggagctgtatcatcctcttcttggtagcaacagctacaggtaaggggttaacagtagcaggcttgaggtctggacatatatatgggtgacaatgacatccactttgcctttctctccacaggcgcgcactcc

SEQ ID NO: 6 - DNA, 423 bp, Artificial sequence
  CMV enhancer
gagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtg

SEQ ID NO: 7 - DNA, 224 bp, Artificial sequence
  EF1-alpha promoter
actagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattc

SEQ ID NO: 8 - DNA, 427 bp, Artificial sequence
  CpG-free MAR from human beta-globin gene
ttaattaaaattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtaccagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaaggctagcctgcag

SEQ ID NO: 9 - DNA, 808 bp, Homo sapiens
  misc_feature: MAR 5′ region from human IFN-beta gene
agtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcaagacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcttaattaa

SEQ ID NO: 10 - DNA, 231 bp, Artificial sequence
  SV40 polyadenylation signal
ccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattc

SEQ ID NO: 11 - DNA, 12 bp, Artificial sequence
  Furin sequence
cgggcaaaacgg

SEQ ID NO: 12 - DNA, 72 bp, Artificial sequence
  2A sequence
gctcccgttaaacagacgctgaatttcgatctcctgaagttggccggagacgtcgaatcaaaccccggccca

SEQ ID NO: 13 - DNA, 23 bp, Artificial sequence
  IRES sequence
atgataatatggccacaaccatg

SEQ ID NO: 14 - DNA, 2540 bp, Artificial sequence
  Sequence of the TNF1 construct
gttggtgtacagtagtagcaagcttgcatgcctgcaggtcgactctagactgccatgggatggagctgtatcatcctcttcttggtagcaacagctacaggtaaggggttaacagtagcaggcttgaggtctggacatatatatgggtgacaatgacatccactttgcctttctctccacaggcgcgcactccgacatccaaatgacacagagtccttcctccttgtcagctagtgttggagaccgcgttacttcacatgcagggcgtcacaaggcatcaggaattacttggcgtggtaccagcagaagcctggaaaagccccaaaactgctgatatacgcagccagcacacttcaatcaggcgtgccctctaggttctctggctccggttccggaaccgacttcacactcaccatatcctcactgcaacctgaagacgtggccacatactattgtcagcgctataatagggcaccctacacttttggccaagggacgaaagtggaaataaaaaggacagtggcagctccgtccgtttttatcttccctccatccgatgagcagcttaagtctgggactgcttccgtagtgtgtttgctgaataatttttatccccgagaagcaaaggttcagtggaaggtcgataatgccctgcagagtggcaatagtcaggagtccgtaaccgagcaggactctaaggactccacctattccctgagttccaccttgaccctttccaaggccgactatgagaagcacaaagtatacgcctgcgaggtaactcaccagggattgagctccccagtgacaaagtcatttaatcggggcgagtgcctgtccaaggccgactacgaaaagcacaaagtgtacgcctgtgaagtcacccatcagggcctgtcatctccagtcacgaagtcattcaatcgaggggagtgccgggcaaaacgggctcccgttaaacagacgctgaatttcgatctcctgaagttggccggagacgtcgaatcaaaccccggcccaggatggagctgtatcatcctcttcttggtagcaacagctacaggtaaggggttaacagtagcaggcttgaggtctggacatatatatgggtgacaatgacatccactttgcctttctctccacaggcgcgcactccgaagtgcagcttgtggagtctggcggtggcctcgtgcagccaggccggagcctgcggctgagctgtgcagccagcgggttcaccttcgatgattatgctatgcactgggttcgccaggcccccggaaagggcctggagtgggtctcagctatcacatggaattccggacacatcgactacgccgacagcgtggaggggcgctttaccatttcaagggacaacgctaaaaacagcctgtaccttcagatgaactccctgcgggcggaagacacagcggtgtactactgtgccaaggtgagctacctgtccacagcatcctcattggactattggggccaaggcacgctggttaccgtttccagcgcaagcacaaagggacctagtgtgttcccgttggccccttcaagcaaatccacgagtggaggcaccgctgcactgggctgccttgtaaaggactacttcccggagccagtgactgtgtcatggaacagtggcgccctgacaagcggagtccacacttttcctgcggtcctccagtcctccgggctttacagcctgagtagtgtggttaccgtcccctcatcctccctgggtacccagacctacatttgtaatgtgaaccataagccaagcaatacaaaggtggataaaaaggtggagccaaaaagctgcgataaaacacatacttgccctccttgcccagcgcccgagttgctcggcggcccttccgtatttctttttccaccgaaaccgaaggatacactgatgatctctcggacccctgaggtcacttgtgtggtggttgacgtttcacacgaggacccagaagtgaagtttaattggtacgtggatggggttgaggtgcacaatgctaaaaccaagccgcgcgaggagcaatataactctacctatcgagtggtgagcgtgctcaccgtactccatcaggactggctgaacgggaaggagtacaagtgcaaggtttcaaacaaggctctccctgccccaatagagaagaccataagtaaagccaagggacagcctcgcgagccacaggtctatactctgcctcctagtagggacgagctcaccaagaaccaggtaagcctcacctgcttggtcaagggcttttatccatccgacatcgccgtggaatgggagagcaacggacagcctgaaaacaactacaaaactaccccacccgttcttgattcagatgggagcttttttctgtacagcaagttgaccgtcgataaatcccgatggcagcagggaaatgttttctcttgctcagtgatgcatgaagcgctgcacaaccactatacacagaagagccttagcttgtctccaggaaaatgaggatccccgggagatatcctaggcttgg

SEQ ID NO: 15 - DNA, 2486 bp, Artificial sequence
  Sequence of the TNF3 construct
gttggtgtacagtagtagcaagcttgcatgcctgcaggtcgactctagactgccatgggatggagctgtatcatcctcttcttggtagcaacagctacaggtaaggggttaacagtagcaggcttgaggtctggacatatatatgggtgacaatgacatccactttgcctttctctccacaggcgcgcactccgacatccagatgacgcagtccccaagctcactgtccgcctctgtaggtgaccgggtaactatcacctgcagagcatcccagggcatccgcaattacctggcctggtatcagcagaaacctggcaaggccccaaaactcctcatctacgcagcatccacccttcagagtggcgtaccaagccgattctccggaagcggtagtggaaccgactttaccctcacaatctcaagtctgcagcctgaagatgtcgctacatattattgccagagatacaatagggccccatacacctttgggcagggcacgaaagtggaaattaagcgcacagttgcggcaccaagtgtgtttattttcccgcccagcgatgaacagctgaaatccggcacggccagcgttgtatgcttgctgaataacttttaccctagagaggccaaggtccaatggaaggttgacaacgcactgcagtccggcaacagtcaagagagcgtcactgaacaagattccaaggacagtacatactcactcagctccacactgacactctccaaggccgactacgagaagcataaggtctacgcttgcgaggtaacgcatcagggcctttctagcccagttaccaaaagtttcaatcgaggcgaatgcctgtcaaaagcagactacgagaaacacaaggtttacgcctgtgaagtgacacaccagggcttgagctcccctgtgacaaaatcttttaataggggagagtgttgaatgataatatggccacaaccatgatgggatggagctgtatcatcctcttcttggtagcaacagctacaggtaaggggttaacagtagcaggcttgaggtctggacatatatatgggtgacaatgacatccactttgcctttctctccacaggcgcgcactccgaagtgcagttggtcgagtccggtggagggctggtccagcctggcagaagtctccggctgagttgcgcagccagcggattcaccttcgacgattacgccatgcactgggtgcggcaggccccgggcaagggccttgaatgggtgtctgcgatcacatggaattccggacatattgattacgccgacagcgtggagggccgattcaccatcagtagggataatgctaagaactccctgtacctgcagatgaatagtctgagggctgaagacacagccgtgtactattgcgcaaaagtcagctacctctccactgcttctagtctggactactggggtcaggggacgctggtgacggtttcttccgcatccactaaaggtcctagcgttttccccctcgccccctcttctaagagcacctccggaggaactgcagcccttggatgcttggttaaagattactttcccgaacccgtaaccgtaagctggaacagtggcgccctgacttcaggggtacacacctttccggccgtgctgcagagcagcgggctctatagccttagctcagtcgtgacggtcccatcctctagtcttggtactcaaacctacatctgcaatgtgaatcacaagccttctaacacaaaagttgataagaaagtagaacccaagagctgtgataagacacatacttgtcctccctgtccggcccccgaattgcttggggggccgagtgtcttcctcttccctccaaaacccaaggacactctcatgatttcaaggacccctgaagtgacttgtgtggtagttgacgtgagccacgaggaccctgaagtgaagttcaattggtatgtggatggcgttgaggtgcataatgcaaagacaaagccacgcgaggagcagtacaattccacctatagggtggtatccgtgctgaccgtgttgcatcaggactggctcaatgggaaagagtataaatgtaaggtgtccaataaggccctgcccgctcccattgaaaaaacaatttcaaaggctaagggccaaccccgcgaaccacaagtctacacactcccccctagtagagatgagctgacaaaaaatcaggtgtctctcacatgtctggtaaaaggcttctatccttcagatattgctgtggaatgggaatcaaatgggcagccagagaataactacaaaacgacacccccagtccttgatagtgacgggtccttcttcctctactctaaactcaccgtggacaagagtagatggcaacagggcaatgtgttctcctgtagcgtcatgcatgaagcactgcacaatcattatactcagaagagcttgtcccttagtccaggaaaatgaggatccccgggagatatcctaggcttgg

SEQ ID NO: 16 - DNA, 8101 bp, Artificial sequence
  Sequence of pAd-MAR-EF1a-opt hTNF1
ggccgattcattaatgcaggggccgctgcggccatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggaggcgcgccttaattaaaattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtaccagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaaggctagcctgcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcatctgccttctccctcctgtgagtttggtaagtcactgactgtctatgcctgggaaagggtgggcaggaggtggggcagtgcaggaaaagtggcactgtgaaccctgcagccctagacaattgtactaaccttcttctctttcctctcctgacaggttggtgtacagtagtagcaagcttgcatgcctgcaggtcgactctagactgccatgggatggagctgtatcatcctcttcttggtagcaacagctacaggtaaggggttaacagtagcaggcttgaggtctggacatatatatgggtgacaatgacatccactttgcctttctctccacaggcgcgcactccgacatccaaatgacacagagtccttcctccttgtcagctagtgttggagaccgcgttactatcacatgcagggcgtcacaaggcatcaggaattacttggcgtggtaccagcagaagcctggaaaagccccaaaactgctgatatacgcagccagcacacttcaatcaggcgtgccctctaggttctctggctccggttccggaaccgacttcacactcaccatatcctcactgcaacctgaagacgtggccacatactattgtcagcgctataatagggcaccctacacttttggccaagggacgaaagtggaaataaaaaggacagtggcagctccgtccgtttttatcttccctccatccgatgagcagcttaagtctgggactgcttccgtagtgtgtttgctgaataatttttatccccgagaagcaaaggttcagtggaaggtcgataatgccctgcagagtggcaatagtcaggagtccgtaaccgagcaggactctaaggactccacctattccctgagttccaccttgaccctttccaaggccgactatgagaagcacaaagtatacgcctgcgaggtaactcaccagggattgagctccccagtgacaaagtcatttaatcggggcgagtgcctgtccaaggccgactacgaaaagcacaaagtgtacgcctgtgaagtcacccatcagggcctgtcatctccagtcacgaagtcattcaatcgaggggagtgccgggcaaaacgggctcccgttaaacagacgctgaatttcgatctcctgaagttggccggagacgtcgaatcaaaccccggcccaggatggagctgtatcatcctcttcttggtagcaacagctacaggtaaggggttaacagtagcaggcttgaggtctggacatatatatgggtgacaatgacatccactttgcctttctctccacaggcgcgcactccgaagtgcagcttgtggagtctggcggtggcctcgtgcagccaggccggagcctgcggctgagctgtgcagccagcgggttcaccttcgatgattatgctatgcactgggttcgccaggcccccggaaagggcctggagtgggtctcagctatcacatggaattccggacacatcgactacgccgacagcgtggaggggcgctttaccatttcaagggacaacgctaaaaacagcctgtaccttcagatgaactccctgcgggcggaagacacagcggtgtactactgtgccaaggtgagctacctgtccacagcatcctcattggactattggggccaaggcacgctggttaccgtttccagcgcaagcacaaagggacctagtgtgttcccgttggccccttcaagcaaatccacgagtggaggcaccgctgcactgggctgccttgtaaaggactacttcccggagccagtgactgtgtcatggaacagtggcgccctgacaagcggagtccacacttttcctgcggtcctccagtcctccgggctttacagcctgagtagtgtggttaccgtcccctcatcctccctgggtacccagacctacatttgtaatgtgaaccataagccaagcaatacaaaggtggataaaaaggtggagccaaaaagctgcgataaaacacatacttgccctccttgcccagcgcccgagttgctcggcggcccttccgtatttctttttccaccgaaaccgaaggatacactgatgatctctcggacccctgaggtcacttgtgtggtggttgacgtttcacacgaggacccagaagtgaagtttaattggtacgtggatggggttgaggtgcacaatgctaaaaccaagccgcgcgaggagcaatataactctacctatcgagtggtgagcgtgctcaccgtactccatcaggactggctgaacgggaaggagtacaagtgcaaggtttcaaacaaggctctccctgccccaatagagaagaccataagtaaagccaagggacagcctcgcgagccacaggtctatactctgcctcctagtagggacgagctcaccaagaaccaggtaagcctcacctgcttggtcaagggcttttatccatccgacatcgccgtggaatgggagagcaacggacagcctgaaaacaactacaaaactaccccacccgttcttgattcagatgggagcttttttctgtacagcaagttgaccgtcgataaatcccgatggcagcagggaaatgttttctcttgctcagtgatgcatgaagcgctgcacaaccactatacacagaagagccttagcttgtctccaggaaaatgaggatccccgggagatatcctaggcttggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattcagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcaagacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcttaattaaggcgcgccgggcccctacgtcacccgccccgttcccacgccccgcgccacgtcacaaactccaccccctcattatcatattggcttcaatccaaaataaggtatattattgatgatggccgcagcggccctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggggccgctgcggccatcatcaataatataccttattttggattgaagccaata

SEQ ID NO: 17 - DNA, 8042 bp, Artificial sequence
  Sequence of pAd-MAR-EF1a-opt hTNF3
ggccgattcattaatgcaggggccgctgcggccatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggaggcgcgccttaattaaaattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtaccagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaaggctagcctgcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcatctgccttctccctcctgtgagtttggtaagtcactgactgtctatgcctgggaaagggtgggcaggaggtggggcagtgcaggaaaagtggcactgtgaaccctgcagccctagacaattgtactaaccttcttctctttcctctcctgacaggttggtgtacagtagtagcaagcttgcatgcctgcaggtcgactctagactgccatgggatggagctgtatcatcctcttcttggtagcaacagctacaggtaaggggttaacagtagcaggcttgaggtctggacatatatatgggtgacaatgacatccactttgcctttctctccacaggcgcgcactccgacatccagatgacgcagtccccaagctcactgtccgcctctgtaggtgaccgggtaactatcacctgcagagcatcccagggcatccgcaattacctggcctggtatcagcagaaacctggcaaggccccaaaactcctcatctacgcagcatccacccttcagagtggcgtaccaagccgattctccggaagcggtagtggaaccgactttaccctcacaatctcaagtctgcagcctgaagatgtcgctacatattattgccagagatacaatagggccccatacacctttgggcagggcacgaaagtggaaattaagcgcacagttgcggcaccaagtgtgtttattttcccgcccagcgatgaacagctgaaatcccggccagcgttgtatgcttgctgaataacttttaccctagagaggccaaggtccaatggaaggttgacaacgcactgcagtccggcaacagtcaagagagcgtcactgaacaagattccaaggacagtacatactcactcagctccacactgacactctccaaggccgactacgagaagcataaggtctacgcttgcgaggtaacgcatcagggcctttctagcccagttaccaaaagtttcaatcgaggcgaatgcctgtcaaaagcagactacgagaaacacaaggtttacgcctgtgaagtgacacaccagggcttgagctcccctgtgacaaaatcttttaataggggagagtgttgaatgataatatggccacaaccatgatgggatggagctgtatcatcctcttcttggtagcaacagctacaggtaaggggttaacagtagcaggcttgaggtctggacatatatatgggtgacaatgacatccactttgcctttctctccacaggcgcgcactccgaagtgcagttggtcgagtccggtggagggctggtccagcctggcagaagtctccggctgagttgcgcagccagcggattcaccttcgacgattacgccatgcactgggtgcggcaggccccgggcaagggccttgaatgggtgtctgcgatcacatggaattccggacatattgattacgccgacagcgtggagggccgattcaccatcagtagggataatgctaagaactccctgtacctgcagatgaatagtctgagggctgaagacacagccgtgtactattgcgcaaaagtcagctacctctccactgcttctagtctggactactggggtcaggggacgctggtgacggtttcttccgcatccactaaaggtcctagcgttttccccctcgccccctcttctaagagcacctccggaggaactgcagcccttggatgcttggttaaagattactttcccgaacccgtaaccgtaagctggaacagtggcgccctgacttcaggggtacacacctttccggccgtgctgcagagcagcgggctctatagccttagctcagtcgtgacggtcccatcctctagtcttggtactcaaacctacatctgcaatgtgaatcacaagccttctaacacaaaagttgataagaaagtagaacccaagagctgtgataagacacatacttgtcctccctgtccggcccccgaattgcttggggggccgagtgtcttcctcttccctccaaaacccaaggacactctcatgatttcaaggacccctgaagtgacttgtgtggtagttgacgtgagccacgaggaccctgaagtgaagttcaattggtatgtggatggcgttgaggtgcataatgcaaagacaaagccacgcgaggagcagtacaattccacctatagggtggtatccgtgctgaccgtgttgcatcaggactggctcaatgggaaagagtataaatgtaaggtgtccaataaggccctgcccgctcccattgaaaaaacaatttcaaaggctaagggccaaccccgcgaaccacaagtctacacactcccccctagtagagatgagctgacaaaaaatcaggtgtctctcacatgtctggtaaaaggcttctatccttcagatattgctgtggaatgggaatcaaatgggcagccagagaataactacaaaacgacacccccagtccttgatagtgacgggtccttcttcctctactctaaactcaccgtggacaagagtagatggcaacagggcaatgtgttctcctgtagcgtcatgcatgaagcactgcacaatcattatactcagaagagcttgtcccttagtccaggaaaatgaggatccccgggagatatcctaggcttggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattcagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcaagacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcttaattaaggcgcgccgggcccctacgtcacccgccccgttcccacgccccgcgccacgtcacaaactccaccccctcattatcatattggcttcaatccaaaataaggtatattattgatgatggccgcagcggccctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggggccgctgcggccatcatcaataatataccttattttggattgaagccaata

SEQ ID NO: 18 - DNA, 33057 bp, Artificial sequence
  HD-delta-28E4-MAR-EF1a-optHumanEPO-1
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttgatatcaagcttatcgataccgtaaacaagtctttaattcaagcaagactttaacaagttaaaaggagcttatgggtaggaagtagtgttatgatgtatgggcataaagggttttaatgggatagtgaaaatgtctataataatacttaaatggctgcccaatcacctacaggattgatgtaaacatggaaaaggtcaaaaacttgggtcactaaaatagatgattaatggagaggatgaggttgatagttaaatgtagataagtggtcttattctcaataaaaatgtgaacataaggcgagtttctacaaagatggacaggactcattcatgaaacagcaaaaactggacatttgttctaatctttgaagagtatgaaaaattcctattttaaaggtaaaacagtaactcacaggaaataccaacccaacataaaatcagaaacaatagtctaaagtaataaaaatcaaacgtttgcacgatcaaattatgaatgaaattcactactaaaattcacactgattttgtttcatccacagtgtcaatgttgtgatgcatttcaattgtgtgacacaggcagactgtggatcaaaagtggtttctggtgcgacttactctcttgagtatacctgcagtcccctttcttaagtgtgttaaaaaaaaagggggatttcttcaattcgccaatactctagctctccatgtgctttctaggaaacaagtgttaacccaccttatttgtcaaacctagctccaaaggacttttgactccccacaaaccgatgtagctcaagagagggtatctgtcaccagtatgtatagtgaaaaaagtatcccaagtcccaacagcaattcctaaaaggagtttatttaaaaaaccacacacacctgtaaaataagtatatatcctccaaggtgactagttttaaaaaaacagtattggctttgatgtaaagtactagtgaatatgttagaaaaatctcactgtaaccaagtgaaatgaaagcaagtatggtttgcagagattcaaagaaaatataagaaaacctactgttgccactaaaaagaatcatatattaaatatactcacacaatagctcttcagtctgataaaatctacagtcataggaatggatctatcactatttctattcagtgctttgatgtaatccagcaggtcagcaaagaatttatagccccccttgagcacacagagggctacaatgtgatggcctcccatctccttcatcacatctcgagcaagacgttcagtcctacagaaataaaatcaggaatttaatagaaagtttcatacattaaactttataacaaacacctcttagtcattaaacttccacaccaacctgggcaatatagtgagaccccatgcctgcaaaaaaaaaaaaattagccaggcatggtagcatgtacctgtagtcccagctacttgagaggtgaggtgggaaaatcactttagtgcaggatgttgaggctggagtgaactgtgattgtgccactgcactccagcctggacaatagagcaagaccttgtctcaaaaaaatgcattaaaaattttttttaaatcttccacgtatcacatcctttgccctcatgtttcataaggtaaaaaatttgataccttcaaaaaaaccaagcataccactatcataattttttttaaatgcaaataaaaacaagataccattttcacctatcagactggcaggttctgattaaatgaaattttctggataatatacaatattaagagagactgtagaaactgggccagtggctcatgcctgtaatcccagcactttgggaggctgggtaacatggcgaaccctgtttctacaaaataaaaatattagctgggagtggtggcgcacacctatagtcccagctactcaggaggctgaggtggaaggatcgcttgaacccaggaggttgagactgcagtgaactgtgatcattctgctgcactgcaccccagcctgggcaacagagaccttgtctcaaaaaaaaaaaaaaaagagacaaattgtgaagagaaaggtactctcatataacatcaggagtataaaatgattcaacttcttagaggaaaatttggcaataccaaaatattcaataaactctttccccttgacccagaaattccacttgaataaagctgaacaagtaccaaacatgtaaaagaatgtttcttctagtacagtcggtaagaacaaaatagtgtctatcaatagtggactggttaaatcagttatggtatctccataagacagaatgctatgcaacctttaaaatatattagatagctctagacacactaatattaaaagtgtccaataacatttaaaactatactcatacgttaaaatataaatgtatatatgtacttttgcatatagtatacatgcataggccagtgcttgagaagaaatgtgtacagaaggctgaaaggagagaactttagtcttcttgtttatggcctccatagttagaatattttataacacaaatattttgatattataattttaaaataaaaacacagaatagccagacatacaatgcaagcattcaataccaggtaaggtttttcactgtaattgacttaacagaaaattttcaagctagatgtgcataataataaaaatctgaccttgccttcatgtgattcagccccagtccattaccctgtttaggactgagaaatgcaagactctggctagagttccttcttccatctcccttcaatgtttactttgttctggtccctacagagtcccactataccacaactgatactaagtaattagtaaggccctcctcttttatttttaataaagaagattttagaaagcatcagttatttaataagttggcctagtttatgttcaaatagcaagtactcagaacagctgctgatgtttgaaattaacacaagaaaaagtaaaaaacctcattttaagatcttacttacctgtccataattagtccatgaggaataaacaccctttccaaatcctcagcataatgattaggtatgcaaaataaatcaaggtcataacctggttcatcatcactaatctgaaaaagaaatatagctgtttcaatgagagcattacaggatacaaacatttgattggattaagatgttaaaaaataaccttagtctatcagagaaatttaggtgtaagatgatattagtaactgttaactttgtaggtatgataatgaattatgtaagaaaacaacaggccgggcgggttggttcacacgtgtaatcccagcactttgggaggctgaggcaggcagactgcctgagctcaggagttcgagaccagcctgggcaacacggtgaaatcccgtctctactaaaaatacaaaaaaattagccgggtgtggtgacacatgcctgtagtcccagctacttgggaggctgaggcaggagaatcacttgaacctgggaggtgaaggttgcagtgagccaagatggcaccacttcactccagcctgggaaacagagcaagactctgtctctgagctgagatggcaccacttcactccagcctgggaaacagagcaagactctgtctcaaaaaaaacaaaacacacaaacaaaaaaacaggctgggcgcggtggctcacgcctgtaatcccagcactttgggaggccgaggcgggtggatcacctgaggtcaggagttccagaccagccttgtcaacatggtgaaacctccccccgccgtctctactaaaaatacaaaaattagccaggcgtggtggcaggagcctgtaatcccagctacttgggaggctgaggcaggagaatcgcttgtacccagaaggcagaggttgcactgagctgagatggcaccattgcactccagcctgggggacaagagcgagatttcgtctttaaaaaacaaaaacaaaacaaaaaaccatgtaactatatgtcttagtcatcttagtcaagaatgtagaagtaaagtgataagatatggaatttcctttaggtcacaaagagaaaaagaaaaattttaaagagctaagacaaacgcagcaaaatctttatatttaataatattctaaacatgggtgatgaacatacgggtattcattatactattctctccacttttgagtatgtttgaaaatttagtaaaacaagttttaacacactgtagtctaacaagataaaatatcacactgaacaggaaaaactggcatggtgtggtggctcacacttgtaatcccagtgctttgggaggctgagacaggagagttgcttgaggccaggagttcaagaccgacatggggaatgtagcaagaccccgtccctacaaaaaactttgtaaaaatttgccaggtatggtggtgcatacctgtagtcccagctactcgggaggcggaggcagaaggaatcacttgagcccaggagtttgaggctgcagtgagctacgatcataccacagcactccagcgtggacaacagagtaagaccctatctcaaaaacaaaacaaaacaaaacaaacaaaaaaaaccacaagaaaaactgctggctgatgcagcggctcatgcctgtaatcccagtattttgggaggcccaggtgggcgtatcacctgaggtcaggagttagagaccagcctggccaacatggtgaaaccccatctctactaaaaatacaaaattagccaggcatgtggcacgcgcctgtagtcccagttactgggaggctgaagcaggaggatcacctgagcccgggaggtggaggttgcagtgagccgagatcacaccactgcactccagcctgggtgacacagcaataccctacctcaaaataaaaaagaaaaagaaaagaaaagttgctgtccccgctaccccaatcccaaatccaaacagcctctctcatctcacagtaagggggaaaaatcacccaaaaaagctaagtgatcttttgaaaacccaaactcttagaagtctaagattattatagtcaactcatgaagtgtcatcataaaagatactctaatattatttaagtagaaccacatattggttgtcttggtatgtctagcccctggcatacaaaatatttaataacactgatatggtacctgtgatgtgaaaatgtactatgagtacagctttataaatactatatatgtacctatatacagaaaaaaatacaacaaaatcataaaagcacttatctttgaaagaggagttacagcaattttatttagttctttattgctttgctatatattctaaatttttttcaatgaatatatatcacttttaaaaaaattcaatggtctttcttataaattatctttggcagcatgcgtttttatatatacatataaaatgtatgggaaatttttaaaggatacattaaattaaagcaaaatatacaaacaaaaaatcagaatacaaaaagataaaaagattgggaagggagggagggagtaaggaggaagggtgggtgggtatagagaaatataccaaataatggtaagaagtggggtcttgacactttctacactttttttaaataaaaaaaatttttttctctctcttttttttttttagagacgaagtctcgctatgttgcccaggctggtcttgaactcctgggatcaagagatcctcctgcctcagcctcccaaggtgcttggattacaggtgtgagccaccacgcctggtcactttctacactttaatatatatattttttcattttcaatgtcatttttattagttaatttataatacccattcaccattatattcaaagtctatttgaagaaataaaccagaaagaatgaaatactctagctcacatgctattcaatactaaattacctttcaaatcacattcaagaagctgatgatttaagctttggcggtttccaataaatattggtcaaaccataattaaatctcaatatatcagttagtacctattgagcatctccttttacaacctaagcattgtattaggtgcttaaatacaagcagcttgacttttaatacatttaaaaatacatatttaagacttaaaatcttatttatggaattcagttatattttgaggtttccagtgctgagaaatttgaggtttgtgctgtctttcagtccccaaagctcagttctgagttctcagactttggtggaacttcatgtattgtcaggttggcccgtaatacctgtgggacaacttcagcccctgtgcacatggccaggaggctggttgcaaacattttcaggtaggtggaccaggacatgcccctggtcatggccaggtggaggcatagtgctatacagcaggcagaagtcaatattgatttgtttttaaagaaacatgtactactttcataagcagaaaaaatttctattcttgggggaaaagattatgccagatcctctaggattaaatgctgatgcatctgctaaaccttcacatatcagaacatatttactatagaaagaatgaaaatgggacatttgtgtgtcacctatgtgaacattccaaaaatattttacaacaactaagtattttataaattttatgaactgaaatttagttcaagttctaggaaaatacaaaccttgctagatattataaaaatgatacaatatatattcatttcaggctcatcagaatatatctgttatcacttgacaagaatgaaaatgcaccattttgtagtgctttaaaatcaggaagatccagagtactaaaaatgacttcttccttgaagcttactcaccaacttcctcccagttactcactgcttctgccacaagcataaactaggacccagccagaactcccttgaaatatacacttgcaacgattactgcatctatcaaaatggttcagtgcctggctacaggttctgcagatcgactaagaatttgaaaagtcttgtttatttcaaaggaagcccatgtgaattctgcccagagttcatcccagatatgcagtctaagaatacagacagatcagcagagatgtattctaaaacaggaattctggcaatataacaaattgatttccaatcaaaacagatttacataccatacttatgtcaagaagttgttttgttttattgcatcctagattttatttttttgatttatggtttactttaagcataaaaaatttgtcaatacaactcttcccaaaaggcataaacaaaaattcataaaacttgcatcacttgagatacttcaggtatgaattcacaactttgttacaacttactatatatatgcacacatatatatatatttgggtatattgggggggttctaatttaagaaatgcataattggctatagacagacagttgtcagaacttggcaatgggtacgtgcaggttcattataccaagtctacttgtagttgttcaaaatgtatcataatacaaggccgggcgaggtcgtcacgcctgtaatcccagcattttgggaggctaaggcaggaggattgcttgaggtcaggagtttgtgaccagcctgggcaacagagcaagaccctgtctccaaaaagaaaaaaaataattttttacaaaataaaaacaaaatgtatcatcagacgaaattaaataagaggcaattcatttaaatgacaacttttcccagcttgacatttaacaaaaagtctaagtcctcttaattcatatttaatgatcaaatatcaaatactaattttttttttttttttttttttgagacggagtctcgctctgtcgcccaggctggagtgcagtggcgcgatcctggctcactgcaagctccgcctcccgggttcacgccattctcctgcctcagcctcccgagtagctgggattacagacatgcgccaccacgcccggctaattttgtatttttagtagagatggggtttctccatgttggtcaggctggtcttgaatttcccacctcaggtgatctgcctgcctcagcctcacaaagcagtagctgggactacaggcacccaccaccacacttggttaattcttttgtattttttttgtaaagacgggatttcaccatgttagccaggatggtctcgatctcctgatctcatgatccgcccgcctcagcctcccaaagtgctgggattacaggcgtgagccaccccgcccggccatcaaatactaattcttaaatggtaaggacccactattcagaacctgtatccttatcactaatatgcaaatatttattgaatacttactatgtcatgcatactagagagagttagataaatttgatacagctaccctcacagaacttacagtgtaatagatggcatgacatgtacatgagtaactgtgaacagtgttaaattgctatttaaaaaaaaagacggctgggcgctgtggctcatgcctgtaatcccagcactttgggaggccaaggcaagttgatcgctcgaggtcaagagttcgagaccagcctggccaacgtggtaaaaccccgtctctactaaaaatacaaaaaaaaaattagccaggcatggtggcacaggcctgtaatcccagctactagggaggctgagacatggagaactgcttgaatccaggaggcagaggttacagtgagccgagatcataccactacactccagcctgagtgacagagcgagactcctgtctaaaaaaaaaaaaaaaaaaaaagatacaggttaagtgttatggtagttgaagagagaactcaaactctgtctcagaagcctcacttgcatgtggaccactgatatgaaataatataaataggtataattcaataaataggaacttcagttttaatcatcccaaacaccaaaacttcctatcaaacaggtccaataaactcaatctctataagagctagacagaaatctacttggtggcctataatcttattagcccttacttgtcccatctgatattaattaaccccatctaatatggattagttaacaatccagtggctgctttgacaggaacagttggagagagttggggattgcaacatattcaattatacaaaaatgcattcagcatctaccttgattaaggcagtgtgcaacagaatttgcaggagagtaaaagaatgattataaatttacaacccttaaagagctatagctgggcgtggtggctcatgcctgtaaatcccagcactttgggaggctgaggcgggtggatcacctgaggccagaagttcaagaccagcctagccaacatggcgaaaccctgtctctacaaaaaatacaaaaattagccgggtgtggtggcacgtgcctgtagtcccagttacttgggaggccgaggcaggagaatcgcttgaacctaggaggtggaggctgcagtgagccgagattgtgccactgcactccacttcagcctgggcgacaagagcaagactccgtcacaaaaaaaaaaaaaaaaaaaaagcttaaaatctagtgggaaaggcatatatacatacaactaactgtatagcataataaagctcataatctgtaacaaaatctaattcgacaagcccagaaacttgtgatttaccaaaaacagttatatatacacaaaaagtaaacctagaacccaaagttacccagcaccaatgattctctccctaagcagtatcaagtttaaagcagtgattacattctactgcctagattgtaaactgagtaaaggagaccagcacctttctgctactgaactagcacagccgtgtaaaccaacaaggcaatggcagtgcccaactttctgtatgaatataagttacatctgttttattatttgtgacttggtgttgcatgtggttattatcaacaccttctgaaagaacaactacctgctcaggctgccataacaaaataccacagactgagtgacttaacagaaacttatttctcacagttttggaggctgggaagtccaaaattaaggtacctgcaaggtaggtttcaatctcaggcctcttctttggcttgaaggtcttctaactgtgtgctcacatgacctcttctaacaagctctctggtgtctctttttttttttttttcttttttgagacagagtctcactctgtcacccaggctggagtacagtggcacaatctgggctcactgcaacctccaactcccgggttcaagtgattctcatgcctcaccctcccgagtagcttggatgacaggagcccgctaccacacccagctaatttttgtatttttagtagagatggtgtttcactacattggccaggctggtctcaaactcctgacctcgtgatccacccaccttggcctcccaaagtgctgggattacaggtgtgagccactgcgcccgtcctggtgtcttttcatataagggcactaatccaatcagacctgggcccaaccctcccgacttcttctaactgtaattaccttccaaaggccctgtctccaaataccatcacactgggggttaggacttcaaaaaaggtatggggggggtgtgggaggacataaatgctcagtccataacaagcacccaacataaaaatggctagaacagatcacaaaaaaaaggtcctgtatggctttggggaagggctcaaccccaaaatatctgagagctctggaggggcctagaagtggtaaatgaatgaaaacgtggttactctccagatctgcctttcccaaatatggccattcttggctgaatcagaaatcaaaggacaggttattaattactagctctaagttacttaccatttgctgagacagttcagaaatctgactgcatctcctcagagatctagaacacagttctcaaattctaacttacttgtgatatacttgtgaatgataaaaatcgctacaggtacttttattaatctgaaagagtattgagaaattacctttcattctgacttttgtctggaatgaaaatcaatacttttgctataatcgattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttaaaaaggaaaaaaaaaaaaaagacatgttggccagatgcggtggctcacacctgtaatcccagcactttgggaggctgaggtgggcggatcacccctggcctgcccaacatggtgaaaccccgtctctactaaaaacacaaaaattagctgggcgtggtggcgggcgcctgtaattccagctactcgggaggctgaggcaggagaattgctagaacccaggaggcagagattgcagtgagctgagattgcaccattgcactacagtctgggcaacaaaagtgaaactccatcttaaaaaaaaaaagacaatgttcgtgggtccaaacaagacttaatggaagtgagtctaaaaatgagctatgtgggccaggcgtagtggctcccacctgtaatcccagcactttgggaggccgaagcaggcagatcatgaggtcaggagatggagaccatcctggccaacacggtgaaatcctgtctctacaaaaattagctgggcgtggtggtgcctgcctgtaatcccagctactcagaaggctcaggcaggagaatcgcttgaaccagggagtcggtggctagagtgagccgagatttgcatcactgcactcctgcctggtgacagagcaagactccatctcaaaaaaaacaaacaaaaataaaagataaaaatgagctatgtgaattaaaagaggtataacaatagataaaccatattttatttaattcctagtaatgagtaatatttccaaacttctggaatgggcagaaattgctagttggcatatttttaccttttatattcagatacattaaaattctcaaaaaaaaacacctcaaagcagatgatccgccatctccttggataatttgtgttaactcaggataacagaaaaccaaaattatgagttactgatgcaatattcctaaatgtaaaaataattaaagctaatagtagattcatcttccaatttcatatcagtcttacaaataaactacatatataacttgcttgccttcccttctgagggataaagctgttagaagaattaaaatcagcattcttgactattcaaccaagggagggataaattattactcattctagggacatgggctcataactactacatgtgtaaggacatgaatttacccaatattacaatttttccttttattagtgtgtacagtggaagaatagacatgttcactctggacaaaaaaaaaattatacttatcagttatcagaagcacaatgctgaagacagtagttccataacaatttgaagtatgtgatcgaactagtagattatcttagtagtagtgaattattgtaaatgttagtaatttggcagccactgggcagaaaaataagaattgaggctcaatattgatattaatggtggtgattgacacataaattttatcaagtctacacaatataaaattacagaaaggtagaagagtataccagtacaacttcaacatatcttcactacaagggagtaaaatgacatggcctagttactatctaatgaactgcagaaaactaaaagaaaactccaaggcaactcttctctgctgatctggttggtccttttcctaccttttgcaatacccagatacaaacaatggatagaaaacaaagtagacttgtagtatgcaggtcacagtgctaaattcacagaaagaaacccctgaactgaactgctctatttcctggtggtcacaaagagtaattctggtttacacctacagattgatgtcaatctacaccctgttgataacagtgtggccaaggacaaaaaaaaggtgctccgttttaccaattctgtaaaaaattattggcagggtaagctcggctagggcaggattacatttctaggactaccatccccgaaatttagaagatattatatccacataaagcatatctttcacattaatttgcaaaaatctaaaagctttttcttagctcaagtgtgtccaagtttaccctggcagtttaaaacgatagttacaagcagcatgggttgtatcagacacatttgagggccaatttcatgtaagtgatattgggcaagttacttcaactatctgtgcctccaaggtcatactagtgtttatttacctaaagggtacctgttatgtaactttagggtgtttacattagataatgcctgcaaaatatttacttcaacgcctaaaacatagttaagtattcaataaatacctactattgtcactactaacttaaaagtttagagattaagagcagaatctggggtgagacaaacttaggttcaaatcctagtattgttgggtaatcttgggcaagttacttaacctctctgatttgtgtaatttaaaaaattagttaatatacataacagggcttagaagagtatctagcacatagcaccatttaagcatttgttattgctaacatgcaaacaatttaagggaaagaaattttttaaaaaggaagagggatttgcaaactaaaaacaatgagtatcttatgttcaaagaaaactaacaaacagccagctctagcaataattaaattcactatatactggggcaggcatcacaccccaaagctaaaagcgtctacctaggccaggcacggtggctcatgcctgtaatcccagcactttgggaagcagaggcgggcagatcgcttgagctcaggagttcaagaccagcctggacaacatggcaaaacaccatctctacaaaaaatacaaatattaggccgggcgcagtggctcacgcctgtaatcccagcactttgggaggccaaggcgggtggatcacctgagatcaggagttcgagagtagcctggccaacatggtgaaacctcgtctctattaaaaatacaaaaaattagccaggcatggtggcaggcgcctgtaatcccagctactcagggggatgaggtaggagaatcgcttgaacccgggaggcagaggttgcactgagccgagatcatgccactgtactccagcccgggcaacaagagcgaaactccatctcaaaaaataaataaataaataaataaaataaagtacaaatattagccagggatggtggtgcgcacctgtagtcccagctacttgggaggctgaagtgggagaatcccctgagcctggggagaatcacccgagcccgggaagtcgaggctgcagtgagcagtgattgtgccactgcactccatcctaggtgacagagtgagaccctgtctcaaaaaaaagaaattggcagaattaagtaagttgatgtttagagatgaaaaatcaacattttttcctcagcaactgaataaaaacaacagccactaccatttttttgagtacctatttgtagcctattttttaactggtattactcgagagagagagagctaggttcgagacagagctccttctcttaataactgtatgacctagggtatgtctgttagcctctctgaggcttcaaaggttcctcatctgtaaaatggtaataatcataccattgctacagggctgttttgaagactaattaggactatgtaagtaaacatgatgatggctattattactgttccccgccaggggccatgcaagggttgctgattcacatagactgtcttataatcctctcaataactccaagaggtagccagcacctcagatatacataaaatgacttaagcccagagaggtgaagtaagttgcccacagccacacaactagtaaatagcccaaacaagctggattcccagttagactccgttaatagcactgctctttaccttaagtcattacaatgcctaatatgaaatagaatcgcttctttcttagggttcaagtggttaattatttaatgtattcattcaacaaaccatcatcgaggacctcttacaagccaagtactgtgctaagtgctagagttacggcggtgattcctgcccttaaaaagttttagtgggagaaacaacaggtaaccaggtcattgccaaaacaacaaaaataatcataataaagcaggctaaagcatatttaactggccggggttttgactattttagcaagcatgatcagaacggttgaggagggaggccagcagcttggccggttcaacaaacaagaaaaaaccagtgagggtggagctaagataccagaggctgattacggttaagaatgttcttgaaggtaaggaccagattctcattttctatatcctggggcatcggtcagcatggaatctggattctagcacatgtgaatttcggcttgaaatgacctaatgccttttccctagttccttcgtgtgtcaaatacgcatggttaccgctaccagagctgtagtggggcttcaatgaggccatgagcatctccataaagatgaactacagtgtgtgcaaaactaaaggcaaaacctggtccccacacgccctcccaggtggtcgctttccgtgccgaggcccctccagaggtgccccgagaacctcaccatcgcaccccaaacttccagggaagggcctctcccgagaaagcccccacgcccccaccccgcgccatcattcccgaatctgccctcggcccctccccgcagcacgctcgcaggcggcacatgtcaaccaaaacgccatttccaccttctcttcccacacgcagtcctcttttcccagggctcccccgaggagggacccaccccaaaccccgccattccgtcctccctgccgccctcgcgtgacgtaaagccgaacccgggaaactggccgcccccgcctgcggggttccctgggcccggccgctctagaactagtggatcccaattgaaggcctggtctaaatgactccaaaatcaccacttaattcaagagactgatttccctgagtcaggccccttaaagcagctatttcaatgggacagggaaacaaccctaggatctggattagaatcacttgggggctgccacacccccagggctctgatcctgcccttctcccacacgcacattcacatactgctgcagtgaccttccatttctaatgggttcctgggccatctgtcaggtatagggaatggaaaaggggttggggaggctctgcttcagaaagtttgtgtcaggggctcccagagcctccacagatagatagcaggggtccccaccctaccatggcagctataaatgtgatcaacatttattggcctaggatacagcagttagcaaaatgcctgatgtagttcccactccgtggaggttgcaggctagccaagaagtcatgagttcagcaacccttacgcaccagtgggatgagattggaccaggccgagggtagtcttgggaacactcagcatttgtctgagggccagaagaggctgcttgccctcagacaggaggtcagcatctttattgtagcccatgacacctctacaccattgctcttctggtcttatggaagacatctttgggcctgataacagcggagtctgtgtcccacttgtccaggctggagtgccacatcaggcacactccagttgcagggacagcacagacaagtttcaggaaggctggtggcctccaggaggttaaccttataaggccagattgtaacctagttgaaaaacatacacatgccatgataataaaagaacctaggcaccattacaagagaaaaaatcatttttgtagatacgagcatggattcttgggtgggtcagacacactgggcttgtgctctgactgcactgtctcccctacctgaccttgggtaaaccataagactgctgcatgactcagtgtccaccccaaaaaagtaccggtagatattggccacagtagatatcagctagagtggactctcatgacaatgaggggagatgtattccccatcttaggcacctgggactctaccttccatcttctgctccgtgtctctccatccccaggctcttcagaactcagggagtccagaatgtcagctcccagatttcagccttcagaaaggaaacccattaccgttcagttgaacaaatgttgtctgagccccagatctgggctcagaggccatctaggctatgagacaagaggggaacaaagcaccgtctgcactcactcaccacactcacttgctgtcccaggtcacatccatcgggtagagaatctaagaggctgagctagctcccgccaccagcccagcccaccccacctggccccttccttccttctacaaaatatgcaccacctgtcaaagggtgggcagtgccaggcctgcatacagagcactgagtgtaaaagcagacatggaccctgacctccaggagcttccaattttcttgaagagacaaatcagctggcatttcagtccagtgtgatctgctcttggtgagcacagacctagggagttggggcagcttcccagaagaactgcagtccaggctgagggcagagaaatgaggggaatggcgaggaattggggagcaggggggagctcagtagagagccaagggcgggaggtgagaagtccgtgttgggccaggagctaccctccggtggccacagccgaagtcgaggatgcctttggaactcatccccacttctctctttctgtatgtagccgtccaagaacaagtcacctccaagtgtagccggatcaaggcaagccccccatctagcaagcacttgatgccacccagaactgggcttcttcagaacaatctgagtccaggaatgatcccactcaccaggcaccagagctgcgagggcatgggagtgatctcaccaactctggggaagcggcaaggaattttcacctccagcccccagtgtcccatcctctcacactcaggccagactcccctgggcagacttgactctgtctgccagcatatgcagagccccaaggccaccccaccagaagtgcccctgcctgggttctgtcccagctccctgggcacccagtccttgagtccccaccagctcagacggcctagtgtgccaagaatgcccactgcgttcaacaatgctgcatgggtcacagcggcagcagctgtgaccacagcagtttcggggaaaacacccctcagccaagtggataatagcgttcagcagcactcaccttctggccaggcctgccttcagaggccatctgattgggaggcacaagtgcccgctgcgatgggaacacaagtgcccctggccaacaaccccagcttcagcctgctgggcagccagagcctcaggcagagcccggtacagggcccggtgcctgtagcaaacaccaccaagttcctccagcagggtatggccagctttagtcccctgagccccatacagggcatcgagccaccaagctatgtggctgctgctgccaccgctgctgctgcttctgccgttgctgccagccagttcccaggtccgttcgacagaacggatattccccctgagctgccacctgccgactttttgcgccagccccaacccccactaaatgatctgatttcgtcacctgactgcaatgaggtagatttcattgaagctctcttgaaaggctcctgtgtgagcccagatgaagactgggtgtgcaacttgaggctgatcgacgacattttggaacagcatgctgctgctcaaaatgccacagcccagaattctgggcaagtcacccaggatgctggggcactttaaatctgagcaggatgcccatagaaacccccatggtgacatcactctaggaagtggtgtcgatccatacccgcagttgtctcccgttacaatttgagtggtgttgtcagcccatgcttatccctctctctacctgtgacaaaatggaaagctggtgatttttcaagctacgtgtacatatttgaaaattttgtaaatggttttcctaaacattaatgacagaagtatttatacttcattttgtgactttgtaaataaagcgacggcttttgtttcagtagagttgtgtttactatgcattgttttgtgtttattatacaatgttacaaatatgcagaccgtgttgtttgctccagtgataccttgttaagctaggtggctgagtcgcttatggttttaatgcaatgagcaatgtggatatgaccaagagttgttgtgcaagttgacaaatgccaaatagaaaaccacttggccatttatttctatgttcactaaaaatcctattgccttgtgtgattcttaatctcttttgcgaacctttcagtctccgctagctctttcctaatgagctttacagcagaagctgttttatcgttaagtgccccacagagacactttaccaggaggctgggagagttctccagatttgggagaggcgcagagacagtgtgtgagccgagccctgtctcagcaatccacctggaggagctagagtatcctcctccctttaccattcagaccgagagaaaaagcccagcttgtgtgcaccctcgtggggttaaggcgagctgttcctggtttaaagcctttcagtatttgttttgatgtaaggctctgtggtttgggggggaacatctgtaaacattattagttgatttggggtttgtctttgatggtttctatctgcaattatcgtcatgtatatttaagtgtctgttatagaaaacccacacccactgtcctgtaaacttttctcagtgtccagactttctgtaatcacattttaattgccacctcgtatttcacctctacatttgaaatctggcgtctgtttcaagccagtgtgttttttcttcgttctgtaataaacagccaggagaaaagtgcctctatgtttttatttttcaagggagtattcagtacctacaaacccaagtcaggaagcctgctagtggctttggttctttcagaggctgctcgatgccttgtgtgtcagaaagaaagattcagcagttttgcatcatggcaaagaagcctgttattttggggctcagcccctcattttatagaggatgaaacagagggggatgggaggtcacaaagacaactgccccgggagcaggtgtgggggagacttgccctgagggtctagacgctctgcaccaccgtcctgtctcccttgctgaagaccacacatgcccttctttgaccagaccctgccacctgataggccaggacctggtaggcgggtacccaggtttcatggatggaaccacatctccccaaaagtggggaggtagctactgggatgcacgcctcccgccatgtgctataggagagcagctgaagcaacagttgggatcagatgtagtcacaattgaatgcatcatcacatttatccctctaagtggctgggagagttgatatcctcatccctaaggtacaaaatgttccaatttgatcagtggctttcaggagctgagaaaggcatgtgctctgaggcagagctgttatgtcccgcagagcctaaaaatgctctaagaacatgctccctgccaaaattctcaatggctgtgacaagggacaacgatcgaccaatgggggtggaagcagacctccgcagtccaggggccagagctaggacagaggggtcggagaaagagtcattttcccaacactccagctcttggccagtcctcacacagtcccctcctgcttcctgctgagagagatatcctcataggtctgggtaaagtccttcagtcagctttcattccctgtcaccaactttgtctctgttctccctgcccgtctcaggcagcactcctcaggaaacctctccaagagccagcctcactgcagcgcccactattgtccctctgcctcaagtgtcccatccatgccaggccccaggcaggctgcagctttccctcagggccacaccaaagcacttgggctcagctgtgctgtccccctccatcactgagctcaggggcagcaggggtggggtgccaggaggcccattcacccttctctggctctgtgttggacccacctgcccagccactgctgcttagaacctacccgctgggaaaatgaagccctcccggaggggccacctcaacctgagagcctcacggatcacagttgtccccactcagctctgccagccctcagagacccatagataaaagctgagcttggctcgcagagctggttccatcttccattcccagagggttcaacttcctaccccaaccacacagggaacctcaaggctgagccagtgtgggctgcagtgcagaccagcttcctggacacgtcctgccacctgaccccaggctggcctcactgcccctggcactcctgaccctatcctcattcctcctggcagtgcgtgttctgccattccgctttcccttagctgtcctctcactgtactgtcagcttctccttttccaggtgccccccaggggctttccacatgaccctgtcaccccacagcccatccagcaccaattccagctctctgccacccttcaaaggagtgacagtgccctgcttcacctcccactcacccctcaacccagagcaatctggctccagtcttgcctccttccccctaagtactctagtcacagttccaaattcctcctggtcataaagccaaatgaagcttcctggtcctcagcggacttgccacttcagcagtactggactctctcctcccagaaacctgtttccccttggctcctggagcccacactctgctggaatccttctgcctctctggcctgtagcctggccctctctcccaacctgaggtccattctctcctgctcctccacaagatgttgctccttccattacttcctccctctcaaccaaagctccttcattagctctttatcttctggtttcttcccctgggcagacgaatggattcaagagcctgtggcccagcagcccagcactccaggatctcagcacttcagcatcccagtaccctagcatctcaataccccagcaccccagcaccatagtattccagcaccccattgtccaagcatctcagcactccagcatcccagcaccccaacactccagcagcccagaatctcagcaccctagcactgcagcatctcaggaccccagcacttcagcatcccagcacactagtactccagcatctcggcaccccagcacctaggcatcccaacacccagcaccccagcacttaagcatcccaccactacagtatctcaacactccagcaccccagcaccatagtgttccagcaccccagcatcccaacaccccagcacttaagcatcccaacacctcggcatcccaacaccccagcactgcagcatctcagcaccttagcatcccagtgccctagcatctcaatgctccagcacaccagtactacagtattccagcaccccagcactccagcatctcagcactgcagcactgcagcactccagcatcccaaaatcccagcatcccaacaccccagcagaccagcagaccagcatctcagcaccgcagcatccaaggactatcccagcatcccagcaacccagcacctcagcatcccaacaccccagcatttcagcatggcaacaccccagtaccccagcacttcagcaccccagtatcccagcatctcagcgacccagtatcacaaaacctcagcatcctagcaccccagcaccccagcaccttagcaccttagcatcccagcatctcagcgcctcagcatcttgatattctggctgaggtcagcgtggtgtatctagtcagggtcctaactttcacttcgcagggaaatgctgctggactgggtctcatgttgggctgaagctctctagaccccttgaagacagcataaaagagcttggagacgctgggtgtcccccatggaagagttcactctcatcctgctttgacaacagccttctctggggtccctcacgggcccctctttcttactgcaagtttgtctctgagaagactgtgatgcagaagtcactcagctgcctgtggctcctgaagagctgaaggtggaggcctgtaggcctccctatgagaggcgcagaaaaaaccatgattgctagtggggaggtgctccctctacaacccactccataatctgcccccgcccagctctgaggccagccccaggggaaaatgccagatccccagggaggtgtgtgagacctcaggggctccctcctcccttacagcaggctcaggcccctgggggcctcagggccaaggtctgtgggtaagctactatctctcacttgtcctctagccacaaaagccagggagatctggcaatggacatgaggttctgaagaagcacatatgactggcttcctaatgcgtggttgttcagtgattcaataaacacgcatgggccaggcatggggaaatagacaaacatgatccccaacctctcccagagtgaactgggagggaggagtgttcatccctcaggattacaccagagaaacaaaccagcaggagatatatatggttttggggggtcaagaaagaggaaaaacctggcaaggcaagtccaaaatcataggacaggctgtcaggaagggcagcctggaacctctcaagcaggagctgatgctgcagtccacaggcagaatttcttcttcctcggggaaatctcagctttgttcttaaggcctttcaactgattggctgaggtctgccccttcccccacattctccaggataatcttccttacttaaagtcaactattaatcacagctacaaaatcccttcacagctacacatagatcagtgtttgattgacgaacagcccctacagcctagccaagttgacacataaaactaaccatcacagggggacaaatgatgtaaacacatcaacaaataaaacagtaacaagttaaggtctatggaaaaaacacagaaggggcagagagaaagaaagcaagaaggagagtcccagtttgctagggcttgtgggaagtggggagcagttctctttagctaggatatttgggaaaggcatatctgaaggagtgatatttgagcttagattaaaagatgggaaggagcaagccatgcaaagagctaggatgttccaagcagagacggaacagcaagtgcaaatgtcaggaggaatagaaggaggctggtgggtggggtccagtgagcaagaggagggcaggcaggagaggggatggggaggtgggcaggcccagaccacccagggccctggagactatcctgatccaacaagggaagccttgagtcacttcagtgtccatgtggagaatggacctcagactgaatgagggaggcagtaaggagggcctctacctccagggcttcgccctgtggactgcgcatagacatctccaactcagaaagtctgaaccaaactttccatagttcccccaagtctgggcatcctcctactcagtgaaaggcagccatcacacctccctgccctgctcccggatgccccaaatcctcttggtctccaagtccagaacctgagacttgtccttgatgtttgtctttccctcaccctttctgtattctgggaagatgggtttttttcccccagatgaatctgtaaaacttctgtgatcacaataaaaattctggcagtattattttctggaacatgacaaagtgattcaaaattatttatctggaagactacaaaacaagaatagccaggaaatttctaaaaagaaagaagaaggaggaggagaaagaaggaggaggaaaaggaggagaagaagaaaagaaaaagaaccaagaaagggttctagctctaccaaatattaaaacatatcatgaagctatttaaaacaatatggttgtggatactgaaaaagatgtgaataaagtggaaggaaaataaatagaaatgcacatggggattgagactgtgaaaaaggcagcatctcacatcagtgagggatgttcaacacctggtgttgggaaaactggctagtcatttaaaccaaacaactgggtcctctacctcactcctgacattaagatacatttagatgattcaaagagtaagacagaaaaaataacacgtgaaaacactatcagaaaacaacgtgggccaggtgtggtgggtcacgcctgtaatcccagcactttgggaggccgaggcagacagatcacctgaggtggggagttcaagaccagcctgaccaacatggtgaaatcctgtctctactaaaaatacaaaattagctgagcgtggtggcgcatgcctgtaatcccagctactcaggaggccgaggcaggagaatcacttgaacctgggaggcagaggttgtggtgagccgagatcacgccattgcactccagcctgggcaacaagagtgaaaatccatctaaaaaaaaaaaaaaaagccaaggtggatatttttatagtatcagggtagatcaagcttctccaatcatgacatgaaacccagaaaccataaaagaaaagaatgataaaattgcccacgtaaagtaaaaagcttgcacacagaaaaacaccatacaggttacaagatgagcagcaaaatcagagaaaaaacattgcaattcaggacacacagaggctattgttcctaatatttaaaaataaaagtagtggattgtctacaaaaagatgaagacaagaatttcagaaaaccaaatactgcatgttttcacttacaagtggaagctaaacactgagtacacgtgtacacaaagaatggaaccataggccaggcaccgtggctcacgcctgtaatcccagtactttgcgaggccgaagcgggcggatcacctgaggtgaggagttcgagaccatcctggccaacatggtgaaacccagtctctactaaaaatacaaaaattagccgggcgtggtggtgggtgcctgtaatcccagctactcgggaggctgcggcagtagaatcgcttgaaccctggaggtggaccttgcagtgagccgagatcgcaccactgcactccagcctgggcaacagagtgagactccatctcaaaaaaaaaaaaaaggaatagaacaatagacactggggcctacttgagggaggagggtgaggatcaaaaacctgcctatcaggtactatgcttattacctgggtggtgaaataatctgtacaccaaaccccagtgacatgcaatttaccgatgtaacaaacctgcccatgtacccgctgaacctaaaataaaagttggaaaaaaatatagaaattttctttgtaatagccaaaaactgcaaacagcccaggtgtctattagtagaatgcataaacaaactcgggcatgttcatacaatgtaaaactactcatcaataaaaagtgatacttctcagcaatgaaaagaaactagctactgataccagctacaacatggatggatttcaagtgctttatgatgagagcaagaagccagacacaaaagtgtctatatatatatacagtatatatacgtatatatacacatatatacagtatatatatacatatacatgtatatatatactgtatatatactgtatatatatacacagtatatatatacatatatacagtgtatatatactgtgtatatatacatgtatatatactgtgtatatatacatgtatatatactgtgtatatatacatgtatatatactgtgtatatatacatgtatatatatgtatactgtatatatactgtatatatatatacacatatatacagtatatatatacagtatatactgtatatatacagtatatacgtgtatatatacatatatacagtatatatgtaaatatacatatatacagtatatatgtaaatatacatatatacatgtatatatatacactatatatatacatatatagtgtatatatacatatatacatgtatatatttactatatgattccatttatataaagtgccaaaacagtcaaaaataatctatgtggaaaaaatcaacaaagggatcccccgggctgcaggaattcgatggcgcgccttaattaaaattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtaccagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaaggctagcctgcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcatctgccttctccctcctgtgagtttggtaagtcactgactgtctatgcctgggaaagggtgggcaggaggtggggcagtgcaggaaaagtggcactgtgaaccctgcagccctagacaattgtactaaccttcttctctttcctctcctgacaggttggtgtacagtagtagcaagcttaaggatctagactgccatgggcgtgcacgagtgccccgcctggctgtggctgctgctgtccctgctgtctctgcccctgggcctgcctgtgctgggagcccctccccggctgatctgcgacagccgggtgctggaaagatacctgctggaagccaaagaggccgagaacatcaccaccggctgcgccgagcactgcagcctgaacgagaatatcaccgtgcccgacaccaaggtgaacttctacgcctggaagcggatggaagtgggccagcaggccgtggaagtgtggcagggcctggccctgctgtccgaggccgtgctgagagggcaggccctgctggtgaacagcagccagccctgggagcctctgcagctgcacgtggacaaggccgtgagcggcctgcggagcctgaccaccctgctgagggccctgggcgcccagaaagaggccatcagcccccctgatgccgcctctgccgcccctctgcggaccatcaccgccgacaccttccggaagctgttccgggtgtacagcaacttcctgcggggcaagctgaagctgtacaccggcgaggcctgccggaccggcgatcgctgaggatccccatccagcttggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattcagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcaagacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcttaattaaggcgcgcccctaggggccggccttaattaaatcaagcttatcgataccgtcgaacctcgagggggggcatcactccgccctaaaacctacgtcacccgccccgttcccacgccccgcgccacgtcacaaactccaccccctcattatcatattggcttcaatccaaaataaggtatattattgatgatgtttaaactacggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgcgacgcggacgcgcgtaatacgactcactatagggcgaattggagctccactacgtagtttaaa

SEQ ID NO: 19 - DNA, 582 bp, Homo sapiens
  misc_feature: Human EPO
atgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagatga

SEQ ID NO: 20 - DNA, 582 bp, Artificial sequence
  Optimized sequence of human EPO
atgggcgtgcacgagtgccccgcctggctgtggctgctgctgtccctgctgtctctgcccctgggcctgcctgtgctgggagcccctccccggctgatctgcgacagccgggtgctggaaagatacctgctggaagccaaagaggccgagaacatcaccaccggctgcgccgagcactgcagcctgaacgagaatatcaccgtgcccgacaccaaggtgaacttctacgcctggaagcggatggaagtgggccagcaggccgtggaagtgtggcagggcctggccctgctgtccgaggccgtgctgagagggcaggccctgctggtgaacagcagccagccctgggagcctctgcagctgcacgtggacaaggccgtgagcggcctgcggagcctgaccaccctgctgagggccctgggcgcccagaaagaggccatcagcccccctgatgccgcctctgccgcccctctgcggaccatcaccgccgacaccttccggaagctgttccgggtgtacagcaacttcctgcggggcaagctgaagctgtacaccggcgaggcctgccggaccggcgatcgctga